(19) United States
(12) Patent Application Publication
(10) Pub. No.: US 2026/0049318 A1
(43) Pub. Date: Feb. 19, 2026

(54) INHIBITION OF PLANT PROTEASE AED3-1 FOR IMPROVED HETEROLOGOUS PROTEIN PRODUCTION

(71) Applicants: University of Guelph, Guelph (CA); PlantForm Corporation, Toronto (CA)

(72) Inventors: Jennifer Geddes-McAlister, Guelph (CA); Nicholas Prudhomme, Guelph (CA); Doug Cossar, Toronto (CA); Michael D. McLean, Toronto (CA)

(21) Appl. No.: 19/298,731

(22) Filed: Aug. 13, 2025

Related U.S. Application Data
(60) Provisional application No. 63/682,542, filed on Aug. 13, 2024.

(51) Int. Cl. C12N 15/113; C12N 15/82

(52) U.S. Cl. CPC C12N 15/1137; C12N 15/8205; C12N 15/8242; C12N 2310/531

(57) ABSTRACT

The disclosure relates to methods for enhancing production of a heterologous protein in a plant or plant cell by inhibiting aspartic protease AED3-1. Also provided are plants and plant cells with enhanced production of a heterologous protein, wherein the plant or plant cell has reduced or loss of function of AED3-1. Further provided are shRNAs and vectors for use in enhancing production of a heterologous protein in a plant or plant cell.

### RELATED APPLICATIONS

This application claims the benefit of U.S. Provisional Application 63/682,542 filed on Aug. 13, 2024, herein incorporated by reference in its entirety.

### INCORPORATION OF SEQUENCE LISTING

A computer readable form of the Sequence Listing “P96529387US00_96529387_SequenceListing” (9.9 kilobytes), submitted via Patent Center and created on Aug. 13, 2025, is herein incorporated by reference.

### FIELD

The present disclosure relates to compositions and methods for increasing the expression of heterologous proteins in plants by inhibiting aspartic protease AED3-1, as well as plants with increased expression of heterologous proteins.

### BACKGROUND

As the population ages and medical conditions and diseases become more prevalent across the globe, the demand for therapeutics is rising. For instance, biologic drugs, including insulin, growth hormones and antibodies are among the fastest growing categories of therapeutics under development today (Haydon, 2017). While most biologics are generated via mammalian cell systems (Kantardjieff and Zhou, 2013; Zhu et al., 2017), alternative approaches, including biologic production via microbial-based systems (Lee and Jeong, 2015; Wang et al., 2021) or with the use of insects (Yee et al., 2018), are also performed.

Another alternative approach is the use of plant-based production systems, referred to as molecular pharming, which provide a cost-effective, lifestyle-conscious approach to making new and similar medicines to combat disease (Buyal et al., 2021; Shanmugaraj et al.,2020; Stoger et al., 2014). Additional benefits include reduced industrial costs, manufacturing scalability, assembly of complex proteins, addition of eukaryotic post-transcriptional modifications (PTMs), lack of animal components and swift deployment of cutting-edge therapeutics (Buyel et al., 2017; Chen and Davis, 2016; Fischer and Buyel, 2020; Schillberg and Finnern, 2021; Shinmyo and Kato, 2010). Recently, the efficacy and safety of a recombinant plant-based adjuvanted COVID-19 vaccine was demonstrated through phase 3 clinical trials (Hager et al., 2022), emphasizing the importance of plant-based approaches for supplying therapeutics for the prevention and treatment of sustained and emerging diseases.

The process of molecular pharming capitalizes on the host-pathogen interactions between the common plant pathogen (Agrobacterium tumefaciens; updated nomenclature to Rhizobium radiobacter) and the plant host (Nicotiana benthamiana) to deliver genes encoding for the desired protein product into the plant cells. These products are often therapeutic proteins, including antibodies, vaccines and enzymes with applications across multiple sectors (e.g. medical, veterinary, agriculture; Shillberg and Finnern, 2021; Tschofen et al., 2016). Despite exploitation of a natural infection process and advantages associated with plant-based systems, widespread use of plant-based pharming technology is still limited. Specifically, constrained expression and accumulation of recombinant proteins due to the plant's natural defence responses against pathogenic invasion, including transcriptional activation of defence genes prior to T-DNA transfer (Veena et al., 2003; Xu and Pan, 2000), increased H₂O₂ production, hypersensitive response (Lee et al., 2009) and unintended proteolysis (Grosse-Holz et al., 2018b) that reduce biological output. To capitalize on the molecular pharming production pipeline, further innovations are required to minimize the deleterious effects of these defences and to support production and accumulation of the target protein.

### SUMMARY

The inventors have shown that inhibiting aspartic protease AED3-1 enhances the production of a heterologous protein in a plant or a plant cell.

Accordingly, in an aspect, herein provided is a method of enhancing production of a heterologous protein in a plant or plant cell comprising:
a) introducing into the plant or plant cell a nucleic acid molecule encoding the heterologous protein operably linked to a promoter;
b) inhibiting aspartic protease AED3-1; and
c) growing the plant or plant cell to obtain a plant that expresses the heterologous protein.

In an embodiment, step a) further comprises introducing into the plant or plant cell a nucleic acid molecule encoding a suppressor of gene silencing protein operably linked to a promoter, optionally P19.

In an embodiment, the plant or plant cell is of genus Nicotiana, optionally Nicotiana benthamiana (N. benthamiana).

In an embodiment, the AED3-1 comprises i) an amino acid sequence according to SEQ ID NO: 1 or ii) an amino acid sequence with at least 60% sequence identity to SEQ ID NO: 1.

In an embodiment, the inhibiting in b) comprises contacting the plant or plant cell with an inhibitor of AED3-1.

In an embodiment, the inhibitor comprises a polypeptide and/or a small molecule drug.

In an embodiment, the inhibitor comprises a short hairpin RNA (shRNA), a small interfering RNA (siRNA), a micro RNA (miRNA), or an antisense oligonucleotide (ASO).

In an embodiment, the inhibitor is the shRNA.

In an embodiment, the inhibitor comprises a nucleic acid sequence according to SEQ ID NO: 2 or a functional variant thereof.

In an embodiment, the inhibiting in b) comprises introducing into the plant or plant cell a nucleic acid molecule encoding the inhibitor of AED3-1 operably linked to a promoter.

In an embodiment, the inhibiting in b) comprises introducing into the plant or plant cell at least one genomic modification in a gene encoding the AED3-1, wherein the at least one genomic modification confers reduced or loss of function of the AED3-1 as compared to a plant of the same species lacking the at least one genomic modification.

In an embodiment, the at least one genomic modification conferring reduced or loss of function comprises an insertion, a deletion, and/or a substitution.

In an embodiment, wherein the method comprises targeted genome modification.

In an embodiment, the inhibiting in b) further comprises introducing a guide RNA (gRNA) into the plant or plant cell, wherein the gRNA is complimentary to a nucleic acid target sequence in the gene encoding AED3-1.

In an embodiment, the gRNA is a single guide RNA (sgRNA).

In an embodiment, the inhibiting in b) further comprises introducing into the plant or the plant cell a nucleic acid encoding an endonuclease operably linked to a promoter.

In an embodiment, the endonuclease is a Cas9 or a Cas12 endonuclease.

In an embodiment, wherein the introducing into the plant or plant cell the at

least one genomic modification comprises chemically-induced mutagenesis.

In an embodiment, wherein the heterologous protein is a biologic drug. In an embodiment, the heterologous protein is an antibody. In an embodiment, the antibody is a human antibody or a humanized antibody. In an embodiment, the antibody is trastuzumab. In an embodiment, the heterologous protein is insulin. In an embodiment, the heterologous protein is a growth hormone. In an embodiment, the heterologous protein is a vaccine. In an embodiment, the heterologous protein is a therapeutic or diagnostic protein.

In an embodiment, the introducing into the plant comprises agroinfiltration, optionally with Agrobacterium tumefaciens (A. tumefaciens).

In an embodiment, the A. tumefaciens is grown in a bioreactor prior to

agroinfiltration. In an embodiment, the heterologous protein is harvested less than about 7 days post-infiltration (dpi) or at about 3 dpi.

In an aspect, herein provided is a modified plant or plant cell with enhanced production of a heterologous protein, comprising:
a) a nucleic acid molecule encoding the heterologous protein operably linked to a promoter; and
b) at least one of the group consisting of:
   i) an inhibitor of aspartic protease AED3-1; and
   ii) at least one genomic modification in a gene encoding aspartic protease AED3-1 conferring reduced or loss of function of the AED3-1 as compared to a plant of the same species lacking the at least one genomic modification.

In an embodiment, the modified plant or plant cell further comprises a nucleic acid molecule encoding a suppressor of gene silencing protein operably linked to a promoter, optionally P19.

In an embodiment, the plant or plant cell is of genus Nicotiana, optionally Nicotiana benthamiana (N. benthamiana).

In an embodiment, the AED3-1 comprises i) an amino acid sequence according to SEQ ID NO: 1 or ii) an amino acid sequence with at least 60% sequence identity to SEQ ID NO: 1.

In an embodiment, the inhibitor of AED3-1 comprises a polypeptide and/or a

small molecule drug.

In an embodiment, the inhibitor of aspartic protease AED3-1 comprises a short hairpin RNA (shRNA), a small interfering RNA (siRNA), a micro RNA (miRNA), or an antisense oligonucleotide (ASO).

In an embodiment, the inhibitor of aspartic protease AED3-1 is the shRNA.

In an embodiment, the shRNA comprises a nucleic acid sequence according to SEQ ID NO: 2 or a functional variant thereof.

In an embodiment, the at least one genomic modification conferring reduced or loss of function comprises an insertion, a deletion, or a substitution.

In an embodiment, wherein the heterologous protein is a biologic drug. In an embodiment, the heterologous protein is an antibody. In an embodiment, the antibody is a human antibody or a humanized antibody. In an embodiment, the antibody is trastuzumab. In an embodiment, the heterologous protein is insulin. In an embodiment, the heterologous protein is a growth hormone. In an embodiment, the heterologous protein is a vaccine. In an embodiment, the heterologous protein is a therapeutic or diagnostic protein.

In an aspect, herein provided is a nucleic acid molecule comprising (a) a nucleic acid sequence according to SEQ ID NO: 2 or a reverse complement thereof, or (b) a functional variant of (a).

In an embodiment, the nucleic acid molecule is a short hairpin RNA (shRNA).

In an aspect, herein provided is a vector comprising the nucleic acid molecule herein disclosed.

In an aspect, herein provided is a use of a nucleic acid molecule herein disclosed or a vector herein disclosed for enhancing production of a heterologous protein in a plant or a plant cell.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments but should be given the broadest interpretation consistent with the description as a whole.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows a mass spectrometry-based OMICs workflow in an example embodiment of the disclosure. Bacterial samples grown in shake flask or bioreactor represent inoculum for N. benthamiana agroinfiltration. Plants are collected over a time course of infection (i.e. 0-7 days post infiltration (dpi)) and divided in two sample sets. One sample portion is subjected to protein extraction, digestion, labelling (TMTpro 16plex), purification and measurement on a high-resolution mass spectrometer. The second sample portion is subjected to metabolomic (MS/MS) profiling following metabolite extraction. Data analyses on the respective datasets was performed. Four biological and two technical bacterial replicates and eight biological plant replicates per experiment. Figure generated with Biorender.com.

FIGS. 2A-2C show a global proteome profiling of host-pathogen relationship leveraged for molecular pharming in an example embodiment of the disclosure. FIG. 2A) Diagram for number of identified proteins of Nicotiana benthamiana and Agrobacterium tumefaciens. FIG. 2B) Principal component analysis. Plant infiltrated with shake flask-grown A. tumefaciens, circle; bioreactor, square. FIG. 2C) Heatmap of normalized protein intensity using hierarchical clustering by Euclidian distance. Bacterial growth conditions prior to agroinfiltration defined as B=bioreactor, F=shake flask. Four biological and two technical bacterial replicates and eight biological plant replicates per experiment.

FIGS. 3A-3K show a differential proteome of N. benthamiana infiltrated with shake flask-versus bioreactor-grown A. tumefaciens in an example embodiment of the disclosure. FIGS. 3A-3D) Volcano plots of shake flask (right) and bioreactor (left)-infiltrated plants at 0 dpi (FIG. 3A), 1 dpi (FIG. 3B), 3 dpi (FIG. 3C), and 7 dpi (FIG. 3D). Significantly different N. benthamiana proteins (dark grey), A. tumefaciens (light grey), trastuzumab heavy chain (HC) and light chain (LC) (black). Student's t test P≤0.05; false discovery rate=0.01; S₀=1. FIG. 3E) Line plot of normalized trastuzumab intensity for flask and bioreactor (HC square, LC circle). Bioreactor datapoints at 1 dpi are identified by the dashed oval labelled “B”, and shake flask datapoints at 1 dpi are identified by the solid oval labelled “F”. Error bars represent standard deviations. Student's t test P≤0.05. FIG. 3F) Western blot for trastuzumab HC and LC abundance. FIG. 3G) Qualification of trastuzumab using Blitz system. Trastuzumab values presented as a normalized ratio to total protein concentration within the respective sample. 1 dpi, 3 dpi and 7 dpi are represented from left to right. Error bars represent standard deviations. FIGS. 3H-3I) Pie charts for percentage of significantly different N. benthamiana (FIG. 3H) and A. tumefaciens (FIG. 3I) proteins across all time points grouped by Gene Ontology Biological Processes (GOBP) unique to bioreactor (top; FIGS. 3H, 3I) or shake flask A. tumefaciens infiltration. (3J-3K) Scatter dot plots of normalized intensity of defence-related N. benthamiana proteins (FIG. 3J), and of normalized intensity of N. benthamiana apoplastic proteases (FIG. 3K) at 0 d, 1 dpi, 3 dpi and 7 dpi (F=shake flask, B=bioreactor-grown A. tumefaciens). Error bars represent standard deviations. Tukey's multiple comparisons test, *P≤0.05, ****P≤0.0001. Four biological and two technical bacterial replicates and eight biological plant replicates per experiment.

FIGS. 4A-4E show a time course proteome of N. benthamiana infiltrated with shake flask-grown A. tumefaciens in an example embodiment of the disclosure. FIG. 4A) Number of significantly different N. benthamiana proteins identified across the different timepoints: 1 dpi versus 0 dpi (grey=increased at 1 dpi, black=increased at 0 dpi), 3 dpi versus 0 dpi (grey=increased at 3 dpi, black=increased at 0 dpi), 7 dpi versus 0 dpi (grey=increased at 7 dpi, black=increased at 0 dpi). Student's t test, P<0.05; FDR=0.01; S₀=1. FIG. 4B) Line plot of normalized protein intensity for trastuzumab heavy chain (HC) and light chain (LC), and P19. Error bars represent standard deviations. FIG. 4C) One-dimensional (1D) annotation enrichment of Gene Ontology Biological Processes (GOBP). Student's t test P≤0.05; false discovery rate (FDR)=0.01; score<−0.5<0.5. FIG. 4D) Number of significantly different A. tumefaciens proteins identified across the different timepoints: 1 dpi versus 0 dpi (grey=increased at 1 dpi, black=increased at 0 dpi), 3 dpi versus 0 dpi (grey=increased at 3 dpi, black=increased at 0 dpi), 7 dpi versus 0 dpi (grey=increased at 7 dpi, black=increased at 0 dpi) dpi. Student's t test, P<0.05; FDR=0.01; So₀=1. FIG. 4E) Line plot of normalized protein intensity for virulence (VirH1, VirH2, VirD2,and VirB9) and motility (FlaA and FlaB) proteins. Error bars represent standard deviations. Four biological and two technical bacterial replicates and eight biological plant replicates per experiment.

FIGS. 5A-5E show the bioreactor time course proteome of N. benthamiana infiltrated with bioreactor-grown A. tumefaciens in an example embodiment of the disclosure. FIG. 5A) Number of significantly different N. benthamiana proteins identified across the different timepoints: 1 dpi versus 0 dpi (grey=increased at 1 dpi, black=increased at 0 dpi), 3 dpi versus 0 dpi (grey=increased at 3 dpi, black=increased at 0 dpi), 7 dpi versus 0 dpi (grey=increased at 7 dpi, black=increased at 0 dpi). Student's t test, P<0.05; FDR=0.01; S₀=1. FIG. 5B) Line plot of normalized protein intensity for trastuzumab heavy chain (HC) and light chain (LC) and P19. Error bars represent standard deviations. FIG. 5C) One-dimensional (1D) annotation enrichment of Gene Ontology Biological Processes (GOBP). Student's t test P≤0.05; FDR=0.01; score<−0.5<0.5. FIG. 5D) Number of significantly different A. tumefaciens proteins identified across different timepoints: 1 dpi versus 0 dpi (grey=increased at 1 dpi, black=increased at 0 dpi), 3 dpi versus 0 dpi (grey=increased at 3 dpi, black=increased at 0 dpi), 7 dpi versus 0 dpi (grey=increased at 7 dpi, black=increased at 0 dpi). Student's t test, P<0.05; FDR=0.01; S₀=1. FIG. 5E) Line plot of normalized protein intensity for virulence (VirH1, VirH2, VirD2, and VirB9) and motility (FlaA and FlaB) proteins. Vertical bars are (standard deviations) at a single timepoint. Four biological and two technical bacterial replicates and eight biological plant replicates per experiment.

FIG. 6 shows a differential metabolite profile of N. benthamiana infiltrated with shake flask-versus bioreactor-grown A. tumefaciens in an example embodiment of the disclosure. Metabolites identified by MS2 spectral matching and abundance statistically significant in at least one test. Student's t test, P-value<0.05; false discovery rate=0.01; S₀=1; t test difference refers to fold difference between the two indicated tests. *Significantly different P-value<0.05. Four biological and two technical bacterial replicates and eight biological plant replicates per experiment.
JA Precursor: Label A—12-Oxo phytodienoic acid
Purine: Label B—Adenine
Cofactor: Label C—Riboflavin
Aromatic:
Label D—Phenylacetaldehyde
Label E—7-Hydroxycoumarine

Label F—Feruloyl putrescine
Label G—Feruloyltyramine
Label H—Feruloyl O-methyldopamine
Label I—5-p-Coumaroylquinic acid
Terpene:
Label J—Myrcene
Label K—2(1H)-Naphthalenone, 4a,56788a-hexahydro-6-hydroxy-38-dimethyl-5-(1-methylethyl)—, (4aR,5S,6S,8R,8aS)—
Label L—Napththo[23-b]furan-2(4H)-one, 4a,56788a,99a-octahydro-9a-hydroxy-34a,5-trimethyl—, (4aR,5S,8aS,9aR)—
Label M—2-Butanone, 4-[3-(beta-D-glucopyranosyloxy)-4-hydroxy-266-trimethyl-1-cyclohexen-1-yl]—
Label N—2-Cyclohexen-1-one, 4- [(1E)-3-(beta-D-glucopyranosyloxy)-1-buten-1-yl]-4-hydroxy-355-trimethyl—, (4S)—

FIG. 7 shows a KEGG map integrating metabolomics and proteomics datasets. A-linolenic acid metabolism pathway generated with KEGG in an example embodiment of the disclosure. Jasmonic acid pathway highlighted in grey, identified metabolite node (12-OPDA) is highlighted, boxes highlighted contain identified proteins with corresponding abundance profiles to the metabolite.

FIGS. 8A-8B show trastuzumab production under aspartic protease shRNA knockdown in an example embodiment of the disclosure. Quantification of trastuzumab production (mg/kg) after 7 dpi. shRNA designed for aspartic proteinase A1-like; LOC107809122 (AP A1), aspartyl protease AED3-like; LOC107806163 (AED3-1), aspartyl protease AED3-like; LOC107828826 (AED3-2). Plants were co-infiltrated with the respective shRNA and pPFC0058 or only pPFC0058 for the control. FIG. 8A) Comparison of trastuzumab production between the control and single shRNA for AED3-1, AED3-2 and AP A1. Welch's t-test with *P≤0.05. FIG. 8B) Comparison of trastuzumab production between the control and double shRNA for AED3-1&2. Kruskal-Wallis test with ns=not significant. Samples measured in 10 biological and 3 technical replicates.

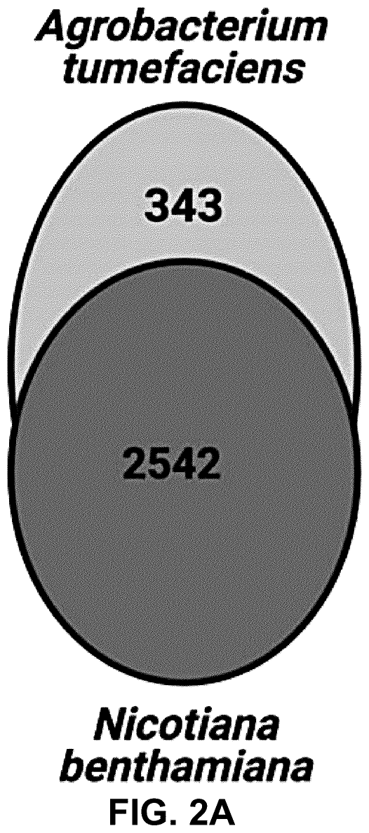

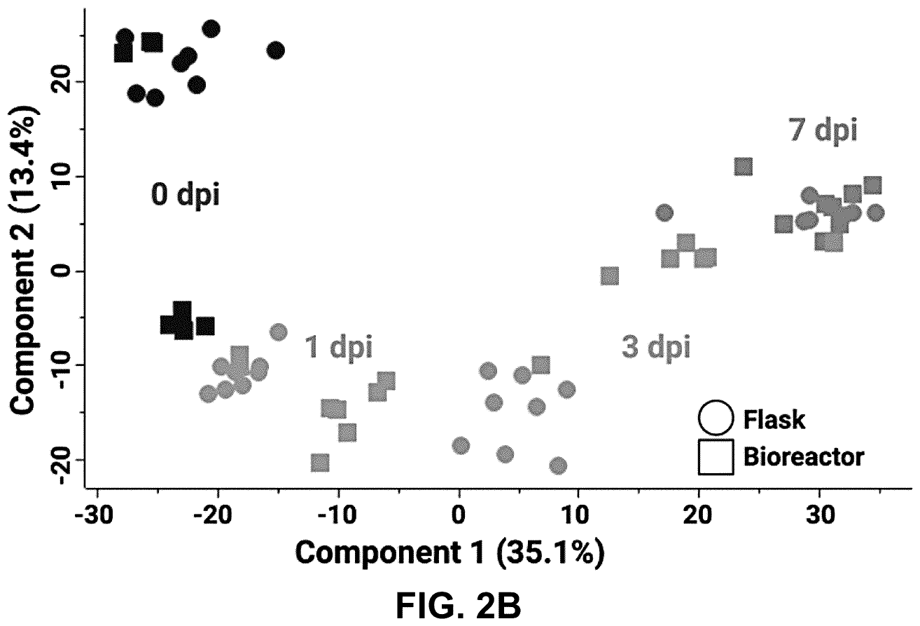

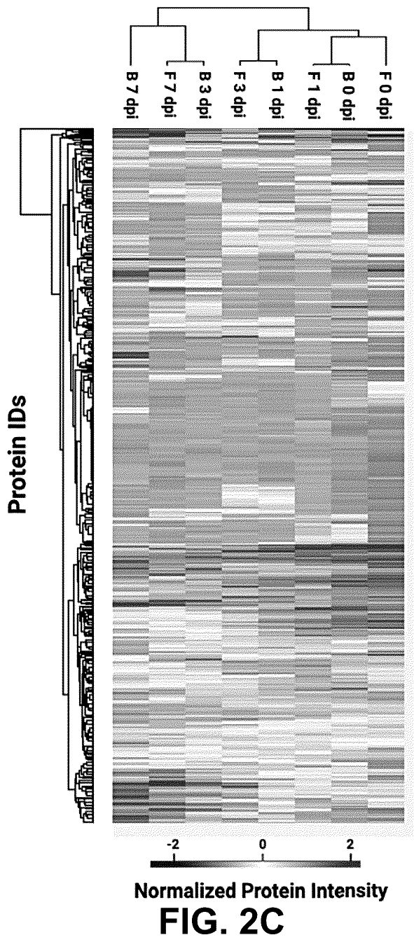

FIG. 9 shows a plot of PCA components following proteomic profiling of N. benthamiana plants infiltrated with shake flask versus bioreactor grown A. tumefaciens in an example embodiment of the disclosure. Component 1 (35.1%) and Component 2 (13.4%) are presented in FIG. 2B and account for time post infiltration and A. tumefaciens growth prior to agroinfiltration (shake flask vs bioreactor), respectively.

FIGS. 10A-10B show a western blot loading and quantification in an example embodiment of the disclosure. FIG. 10A) SDS-PAGE sample loading control for the western blot. Aliquots of undigested protein extract from the proteomic sample preparation were concentrated using Amicon Ultra-0.5 Centrifugal Filter Units (Millipore), separated by SDS-PAGE and stained with Coomassie blue to view protein concentrations prior to western blot. FIG. 10B) Relative quantification of trastuzumab western blot protein amounts using ImageJ.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments but should be given the broadest interpretation consistent with the description as a whole.

### DETAILED DESCRIPTION OF THE DISCLOSURE

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect or embodiment so defined may be combined with any other aspect or aspects or embodiment or embodiments unless clearly indicated to the contrary. In particular, any feature described herein may be combined with any other feature or features described herein.

### I. Definitions

As used herein, the following terms may have meanings ascribed to them below, unless specified otherwise. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this specification and the appended claims, the singular forms “a”, “an” and “the” include plural references unless the content clearly dictates otherwise. Thus, for example, a composition containing “a compound” includes a mixture of two or more compounds.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of’ or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either or both of those included limits are also included in the description. The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The term “nucleic acid” as used herein may refer to a biopolymer comprising monomers of nucleotides, such as deoxyribonucleic acid (DNA), ribonucleic acid (RNA) and other polynucleotides of modified nucleotides and/or nucleotide derivatives, and may be either double stranded (ds) or single stranded (ss). “Modified” bases include, for example, tritiated bases and unusual bases such as inosine. A variety of modifications can be made to DNA and RNA; thus “nucleic acid molecule”, “DNA molecule”, and “RNA molecule” embrace chemically, enzymatically, or metabolically modified forms. Examples of modified nucleotides which can be used to generate the nucleic acids disclosed herein include xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine or fluorophore and quencher conjugated nucleotides. Alternatively, the nucleic acid molecules can be produced biologically using an expression vector. In an embodiment, modified nucleotides comprise one or more modified bases (e.g. unusual bases such as inosine, and functional modifications to the bases such as amino modifications), modified backbones (e.g. peptide nucleic acid, PNA) and/or other chemically, enzymatically, or metabolically modified forms.

The term “functional variant” as used herein refers to a variant of the nucleic acid that retains the functional property of the nucleic acid, for example, the ability to prevent translation of a polypeptide, such as AED3-1.

It should also be understood that, in certain methods described herein that include more than one step or act, the order of the steps or acts of the method is not necessarily limited to the order in which the steps or acts of the method are recited unless the context indicates otherwise.

Although any methods and materials similar or equivalent to those described herein can also be used in the practice or testing of the present disclosure, examples of methods and materials are now described.

### II. Methods

Recently, there has been a growing interest in molecular pharming in plants, and specifically in developing new methods to increase the yield of heterologous proteins in plants. The inventors discovered that inhibiting the specific aspartic protease AED3-1, but not other aspartic proteases, increased the yield of a heterologous protein in plants, and thereby developed methods for enhancing the production of heterologous proteins in plants by inhibiting this protease.

Accordingly, in an aspect, provided herein is a method of enhancing production of a heterologous protein in a plant or plant cell comprising:
a) introducing into the plant or plant cell a nucleic acid molecule encoding the heterologous protein operably linked to a promoter;
b) inhibiting aspartic protease AED3-1; and
c) growing the plant or plant cell to obtain a plant that expresses the heterologous protein.

In an aspect, herein provided is a method of enhancing production of a heterologous protein in a plant or plant cell comprising:
a) introducing into the plant or plant cell a nucleic acid molecule encoding the heterologous protein operably linked to a promoter;
b) inhibiting a protein selected from Table 1 or Table 2; and
c) growing the plant or plant cell to obtain a plant that expresses the heterologous protein.

The term “heterologous protein”, as used herein means that the amino acid or nucleotide sequence encoding the protein is not native to the plant or the plant cell. The heterologous protein can be introduced into the plant or plant cell using an expression vector.

The term “enhancing production” or “enhanced production” of a heterologous protein as used herein means an increase in yield of the heterologous protein compared to a system wherein only the heterologous protein is introduced into the plant or the plant cell without inhibiting AED3-1. Yield can be measured, for example, as the relative proportion of the heterologous protein compared to total soluble protein in, for example a plant, plant part, or plant cell, or an extract derived therefrom. Yield can also be measured, for example, as the yield of the heterologous protein per unit area per unit time in a production facility. The term “plant part” refers to any part of a plant including but not limited to the embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen, and the like.

The term “introducing into the plant or plant cell” as used herein includes both the stable integration of a nucleic acid molecule into the genome of a plant cell to prepare a transgenic plant as well as the transient integration of a nucleic acid molecule into a plant or a plant cell.

The term “operatively linked” as used herein refers to an arrangement of two or more components, wherein the components so described are in a relationship permitting them to function in a coordinated manner. For example, a transcriptional regulatory sequence or a promoter is operably linked to a coding sequence if the transcriptional regulatory sequence or promoter facilitates aspects of the transcription of the coding sequence. Aspects of the transcription process include, but are not limited to, initiation, elongation, attenuation and termination. In general, an operably linked transcriptional regulatory sequence joined in cis with the coding sequence, but it is not necessarily directly adjacent to it.

The terms “AED3-1”, “aspartic protease AED3-1”, and “aspartic protease AED3-like” as used herein mean “aspartyl protease apoplastic enhanced disease susceptibility 3-1”. This protease is also known as “aspartic protease 53” or “AP53”. AED3-1 can be from any plant or plant cell, and optionally as shown in Uniprot Accession No. A0A1S4BAC6.

The term “inhibiting” as used herein refers to reducing or eliminating the expression and/or function of a polypeptide such as AED3-1. Inhibiting can be achieved through a variety of means including, for example, genomic modifications to the nucleic acid encoding the polypeptide, preventing the translation of the polypeptide from a messenger RNA encoding the polypeptide, and interfering with the activity of polypeptide when it is expressed. Inhibiting can also be achieved through a combination of such methods.

The phrase “growing a plant or plant cell to obtain a plant that expresses the heterologous protein” includes both growing transgenic plant cells into a mature plant as well as growing or culturing a mature plant that has received the nucleic acid molecules encoding the heterologous protein. One of skill in the art can readily determine the appropriate growth conditions in each case.

The term “plant” as used herein includes whole plants, progeny of the plants and plant parts, including seeds, siliques, fruit, leaves, flowers, shoots, stems, roots, plant cells, isolated plant cells, plant cell cultures, tissues and organs.

Further regulators of protein expression, such as suppressors of gene silencing, cis-regulatory elements like promoters, and trans-regulatory elements like transcription factor peptides, can be used with these methods to further enhance the production of the heterologous protein. Accordingly, in an embodiment, the method further comprises introducing into the plant or plant cell a nucleic acid molecule encoding a suppressor of gene silencing protein operably linked to a promoter.

In an embodiment, the suppressor of gene silencing protein is P19.

P19 is a viral protein that suppresses gene silencing. For example, P19 from Tomato Bushy Stunt Virus (TBSV; Genbank accession: M21958) is an example of a protein known to function as a potent suppressor of gene silencing in plants as well as in animals.

In an embodiment, the plant or plant cell expresses the P19 protein from TBSV. In an embodiment, the P19 protein from TBSV is expressed from a nucleic acid molecule which has been modified to optimize expression levels in Nicotiana plants. In a specific embodiment, the modified P19-encoding nucleic acid molecule has the sequence shown in SEQ ID NO: 6.

The P19 protein can be expressed from an expression vector comprising a single expression cassette or from an expression vector containing one or more additional cassettes, wherein the one or more additional cassettes comprise transgenic DNA encoding one or more heterologous proteins or ABCF4 proteins. An example of a P19 expression vector includes pPFC0058 (e.g., FIGS. 8A and 8B).

In an embodiment, the nucleic acid molecule encoding the heterologous protein and the nucleic acid molecule encoding the suppressor of gene silencing protein are introduced on separate vectors. In an embodiment, the nucleic acid molecule encoding the heterologous protein and the nucleic acid molecule encoding the suppressor of gene silencing protein are introduced on the same vector.

Plant genera and species suitable for use as the plant or plant cell in the present methods include, for example, genus Nicotiana, including Nicotiana benthamiana (N. benthamiana).

Accordingly, in an embodiment, the plant or plant cell is of genus Nicotiana. In an embodiment, the plant or plant cell is N. benthamiana.

In an embodiment, the AED3-1 comprises an amino acid sequence according to SEQ ID NO: 1.

The AED3-1 or P19 of the present disclosure may comprise a sequence with at least 60% sequence identity to the sequences described herein. In another example, the sequences may have at least 55%, 60%, 65%, 70%, 75%, 80%, 85%, 86%, 87%, 88%, 89%, 90%, 91%, 92%, 93%, 94%, 95%, 96%, 97%, 98%, 99%, or 100% sequence identity at the nucleic acid or amino acid level to sequences described herein. In yet another example, the sequences are complements or reverse complements to the sequences described herein. The proteins encoded by any variant sequences herein disclosed retain the activity and specificity of the proteins encoded by the reference sequences herein disclosed.

As used here, the term “sequence identity” refers to the percentage of sequence identity between two polypeptide sequences or two nucleotide sequences. To determine the percent identity of two amino acid sequences or of two nucleotide sequences, the sequences are aligned for optimal comparison purposes (e.g., gaps can be introduced in the sequence of a first amino acid or nucleotide d sequence for optimal alignment with a second amino acid or nucleotide sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions multiplied by 100%). In an embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul (1990), modified as in Karlin and Altschul (1993). Such an algorithm is incorporated into the NBLAST and XBLAST programs of Altschul et al. (1990). BLAST nucleotide searches can be performed with the NBLAST nucleotide program parameters set, e.g., for score=100, wordlength=12 to obtain nucleotide sequences homologous to a nucleic acid molecule of the present disclosure. BLAST protein searches can be performed with the XBLAST program parameters set, e.g., to score=50, wordlength=3 to obtain amino acid sequences homologous to a protein molecule of the present disclosure. To obtain gapped alignments for comparison purposes, Gapped BLAST can be utilized as described in Altschul et al. (1997). Alternatively, PSI-BLAST can be used to perform an iterated search which detects distant relationships between molecules (Altschul et al., 1997). When utilizing BLAST, Gapped BLAST, and PSI-BLAST programs, the default parameters of the respective programs (e.g., of XBLAST and NBLAST) can be used (see, e.g., the NCBI website). Another non-limiting example of a mathematical algorithm utilized for the comparison of sequences is the algorithm of Myers and Miller (1988). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In an embodiment, the AED3-1 protein comprises a sequence with at least 55%, 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO: 1. In an embodiment, the AED3-1 protein comprises a sequence with at least 55% sequence identity to SEQ ID NO: 1. In an embodiment, the AED3-1 protein comprises a sequence with at least 60% sequence identity to SEQ ID NO: 1. In an embodiment, the AED3-1 protein comprises a sequence with at least 65% sequence identity to SEQ ID NO: 1. In an embodiment, the AED3-1 protein comprises a sequence with at least 70% sequence identity to SEQ ID NO: 1. In an embodiment, the AED3-1 protein comprises a sequence with at least 75% sequence identity to SEQ ID NO: 1. In an embodiment, the AED3-1 protein comprises a sequence with at least 80% sequence identity to SEQ ID NO: 1. In an embodiment, the AED3-1 protein comprises a sequence with at least 85% sequence identity to SEQ ID NO: 1. In an embodiment, the AED3-1 protein comprises a sequence with at least 86% sequence identity to SEQ ID NO: 1. In an embodiment, the AED3-1 protein comprises a sequence with at least 87% sequence identity to SEQ ID NO: 1. In an embodiment, the AED3-1 protein comprises a sequence with at least 88% sequence identity to SEQ ID NO: 1. In an embodiment, the AED3-1 protein comprises a sequence with at least 89% sequence identity to SEQ ID NO: 1. In an embodiment, the AED3-1 protein comprises a sequence with at least 90% sequence identity to SEQ ID NO: 1. In an embodiment, the AED3-1 protein comprises a sequence with at least 91% sequence identity to SEQ ID NO: 1. In an embodiment, the AED3-1 protein comprises a sequence with at least 92% sequence identity to SEQ ID NO: 1. In an embodiment, the AED3-1 protein comprises a sequence with at least about 93% sequence identity to SEQ ID NO: 1. In an embodiment, the AED3-1 protein comprises a sequence with at least 94% sequence identity to SEQ ID NO: 1. In an embodiment, the AED3-1 protein comprises a sequence with at least 95% sequence identity to SEQ ID NO: 1. In an embodiment, the AED3-1 protein comprises a sequence with at least 96% sequence identity to SEQ ID NO: 1. In an embodiment, the AED3-1 protein comprises a sequence with at least 97% sequence identity to SEQ ID NO: 1. In an embodiment, the AED3-1 protein comprises a sequence with at least 98% sequence identity to SEQ ID NO: 1. In an embodiment, the AED3-1 protein comprises a sequence with at least 99% sequence identity to SEQ ID NO: 1.

In an embodiment, the nucleic acid molecule encoding the P19 suppressor of gene-silencing protein comprises a sequence with at least 70%, 75%, 80%, 80%, 85%, 90%, 95%, 98%, 99% or 100% sequence identity to SEQ ID NO:6.

In an embodiment of the method or modified plant or plant cell herein disclosed, the sequence identity is over the entire length of the amino acid sequence In an embodiment, the inhibiting in b) comprises contacting the plant or plant cell with an inhibitor of AED3-1.

The term “inhibitor of AED3-1” as used herein refers to any molecule that inhibits the expression and/or function of AED3-1. Said inhibitor can comprise a nucleic acid, for example an RNA interference (RNAi) nucleic acid such as a short hairpin RNA (shRNA), a small interfering RNA (siRNA), a micro RNA (miRNA), or an antisense oligonucleotide (ASO) that prevents translation of the AED3-1 protein from a messenger RNA (mRNA) encoding the AED3-1 protein. Said inhibitor can also comprise, for example, a peptide or small molecule that inhibits the function of AED3-1 protein.

Accordingly, in an embodiment, the inhibitor comprises a polypeptide and/or a small molecule drug. In an embodiment, the inhibitor comprises a polypeptide. In an embodiment, the inhibitor comprises a small molecule drug.

In an embodiment, the inhibitor comprises a short hairpin RNA (shRNA), a small interfering RNA (siRNA), a micro RNA (miRNA), or an antisense oligonucleotide (ASO). In an embodiment, the inhibitor comprises an shRNA. In an embodiment, the inhibitor comprises an siRNA. In an embodiment, the inhibitor comprises an miRNA. In an embodiment, the inhibitor comprises an ASO.

The inventors created an shRNA against AED3-1 that, when used, increased expression of a heterologous protein in a plant. This shRNA has a sequence according to SEQ ID NO: 2. Other shRNAs for inhibiting AED3-1 can be engineered, for example, by identifying possible target sequences using the cDNA of AED3-1, annotating the sequences to add a hairpin loop and a reverse complementary sequence to allow the transcript to form the desired shRNA structure. Restriction sites can also be added to prepare the sequence to be inserted into a plasmid vector. Some variability within an shRNA sequence can be tolerated and will still inhibit translation of AED3-1, for example 1, 2, or 3 nucleotide substitutions.

Accordingly, in an embodiment, the inhibitor comprises a nucleic acid sequence according to SEQ ID NO: 2 or a functional variant thereof.

In an embodiment, the inhibiting in b) comprises introducing into the plant or plant cell a nucleic acid molecule encoding an inhibitor of AED3-1 herein disclosed operably linked to a promoter.

In an embodiment, the inhibiting in b) comprises introducing into the plant or plant cell at least one genomic modification in a gene encoding the AED3-1, wherein the at least one genomic modification confers reduced or loss of function of the AED3-1 as compared to a plant of the same species lacking the at least one genomic modification.

The term “reduced . . . function” as used herein refers to a reduction of at least 50%, at least 55%, at least 60%, at least 65%, at least 70%, at least 75%, at least 80%, at least 85%, at least 90%, at least 95%, at least 96%, at least 97%, at least 98%, or at least 99% in AED3-1 protein activity compared to a plant of the same species lacking the at least one genomic modification.

In an embodiment of the method or modified plant or plant cell herein disclosed, the reduced function comprises a reduction of at least 50%, at least 55%, at least 60%, at least 65%, at least 70%, at least 75%, at least 80%, at least 85%, at least 90%, at least 95%, at least 96%, at least 97%, at least 98%, or at least 99% in AED3-1 protein activity compared to a plant the same species lacking the at least one genomic modification. In an embodiment of the method or modified plant or plant cell herein disclosed, the reduced function comprises a reduction of at least 50% in AED3-1 protein activity compared to a plant the same species lacking the at least one genomic modification. In an embodiment of the method or modified plant or plant cell herein disclosed, the reduced function comprises a reduction of at least 55% in AED3-1 protein activity compared to a plant the same species lacking the at least one genomic modification. In an embodiment of the method or modified plant or plant cell herein disclosed, the reduced function comprises a reduction of at least 60% in AED3-1 protein activity compared to a plant the same species lacking the at least one genomic modification. In an embodiment of the method or modified plant or plant cell herein disclosed, the reduced function comprises a reduction of at least 65% in AED3-1 protein activity compared to a plant the same species lacking the at least one genomic modification. In an embodiment of the method or modified plant or plant cell herein disclosed, the reduced function comprises a reduction of at least 70% in AED3-1 protein activity compared to a plant the same species lacking the at least one genomic modification. In an embodiment of the method or modified plant or plant cell herein disclosed, the reduced function comprises a reduction of at least 75% in AED3-1 protein activity compared to a plant the same species lacking the at least one genomic modification. In an embodiment of the method or modified plant or plant cell herein disclosed, the reduced function comprises a reduction of at least 80% in AED3-1 protein activity compared to a plant the same species lacking the at least one genomic modification. In an embodiment of the method or modified plant or plant cell herein disclosed, the reduced function comprises a reduction of at least 85% in AED3-1 protein activity compared to a plant the same species lacking the at least one genomic modification. In an embodiment of the method or modified plant or plant cell herein disclosed, the reduced function comprises a reduction of at least 90% in AED3-1 protein activity compared to a plant the same species lacking the at least one genomic modification. In an embodiment of the method or modified plant or plant cell herein disclosed, the reduced function comprises a reduction of at least 95% in AED3-1 protein activity compared to a plant the same species lacking the at least one genomic modification. In an embodiment of the method or modified plant or plant cell herein disclosed, the reduced function comprises a reduction of at least 96% in AED3-1 protein activity compared to a plant the same species lacking the at least one genomic modification. In an embodiment of the method or modified plant or plant cell herein disclosed, the reduced function comprises a reduction of at least 97% in AED3-1 protein activity compared to a plant the same species lacking the at least one genomic modification. In an embodiment of the method or modified plant or plant cell herein disclosed, the reduced function comprises a reduction of at least 98% in AED3-1 protein activity compared to a plant the same species lacking the at least one genomic modification. In an embodiment of the method or modified plant or plant cell herein disclosed, the reduced function comprises a reduction of at least 99% in AED3-1 protein activity compared to a plant the same species lacking the at least one genomic modification.

In an embodiment of the method or modified plant or plant cell herein disclosed, the at least one genomic modification comprises an insertion, a deletion, and/or a substitution. In an embodiment of the method or modified plant or plant cell herein disclosed, the at least one genomic modification comprises an insertion. In an embodiment of the method or modified plant or plant cell herein disclosed, the at least one genomic modification comprises a deletion. In an embodiment of the method or modified plant or plant cell herein disclosed, the at least one genomic modification comprises a substitution.

The genomic modification that confers reduced or loss of function can, for example, be one that reduces or eliminates gene expression and/or one that results in a protein that is no longer capable or has diminished capacity to perform its native role, for example a truncated protein lacking a catalytic domain.

The method for introducing into the plant or plant cell at least one genomic modification can comprise targeted genome modification, for example using CRISPR (Clustered Regularly Interspaced Short Palindromic Repeats), CRISPR-associated (Cas) gene (CRISPR/Cas)-based methods, transcription activator-like effector nuclease (TALEN)-based methods, or zinc-finger nuclease (ZFN)-based methods.

CRISPR-based gene editing involves an endonuclease such as a Cas enzyme to cut DNA and a guide RNA (gRNA) which directs the endonuclease to the exact spot in the genome where a cut is to be made. Once the DNA is cut, the cell's repair mechanisms can be harnessed, for example, to add or delete nucleotides, allowing for targeted modifications. TALENs are restriction enzymes that can be engineered to cut specific sequences of DNA. They include a TAL effector DNA-binding domain, which contains a repeated highly conserved 33-34 amino acid sequence with divergent 12th and 13th amino acids, which referred to as the Repeat Variable Diresidue (RVD), are highly variable and show a strong correlation with specific nucleotide recognition. This specific DNA recognition allows for the engineering of specific DNA-binding domains by selecting a combination of repeat segments containing the appropriate RVDs. Once bound on the target DNA region, a DNA cleavage domain with nuclease activity in TALEN can cut DNA strands. The TALEN can be introduced into cells, for use in gene editing or for genome editing in situ. ZFNs are artificial restriction enzymes generated by fusing a zinc finger DNA-binding domain to a DNA-cleavage domain. The DNA-binding domains of individual ZFNs typically contain between three and six individual zinc finger repeats and can each recognize between 9 and 18 base pairs. This feature allows one to engineer the Zinc finger domains to target specific desired DNA sequences, which enables zinc-finger nucleases to target unique sequences within complex genomes.

Accordingly, in an embodiment, the method comprises targeted genome modification.

In an embodiment, the inhibiting in b) further comprises introducing a guide RNA (gRNA) into the plant or plant cell, wherein the gRNA is complementary to a nucleic acid target sequence within the gene encoding the AED3-1 protein.

In an embodiment, the gRNA is a single guide RNA (sgRNA).

The term “target sequence” as used herein refers to a nucleic acid sequence within the AED3-1 gene that is complementary to the gRNA, and that comprises a site at which targeted genome modification occurs, wherein the genome modification can result in reduced or loss of function of AED 3-1 as compared to a plant or plant cell of the same species lacking the modification.

The gRNA can be expressed from an expression vector comprising the nucleic acid target sequence operably linked to a promoter. This expression vector can further comprise a nucleic acid encoding an endonuclease such as Cas9 or Cas12 operably linked to a promoter. Alternatively, the nucleic acid encoding the endonuclease can be operably linked to a promoter and comprised in a separate expression vector. Further, the gRNA, the endonuclease (or a nucleic acid encoding the endonuclease), or both can be directly transformed into the plant without use of vectors, such as by particle bombardment, polyethylene glycol (PEG) induced transformation, or nanoparticle mediated transformation.

Accordingly, in an embodiment, the introducing the gRNA into the plant or plant cell comprises an expression vector comprising a nucleic acid target sequence complementary to the gRNA, wherein the nucleic acid target sequence is operably linked to a promoter herein disclosed. In an embodiment, the expression vector further comprises a promoter operably linked to a nucleic acid encoding an endonuclease.

In an embodiment, the introducing in b) further comprises introducing into the plant or plant cell a nucleic acid encoding an endonuclease operably linked to a promoter.

In an embodiment, the endonuclease is a Cas9 or a Cas12 endonuclease. In an embodiment, the endonuclease is a Cas9 endonuclease. In an embodiment, the endonuclease is a Cas12 endonuclease. In an embodiment, the endonuclease is a Casφ endonuclease. In an embodiment, the endonuclease is a CasMINI endonuclease.

In an embodiment, the endonuclease is a plant optimized endonuclease.

The term “promoter” as used herein refers to a nucleotide sequence that directs the transcription of a gene or coding sequence to which it is operably linked.

The promoter can be any promoter suitable for driving the expression of a heterologous protein, an inhibitor such as, for example, an shRNA, a gRNA, or an endonuclease herein disclosed, as appropriate. For example, promoters suitable for driving the expression of a heterologous protein or an endonuclease can comprise an inducible promoter, or a constitutive promoter such as the ubiquitin promoter, the actin (Act) promoter, or the cauliflower mosaic virus 35S (CaMV35S) promoter. Promoters suitable for driving a gRNA can include, for example, small RNA promoters (SP) such as Arabidopsis thaliana U6 or U3 promoters, including the U6-26p promoter.

In an embodiment, the promoter operably linked to the nucleic acid encoding the heterologous protein is a ubiquitin promoter, an Act promoter, a CaMV35S promoter or an EC1.2 promoter. In an embodiment, the promoter operably linked to the nucleic acid encoding the heterologous protein is a ubiquitin promoter. In an embodiment, the promoter operably linked to the nucleic acid encoding the heterologous protein is an Act promoter. In an embodiment, the promoter operably linked to the nucleic acid encoding the heterologous protein a CaMV35S promoter.

In an embodiment, the promoter operably linked to the nucleic acid encoding the endonuclease is a ubiquitin promoter, an Act promoter, a CaMV35S promoter or an EC1.2 promoter. In an embodiment, the promoter operably linked to the nucleic acid encoding the endonuclease is a ubiquitin promoter. In an embodiment, the promoter operably linked to the nucleic acid encoding the endonuclease is an Act promoter. In an embodiment, the promoter operably linked to the nucleic acid encoding the endonuclease a CaMV35S promoter.

As used herein, the term “vector” or “expression vector” means a nucleic acid molecule, such as a plasmid, comprising regulatory elements and a site for introducing transgenic DNA, which is used to introduce said transgenic DNA into a plant or plant cell. The transgenic DNA can encode a heterologous protein, which can be expressed in and isolated from a plant or plant cells. Regulatory elements can include promoters, 5′ and 3′ untranslated regions (UTRs), terminator sequences or truncations thereof, Kozak boxes, TATA boxes, CAAT boxes and/or one or more enhancers. Vectors useful in the present methods are well known in the art. In an embodiment, the vector is a commercially-available vector.

In an embodiment, the expression vector is comprised in Agrobacterium tumefaciens.

The method for introducing into the plant or plant cell at least one genomic modification can also comprise physically-induced mutagenesis or chemically-induced mutagenesis. Physically-induced mutagenesis can include, for example, methods that cause irradiation, such as X-ray, gamma rays, neutrons, beta and alpha particles, protons and ion beams. Chemically-induced mutagenesis can include treatment with diverse chemicals such as alkylating agents (e.g., 1-methyl-1-nitrosourea (MNU), 1-ethyl-1-nitrosourea (ENU), methyl methanesulphonate (MMS), ethyl methanesulphonate (EMS), dimethyl sulphate (DMS), diethyl sulphate (DES), 1-methyl-2-nitro-1-nitrosoguanidine (MNNG), 1-ethyl-2-nitro-1-nitrosoguanidine (ENNG); N,N-dimethylnitrous amide (NDMA), and N,N-diethylnitrous amide (NDEA)), azides (e.g., sodium azide), hydroxylamine, antibiotics (e.g., actinomycin D, mitomycin C, azaserine, or streptonigrin), nitrous acid, acridine orange, and base analogues (e.g., 5-bromouracil (5-BU), maleic hydrazide, 5-bromodeoxyuridine, and 2-aminopurine (2AP)).

In an embodiment, the introducing into the plant or plant cell the at least one genomic modification comprises physically-induced mutagenesis. In an embodiment, the introducing into the plant or plant cell the at least one genomic modification comprises chemically-induced mutagenesis. In an embodiment, the chemically-induced mutagenesis comprises an alkylating agent, an azide, hydroxylamine, an antibiotic, nitrous acid, acridine orange, a base analogue or a combination thereof. In an embodiment, the chemically-induced mutagenesis comprises an alkylating agent. In an embodiment, the chemically-induced mutagenesis comprises MNU, ENU, MMS, EMS, DMS, DES, MNNG, ENNG; NDMA, NDEA, or a combination thereof. In an embodiment, the chemically-induced mutagenesis comprises ethyl methanesulfonate (EMS). In an embodiment, the chemically-induced mutagenesis comprises DMS. In an embodiment, the chemically-induced mutagenesis comprises DES. In an embodiment, the chemically-induced mutagenesis comprises MNNG. In an embodiment, the chemically-induced mutagenesis comprises ENNG. In an embodiment, the chemically-induced mutagenesis comprises NDMA. In an embodiment, the chemically-induced mutagenesis comprises NDEA.

In an embodiment, the enhanced production of the heterologous protein is transient.

In an embodiment, the heterologous protein is a biologic drug.

In an embodiment, the heterologous protein is an antibody or an antibody fragment.

As used herein, the term “antibody” refers to an immunoglobulin (Ig) molecule and immunologically active portions of an immunoglobulin molecule, i.e., molecules that contain an antigen binding site that specifically binds (immunoreacts with) an antigen. By “specifically bind”, “immunoreacts with”, or “directed against” is meant that the antibody reacts with one or more antigenic determinants of the desired antigen and does not react with other polypeptides or binds at much lower affinity (K_{d}>10⁻⁶). Antibodies include, but are not limited to, polyclonal antibodies, monoclonal antibodies, chimeric antibodies and humanized antibodies. The antibody may be from recombinant sources and/or produced in transgenic animals or plants.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

An “antibody fragment” as used herein may include any suitable antigen- binding fragment known in the art. The term “antibody fragment” includes, without limitation, Fv (a molecule comprising the VL and VH), single chain Fv (scFV; a molecule comprising the VL and VH connected by a peptide linker, Fab, Fab′, F (ab′)₂, dsFv, ds-scFv, single domain antibodies (sdAB; molecules comprising a single variable domain and 3 CDR), and multivalent presentations of these. Also included are dimers, minibodies, diabodies, nanobodies, and multimers thereof, and bispecific antibody fragments. The antibody fragment of the present disclosure may be obtained by manipulation of a naturally occurring antibody (such as, but not limited to) enzymatic digestion, or may be obtained using recombinant methods.

In general, antibody molecules obtained from humans relate to any of the classes IgG, IgM, IgA, IgE and IgD, which differ from one another by the nature of the heavy chain present in the molecule. Certain classes have subclasses as well, such as IgG1, IgG2 (further divided into IgG2a and IgG2b), IgG3 and IgG4. Furthermore, in humans, the light chain may be a kappa chain or a lambda chain. Other suitable antibody molecules can include camelid antibodies such as camelid VHH antibodies, chicken antibodies such as IgY antibodies, or humanized versions of VHH antibodies and IgY antibodies.

Accordingly, in an embodiment, the antibody disclosed herein is an IgG antibody, optionally an IgG1 antibody.

In an embodiment, the antibody is an IgG, IgM, IgA, IgE, or IgD antibody. In an embodiment the antibody is a IgM antibody. In an embodiment the antibody is a IgA antibody. In an embodiment the antibody is a IgE antibody. In an embodiment the antibody is a IgD antibody. In an embodiment the antibody is a IgG2 antibody, optionally IgG2a or IgG2b). In an embodiment the antibody is a IgG3 antibody. In an embodiment the antibody is a IgG4 antibody. In an embodiment, the antibody is a VHH antibody. In an embodiment, the antibody is an IgY antibody.

Examples of antibodies contemplated for use in the methods described herein include, but are not limited to, abciximab, adalimumab, alemtuzumab, basiliximab, belimumab, bevacizumab, brentuximab vedotin, canakinumab, certolizumab, cetuximab, daclizumab, daratumumab, denosumab, eculizumab, efalizumab, golimumab, ibritumomab tiuxetan, infliximab, ipilimumab, muromonab-CD3, natalizumab, nivolumab, ofatumumab, omalizumab, palivizumab, panitumumab, pembrolizumab, rituximab, tocilizumab, atlizumab, tositumomab, trastuzumab and ustekinumab.

Accordingly, in an embodiment, the antibody or antibody fragment is a human antibody or antibody fragment or a humanized antibody or antibody fragment. In an embodiment, the antibody is Trastuzumab or a modified form thereof, consisting of 2 heavy chains (HC) and 2 light chains (LC).

Trastuzumab (Herceptin® Genentech Inc., San Francisco, CA) is a humanized murine immunoglobulin G1K antibody that is used in the treatment of metastatic breast cancer.

Also contemplated are anti-epitope antibodies, including, but not limited to, anti-polyhistidine antibody, Penta-his antibody, anti-c-myc antibody, anti-myc antibody, anti-HA antibody, anti-hemagglutinin antibody, anti-FLAG antibody and anti-QCRL-1 antibody. In an embodiment, the protein is a serum or plasma protein such as a transport protein, regulatory protein, enzyme, protease inhibitor, clotting factor, lectin or globulin. Specific examples of these are alpha 1 antitrypsin, alpha 1 acid glycoprotein, alpha 1 fetoprotein, alpha2-macroglobulin, gamma globulins, beta-2 microglobulin, haptoglobin, ceruloplasmin, complement proteins, C-reactive protein (CRP), lipoproteins, transferrin, fibrinogen, prothrombin, thrombin, hemopexin, butyrylcholinesterase, acetylcholinesterase and plasma cholinesterases.

In an embodiment, the heterologous protein is insulin.

In other antibodies, the heterologous protein is a growth hormone.

In an embodiment, the heterologous protein is a vaccine.

In an embodiment, the heterologous protein is a therapeutic or diagnostic protein.

The term “therapeutic or diagnostic protein” as used herein refers to any peptide or protein, or fragments and/or fusion proteins thereof, that is known to be useful for the prevention, treatment, or amelioration of a condition, disease or disorder or for the diagnosis of any condition disease or disorder. The therapeutic or diagnostic protein can, for example, be a tissue protein such as collagen, vimentin, or laminin. The therapeutic or diagnostic protein can also be a protein useful as a vaccine, for example coronavirus spike protein, optionally beta coronavirus spike protein, or an HIV glycoprotein, optionally GP120. The therapeutic or diagnostic protein can also be a lectin, optionally Griffithsin.

The inventors have also discovered that growing Agrobacterium tumefaciens (A. tumefaciens) comprising the expression vector or vectors in a bioreactor prior to agroinfiltration of the plant leads to at least a transient increase in heterologous protein expression compared to growing the A. tumefaciens in a shake flask.

Accordingly, in an embodiment, the introducing into the plant comprises agroinfiltration. In an embodiment, the agroinfiltration comprises A. tumefaciens. In an embodiment, the A. tumefaciens is grown in a bioreactor prior to agroinfiltration.

In an embodiment, the heterologous protein is harvested less than 2 days, less than 3 days, less than 4 days, less than 5 days, less then 6 days, less than 7, less than 8 days, less than 9 days, or less than 10 days post infiltration (dpi). In an embodiment, the heterologous protein is harvested less than 2 dpi. In an embodiment, the heterologous protein is harvested less than 3 dpi. In an embodiment, the heterologous protein is harvested less than 4 dpi. In an embodiment, the heterologous protein is harvested less than 5 dpi. In an embodiment, the heterologous protein is harvested less than 6 dpi. In an embodiment, the heterologous protein is harvested less than 7 dpi. In an embodiment, the heterologous protein is harvested less than 8 dpi. In an embodiment, the heterologous protein is harvested less than 9 dpi. In an embodiment, the heterologous protein is harvested less than 10 dpi. In an embodiment, the heterologous protein is harvested about 3 to about 7 dpi. In an embodiment, the heterologous protein is harvested about 3 dpi.

### III. Plants and Plant cells

In an aspect, herein provided is a modified plant or plant cell with enhanced production of a heterologous protein, comprising:
a) a nucleic acid molecule encoding the heterologous protein operably linked to a promoter; and
b) at least one of the group consisting of:
   i) an inhibitor of aspartic protease AED3-1; and
   ii) at least one genomic modification in a gene encoding aspartic protease AED3-1 conferring reduced or loss of function of the AED3-1 as compared to a plant of the same species lacking the at least one genomic modification.

In an aspect, herein provided is a modified plant or plant cell with enhanced production of a heterologous protein, comprising:
a) a nucleic acid molecule encoding the heterologous protein operably linked to a promoter; and
b) at least one of the group consisting of:
   i) an inhibitor of a target protein selected from Table 1 or Table 2; and
   ii) at least one genomic modification in a gene encoding the target protein conferring reduced or loss of function of the target protein as compared to a plant of the same species lacking the at least one genomic modification.

In an embodiment, the modified plant or plant cell is produced by a method herein disclosed.

In an aspect, herein provided is a modified plant or plant cell with enhanced production of a heterologous protein, wherein the modified plant or plant cell is produced by a method herein disclosed.

### IV. Nucleic acids, Vectors, and Uses

In an aspect, herein provided is a nucleic acid molecule comprising (a) a nucleic acid sequence according to SEQ ID NO: 2 or a reverse complement thereof, or (b) a functional variant of (a).

In an embodiment, the nucleic acid molecule is an shRNA.

Also provided, in an aspect, is a vector comprising a nucleic acid molecule comprising (a) the nucleic acid sequence according to SEQ ID NO: 2 or the reverse complement thereof, or (b) the functional variant of (a).

In an aspect, herein provided is a use of a nucleic acid molecule herein disclosed or a vector herein disclosed, for enhancing production of a heterologous protein in a plant or plant cell.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

### EXAMPLES

The following non-limiting Examples are illustrative of the present disclosure:

### Example 1

To modulate the plant defence system, each stop of the molecular pharming pipeline requires careful consideration. For example, cultivation of A. tumefaciens is traditionally performed in shake flasks, however, limitations in production volume and strict control of growth parameters lead to batch inconsistencies and difficulties meeting good manufacturing practices (GMP) standards (Houdelet et al., 2017). Alternative bacterial growth strategies, which support more reliable GMPs, include microbial bioreactors, enabling the production of large quantities of bacteria in batch culture while promoting the fine tuning of growth parameters, including pH, oxygen concentration and temperature (Spiegel et al., 2019; Suzuki et al., 2006). For instance, the examination of A. tumefaciens bioreactor batch fermentation revealed maximum biomass concentrations, specific growth rates and elevated transient protein production levels (Leth and McDonald, 2017). Additionally, it was recently demonstrated that proteome remodelling of A. tumefaciens cultured under shake flask versus bioreactor conditions occurs, suggesting priming of the bacterium for plant cell invasion associated with bioreactor growth (Prudhomme et al., 2021a). Moreover, assessment of bacterial proteome alterations during the next critical step in target protein production (i.e. agroinfiltration) revealed increased bacterial mobility and enhanced virulence-associated protein production over a time course of exposure to agroinfiltration media (Prudhomme et al., 2021b). These studies showcase the feasibility and value of bioreactor bacterial growth for enhanced virulence, which may influence the plant defence response, impacting production of the target protein.

Mass spectrometry-based proteomics and metabolomics are powerful and adaptable tools used for exploring the global impact of growth conditions and environmental parameters on cellular processes (Aebersold and Mann, 2016). These methodologies provide robust and unbiased measurements of the abundance of proteins and metabolites within a biological sample, which define changes to the intra-and extracellular environment of both host and pathogen, along with strategies to integrate these data sets (Meissner et al., 2022; Smits and Vermeulen, 2016). To date, the applications of proteomics and metabolomics to address challenges and areas for improvement in molecular pharming are limited (Geddes-McAlister et al., 2022; Grosse-Holz et al., 2018a, 2018b); however, the potential to optimize target protein production through regulation of host response is tangible and within reach with such innovative approaches.

In this study, the state-of-the-art mass spectrometry-based proteomics was used combined with tandem mass tag profiling to define temporal changes in protein production of N. benthamiana infiltrated with A. tumefaciens grown in either shake flasks or bioreactors prior to agroinfiltration. The results of this study identify novel proteome remodelling in both the bacterial and plant systems regulated by bacterial growth conditions with the magnitude and duration of host defence significantly different during early timepoints. Here, the proteomics profiling monitors production of the heavy and light chains of trastuzumab, a human antibody that binds to HER2 (human epidermal growth factor receptor 2) for lysis of cancerous cells through antibody-dependent cellular cytotoxicity for metastatic breast cancer therapy (Grohs et al., 2010; Mclean, 2017). Moreover, the integration of proteomic profiling with metabolomics-based small molecule detection and quantification reveals how secondary metabolites fluctuate over time, providing new insight into the complexities of dual system modulation in molecular pharming. The discoveries of this study were validated through silencing of a select aspartic protease and exhibit increased trastuzumab production. Overall, our results indicate that bacterial growth in a bioreactor supports evasion of early plant defence responses towards A. tumefaciens and influences production of the heavy and light chains of trastuzumab at early time points following agroinfiltration. Such discoveries identify the potential for combining bioreactor bacterial growth with rapid tissue collection for increased target protein outputs within the molecular pharming industry. Furthermore, the potential for genetic manipulation of specific targets revealed through proteomic profiling to suppress host defences and increase recombinant protein production in molecular pharming is defined.

### Methods

### Bacterial Strains and Culture Conditions

Culturing of bacterial strains was performed as previously described (Prudhomme et al., 2021a). A modified version of the A. tumefaciens strain EHA105 (transformed with the T-DNA vector pPFC0058 for expression of the monoclonal antibody, trastuzumab and tombusvirus RNA silencing suppressor, P19) was used for the experiments (Garabagi et al., 2012a; Hood et al., 1993; Mclean, 2017). Bacteria were maintained on lysogeny broth (LB) agar plates (Fisher BioReagents, Ottawa, Ontario, Canada) at 28° C. supplemented with 0.1% kanamycin sulphate solution (50 mg/mL) and 0.1% rifampicin solution (25 mg/mL) to maintain plasmids.

### Bacterial Growth: Shake Flask and Bioreactor Conditions

Bacterial growth in shake flasks and bioreactors was performed as previously described (Prudhomme et al., 2021b). From the cultures of A. tumefaciens EHA105 (pPFC0058) maintained on LB agar plates, a single colony was selected and inoculated into 5 mL of liquid LB at 28° C. and 220 rpm and cultured overnight with kanamycin (50 μg/mL) and rifampicin (25 μg/mL). Subculturing (1/100) was performed in 1-L shake flasks or in bioreactor vessels (Sartorius, Göttingen, Germany) containing 500 mL of LB with kanamycin (50 μg/mL) and rifampicin (25 μg/mL) at pH 7 and 28°° C. with 170 rpm shaking for 8 h (shake flasks) or 600 rpm stirring with bubbling air flow for 12 h (bioreactors) (OD₆₀₀ₙₘ=1.4-1.5). For each growth condition, bacteria were grown to comparable optical densities and used for agroinfiltration. Colony-forming unit (CFU) counts were performed at the collection point (OD₆₀₀ₙₘ=1.4-1.5) with bacteria grown on LB agar plats at 28° C. for 24 h. Four biological replicates and two technical replicates ofA. tumefaciens from shake flask or bioreactor were used for plant infiltration.

### Pre-Infiltration Plant Growth

Nicotiana benthamiana KDFX (U.S. Pat. No. 11,499,160) seeds were sown in Sungro Horticulture Sunshine Professional Mix SS #4 with PlantProd fertilizer 20-8-20, 250 ppm, pH 6.0, Electrical Conductivity 2.5. Seeds were germinated in 200 cell trays with clear dome covers, transplanted to 3.5 sq inch pots (0.00226 sq meter) 2-3 weeks post germination, and ready for infiltration at approximately 6 weeks. Greenhouse lights set on a 16/8 h day/-night cycle. Temperature range: 23-28° C. Plant growth period was December-January 2019-2020. Geolocation: 43.52734733947189, 80.2289432466704.

### Agroinfiltration, Post-Infiltration Plant Growth and Plant Tissue Collection

After subculturing in shake flasks or bioreactors as described above, A. tumefaciens cultures were diluted to an OD₆₀₀ₙₘ=0.2 in agroinfiltration medium (10 mmol/L 2-N-morpholinoethanesulfonic acid, 10 mmol/L magnesium sulphate, pH 5.6; Garabagi et al., 2012b). Whole plant shoots (eight plant biological replicates per condition) were placed upside down in agroinfiltration medium inside a custom-made vacuum chamber and subjected to 90 kPa before releasing the vacuum. Infiltrated N. benthamiana were returned to the same greenhouse conditions for 1-7 days before sample collection. Whole infiltrated leaves were collected from plants at 0 day post infiltration (dpi), 1-, 3- and 7-days post infiltration (dpi), flash frozen in liquid nitrogen and stored at −80° C. until needed for further processing.

### Proteomics Sample Preparation

Protein extractions were performed as previously described (Prudhomme et al., 2022). Briefly, frozen leaf tissue (30 mg) was suspended in 100 mM Tris-HCl (pH 8.5) containing one protease inhibitor cocktail tablet (Roche, Basel, Switzerland). Leaf samples were homogenized using a Bullet Blender® Storm on power eight for 2 min with stainless steel beads (0.9-2 mm). Sodium dodecyl sulphate (2% final) and dithiothreitol (10 mM final) were added to the homogenized leaf samples, followed by incubation at 95° C. for 10 min with shaking at 800 rpm, cooling and the addition of iodoacetamide (55 mM final) was performed followed by room temperature incubation in the dark for 20 min. Next, ice-cold acetone (80% final) was added prior to storage at 20° C. overnight and samples were collected by centrifugation at 10 000 g at 4° C. for 10 min, washed twice with acetone (80%) and air-dried. Pellets were resolubilized in 8Murea/40 mM HEPES, diluted in ammonium bicarbonate (50 mM) and digested overnight with a mixture of LysC and trypsin proteases (Promega, Madison, WI, USA) (protein: enzyme ratio, 50:1). Digestion was stopped with 10% (v/v) trifluoroacetic acid, and 50 Ig of the acidified peptides was loaded onto STAGE- tips (STop And Go Extraction tips) (Rappsilber et al., 2007). Samples were dried in a SpeedVac at 45° C. for 45 min and stored at-20° C. until labelling was performed.

### Tandem Mass Tag (TMT) Labelling

Dried peptides were resuspended in 100 μL of 100 mM HEPES pH 8.5 and quantified by spectrophotometer at absorbance of 280 nm. An equivalent amount of peptide from all samples (i.e. 1.5 μg) was combined to create a common channel included to normalize across multiplex experiments. For each sample, 20 μg of peptides were labelled with an individual label from the TMTpro™ 16-plex isobaric label kit in a 10:1 label to peptide ratio. The individual samples were combined with a control/common sample to form the 16-channel multiplex. Excess label was quenched with 5 μL of 5% hydroxylamine. Multiplexed samples were cleaned with peptide desalting spin columns (Pierce, Dallas, TX, USA).

### Mass Spectrometry for Proteomics Samples

Samples were resuspended in 12 μL of buffer A (0.1% trifluoroacetic acid) with 6 μL of each sample analysed by nanoflow liquid chromatography on an Ultimate 3000 LC system (ThermoFisher Scientific, Waltham, MA) online coupled to a Fusion Lumos Tribrid mass spectrometer (ThermoFisher Scientific) through a nanoelectrospray flex-ion source (ThermoFisher Scientific). Samples were loaded onto a 5-mm u-precolumn (Thermo-Fisher Scientific) with a 300-μm inner diameter filled with 5-μm C₁₈ PepMap100 beads. Peptides were separated on a 15-cm column with a 75-μm inner diameter with 2-μm reverse-phase silica beads and directly electrosprayed into the mass spectrometer using a linear elution gradient from 4% to 30% acetonitrile (ACN) in 0.1% formic acid over 240 min at a constant flow of 300 nL/min. The linear gradient was followed by a washout with up to 95% ACN.

The Fusion Lumos mass spectrometer was operated in data-dependent mode, switching automatically between one full scan and subsequent MS/MS scans of the most abundant peaks with a cycle time of 3 s. Full scan MS1s were acquired in the Orbitrap analyser with a resolution of 120 000, scan range of 550-1800 m/z. The maximum injection time was set to 50 ms with an automatic gain control target of 4e5. The fragment ion scan was done in the lonTrap using a Quadrupole isolation window of 0.5 m/z and higher energy collisional dissociation fragmentation energy of (35%) 30 eV, maximum ion injection time of 35 ms and an automatic gain control target set to 1e4. TMTpro precursor ions were separated in the Orbitrap using an isolation window of 0.7 and collision energy of 55%. Orbitrap resolution was set to 60 000, scan range 100-500 m/z, maximum injection time 118 ms and automatic gain control target set to 5e5.

### Bioinformatics for Proteomics Samples

For proteome data analysis, RAW files were analysed using MaxQuant software (version 1.6.17.0.) (Cox and Mann, 2008). The derived peak list was searched with the built-in Andromeda search engine (Cox et al., 2011) with the reference A. tumefaciens (8 Jan. 2021; 5344 sequences, https://www.uniprot.org) supplemented with vector-specific sequences (Prudhomme et al., 2021a) and the reference N. tabacum (26 May 2020; 73 604 sequences, https://www.uniprot.org). The parameters were as follows: strict trypsin specificity, allowing up to two missed cleavages, minimum peptide length was seven amino acids, carbamidomethylation of cysteine was a fixed modification, N-acetylation of proteins and oxidation of methionine were set as variable modifications. A minimum of two peptides was required for protein identification, and peptide spectral matches and protein identifications were filtered using a target-decoy approach at a false discovery rate (FDR) of 1%. “Match between runs” was enabled with a match time window of 0.7 min and an alignment time window of 20 min (Cox et al., 2014). Relative quantification of proteins used Reporter ion MS3 with normalization as weighted ratio to the reference channel with isobaric weight exponent set at 0.75 (Yu et al., 2020).

In-depth proteome analysis and visualization performed using Perseus (version 1.6.2.2) (Tyanova et al., 2016). Proteins aligned to the reverse database, contaminants and proteins only identified with modified peptides were eliminated. Reporter ion intensities were converted to a log scale (log₂), and only those proteins present in valid-value filter of six in eight replicates in at least one group were used for further statistical processing. Missing values were imputed from a normal distribution (downshift of 1.8 standard deviations and a width of 0.3 standard deviations). The data were normalized by median subtraction. A Student's t-test identified proteins with significant changes in abundance (P≤0.05) with multiple hypothesis testing correction using the Benjamini-Hochberg FDR (Benjamini and Hochberg, 1995) cut-off at 0.05, S₀=1. A principal component analysis (PCA) was performed; replicate reproducibility was derived from a Pearson correlation with hierarchical clustering by Euclidean distance. For one-dimensional (1D) annotation enrichment, a Student's t-test (permutation-based FDR=0.05; S₀=1) was performed (Cox and Mann, 2012). This analysis generates a numerical “score” value, which represents the direction in which the protein reporter ion normalized intensities within a given category tend to deviate from the overall distribution of all proteins.

### Metabolomics Sample Prep

Frozen leaf tissue (100 mg) was added to 2-mL Eppendorf LoBind plastic tubes and Advance SSB14B Stainless Steel Beads (0.8 g; 0.9-2 mm diameter) and 300 μL cold 50% ACN were added to the samples followed by homogenization with the Bullet Blender Strom at power eight for 2 min. Samples were slow ramp centrifuged at 10 000 g at 4° C. for 10 min and 200 μL organic extract was transferred to 2-mL glass high-performance liquid chromatography (HPLC) vials.

### Metabolomics Mass Spectrometry (MS1)

Samples were analysed using a Thermo Ultimate 3000 ultra-high pressure liquid chromatography (UPLC) system coupled with a Thermo LTQ Orbitrap XL high- resolution mass spectrometer (HRMS) (Thermo Fisher Scientific). Separation was achieved using a Phenomenex Kinetex 1.7 μm C₁₈ column (100 Å, 50×2.1 mm) with a SecurityGuard ULTRA C₁₈ guard cartridge running a gradient of H₂O+0.1% formic acid (solvent A) and ACN+0.1% formic acid (solvent B) at a flow rate of 0.35 mL/min. The gradient method started with 5% B held for 0.5 min, increased to 95% B over 4.5 min and was held at 95% B for 3.5 min before returning to 5% B over 1 min and left to equilibrate for 3 min before the next injection. The HRMS scanned a range of 100-2000 m/z at a resolution of 30 000 in ESI⁺ mode using the following parameters: sheath gas flow 40, auxiliary gas flow 5, sheath gas flow 2, spray voltage 4.2 kV, capillary temperature 320° C., capillary voltage 35 V, tube lens 100 V, AGC target 5E5 and maximum ion time 500 ms. Samples were injected in a randomized order with a solvent blank injected every 10 samples to assess for metabolite carryover. A reserpine standard was injected at the beginning of every sample sequence to confirm method accuracy and instrument calibration and to aid downstream data processing.

### Metabolomics MS1 Data Processing

UPLC-HRMS profiles (.RAW files) from organic samples, the nine method blanks and 11 ACN blanks, were uploaded into Mzmine version 2.53 (Pluskal et al., 2010). Mass detection was performed using the exact mass function to generate a list of ions in each scan with a minimum intensity level of 1.0e4. The Automated Data Analysis Pipeline (ADAP) chromatogram builder was used to build chromatograms for each mass that detected continuously over scans using the following parameters: Minimum group #in size of scans=5, group intensity threshold=1e5, minimum highest intensity=5e5, m/z tolerance=0.005. Chromatogram deconvolution was used to separate individual peaks in the constructed chromatograms using a local minimum search: chromatographic threshold=20%, search minimum in retention time (RT) range (min)=0.05, minimum relative height=10%, minimum ratio of peak top/edge=20, peak duration range (min)=0.00-10.00. Isotopic peak grouping was used to remove additional isotopic peaks generated for the same mass feature leaving only the most intense peak: m/z tolerance=0.005, retention time tolerance=0.1, monotonic shape=yes, maximum charge=1, representative isotope=most intense, remove original peak list=no. To achieve accurate comparisons between .RAW files, the Join Aligner was used align shifts in m/z intensity and RT across samples: m/z tolerance=0.005, weight for m/z=20, RT tolerance=0.1, weight for RT=10, defaults settings were used for other parameters. To reduce missing values for features detected in some samples but below the noise level in others, gap filling was performed using the same RT and m/z range to check for the presence of designated mass features occurring below original pre-processing thresholding values: m/z tolerance=0.001. Peak area data were then exported from MZmine to a .csv file for manual curation (mass features that were found in two or more blanks and/or in less than 70% of replicates within a sample group were removed). The curated .csv file was converted into a tab delimited .txt file and uploaded into Perseus version 1.6.2.2 (Tyanova et al., 2016). Data were log2 transformed to scale and aid in measuring fold changes between samples; missing values were replaced from the normal distribution in each sample using a Gaussian distribution width relative to the standard deviation of measured values of 0.3 and a down shift (the amount by which the distribution used for the random numbers is shifted downward in units of the standard deviation of measured values) of 1.8. Finally, the data were normalized using subtraction of the median. A Student's t-test identified mass features with significant changes in abundance (P≤0.05) with multiple hypothesis testing correction using the Benjamini-Hochberg FDR cut-off at 0.05, S₀=1.

### Metabolomics MS/MS

To annotate mass features designated as significant from our metabolomics analysis, four samples with the highest relative intensities for significant features were selected for MS2 analysis using a Proxeon EASY nLC II System (Thermo Fisher Scientific) coupled with a Q-Exactive Plus mass spectrometer (Thermo Fisher Scientific). Metabolite separation was achieved on an Acclaim PepMap RSLC C₁₈3 μm column (100 A, 75 lm 9 15 cm) running a gradient of H2O+0.1% formic acid (solvent A) and ACN+0.1% formic acid (solvent B) at 230 nL/min. The column was held at 10% B for 10 min, increased to 100% B over 35 min, held for 10 min before returning to starting conditions. Full MS1 scans were acquired in ESI+ mode with a resolution of 70 000, scan range of 100-1500 m/z, maximum injection time 100 ms and AGC target 3e6. The data-dependent MS2 scan was acquired using an isolation window of 2 m/z, resolution of 17 500, scan range of 200-2000 m/z, maximum ion injection time of 100 ms and an AGC target of 1e5.

### Metabolomics MS/MS Data Processing

For MS2 metabolome data analysis, .RAW files were analysed using MS Dial software (Tsugawa et al., 2015) and Thermo Scientific Compound Discoverer 3.3.1.111 (CD). Parameters for MS Dial are as follows: Mass accuracy tolerance MS1=0.01, MS2=0.025, minimum peak height=1e6, mass slice=0.1 Da, and deconvolution with a sigma window value=0.5. Mass feature identification was done using the MSMS-Public-Pos-VS15 database with identification score cut off of 80%. Alignment was done using a retention time tolerance of 0.05 min and MS1 tolerance of 0.015 Da. Data processing in CD involved several nodes. In the Select Spectra node under Peak Filters, S/N Threshold (FT-only)=1.5 and under General Settings, Precursor Selection=Use MS (n−1) Precursor and Use Isotope Pattern in Precursor Reevaluation=True. In the Detect Compounds node under General Settings, Mass Tolerance [ppm]=5 ppm, Min. Peak Intensity=5000, Min. #Scans per Peak=5, Use Most Intense Isotope Only=True and under Peak Detection, Chromatographic S/N Threshold=1.5. In the Group Compounds node under General Settings, Mass Tolerance=5 ppm, RT Tolerance [min]=0.2 and Area Integration=Most Common lon. In the Assign Compound Annotations node two data sources were used, mzCloud Search and ChemSpider Search (CSID), under Scoring Rules, Use mzLogic=True. In the Search mzCloud node under General Settings, Compound Classes=Endogenous Metabolites, Natural Products/Medicines, Natural Toxins, Precursor Mass Tolerance=10 ppm, FT Fragment Mass Tolerance=10 ppm and IT Fragment Mass Tolerance=0.4 Da.

Overlap between MS Dial and CD was four metabolites with identical annotations (12-Oxo phytodienoic acid, adenine, riboflavin and feruloyltyramine), four metabolites with agreement on formula or type of compound with MS Dial having better confidence (feruloyl putrescine, feruloyl O-methyldopamine, 2(1H)-Naphthalenone, 4a,56788ahexahydro-6-hydroxy-38-dimethyl-5-(1-methylethyl)—, (4aR,5S,6S,8R,8aS)— and 2-Cyclohexen-1-one, 4-[(1E)-3-(beta-Dglucopyranosyloxy)-1-buten-1-yl]-4-hydroxy-355-trimethyl—, (4S)—), one metabolite with agreement on formula or type of compound with CD having better confidence (7-hydroxycoumarine), and five metabolites specific to MS Dial (phenylacetaldehyde, 5-p-coumaroylquinic acid, myrcene, Naphtho[23-b]furan-2(4H)-one, 4a, 56788a,99a-octahydro-9ahydroxy-34a,5-trimethyl—, (4aR,5S,8aS,9aR)— and 2-Butanone, 4-[3-(beta-D-glucopyranosyloxy)-4-hydroxy-266-trimethyl-1-cyclohexen-1-yl]—). Annotations were matched to MS1 data using a m/z tolerance of 5 ppm. Annotated metabolites and proteins were matched to appropriate metabolic pathways using the Kyoto Encyclopedia of Genes and Genomes (KEGG) (Kanehisa and Sato, 2019; Ogata et al., 1999). Proteins with significant differences in abundances corresponding to changes in metabolite abundance were mapped to same metabolic pathways to integrate the proteome and metabolite data.

### Western Blot

Aliquots of undigested protein extract from the proteomic sample preparation were concentrated, separated by 10% SDS-PAGE and stained with Coomassie blue or transferred to a polyvinylidene fluoride (PVDF) membrane using the Trans-bot Turbo transfer apparatus according to the manufacturer's instructions (Bio-Rad, Hercules, CA, USA). For the trastuzumab western blot, membranes were blocked overnight in 4% non-fat skim milk in 10 mL Tris-buffered saline (TBS) (50 mM Tris, 150 mM NaCI, pH 7.5) at 4° C. Blocked membranes were probed using alkaline phosphatase-conjugated goat anti-human IgG (c-specific), and alkaline phosphatase-conjugated goat anti-human kappa light chain antibody for 1 h at room temperature (approx. 21° C.) with an antibody concentration of 1:5000 diluted in the same blocking solution. Membranes were washed five times with TBSTween (19 TBS, 0.05% Tween-20) and developed using SIGMAFAST™ BCIP®/NBT tablet dissolved in water as per manufacturer's instructions (Sigma-Aldrich, St. Louis, MO, USA). Imaging was performed with Bio Rad Universal Hood III and Image Lab 6.0.

shRNA Design

Post-transcriptional gene silencing was used to determine whether the proteases of interest are associated with degradation of trastuzumab in planta. Short hairpin RNAs (shRNAs) were designed complementary to the three target aspartic proteases: aspartic proteinase A1-like; LOC107809122 (AP A1), aspartyl protease AED3-like; LOC107806163 (AED3-1), aspartyl protease AED3-like; LOC107828826 (AED3-2). Genome sequences were confirmed with SolGenomics (https://soIgenomics.net/tools/blast/) and CDNA sequences were used for shRNA design with VectorBuilder (https://en.vectorbuilder.com/tool/shrna-targetdesign.html) and GPP Web Portal (https://portals.broadinstitute.org/gpp/public/seq/search), for suppression of the three aspartic proteases of interest and a common shRNA was also designed for the AED3-1/2-like proteases. Sequences were annotated to add a hairpin loop and to reverse complementary sequence to form the shRNA structure. Restriction sites for HindIII and SacI were added for cloning.

### Cloning and Agroinfiltration

The shRNA coding sequences were synthesized and cloned into p1407 (PlantForm proprietary plasmid). Plasmids were transformed by heat-shock into competent Escherichia coli cells and spread on agar plates with kanamycin (50 mg/mL). Colonies were selected and cultured in LB at 30° C. overnight, plasmids were purified and verified by restriction digestion with SphI and sequencing. Transformation into A. tumefaciens cells was performed via electroporation and confirmed with restriction enzyme digestion (Sph1) followed by shake flask culturing and agroinfiltration as outlined above. Each group of N. benthamiana plants (10 biological replicates) were co-infiltrated with the pPFC0058 and shRNA except the control group, which was only infiltrated with pPFC0058. Plants were grown in a greenhouse (conditions outlined above) for 7 dpi followed by harvesting of the leaves and storage at −80° C. until testing.

### Processing and Trastuzumab Detection

At 7 dpi, 10 g of leaf sample was blended with a Polytron in 30 mL 1xPBS for 1 min with 1 mL transferred to an Eppendorf tube and centrifuged at 10 000 g for 20 min at 4° C. Samples were kept on ice to maintain integrity while testing. The concentration of trastuzumab in each sample was measured using the commercial Blitz™ system (Fortebio, Fremont, CA, USA). This system uses biolayer interferometry to detect the concentration of target protein (i.e. trastuzumab) in each sample and is the industry standard for quantification of the target protein. The amount was calculated in mg/kg and measured in 10 biological replicates and three technical replicates. For validation of trastuzumab production within the samples prepared for proteomics, trastuzumab was measured using the Blitz™ system at 1, 3 and 7 dpi. Trastuzumab values were normalized to protein concentrations for each replicate.

### Statistical Analysis

To measure differences in trastuzumab production, a Welch's t-test (assuming unequal variances and a normal distribution of the data) was performed between the control and single shRNA treatments (i.e. AED3-1, AED3-2, AP A1). A Kruskal-Wallis test (non-parametric test) was performed between the control and double shRNA treatments (i.e. AED3-1/2). P-values are reported.

### Results

### Global Plant Proteome Remodelling Is Defined by Temporal and Bacterial Growth Axes

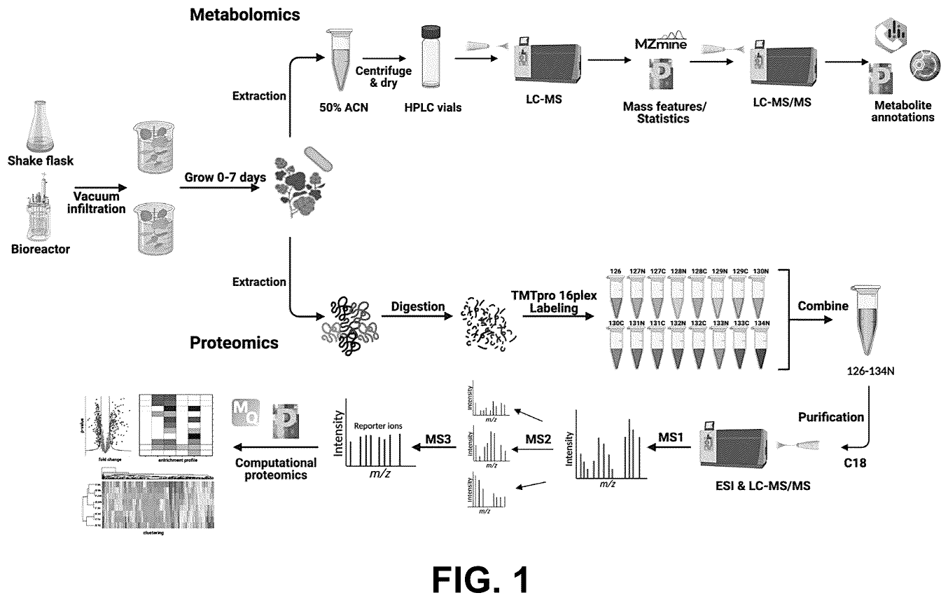

To examine the role of A. tumefaciens growth conditions on the production of the trastuzumab heavy and light chains (i.e. target protein), and assess changes in host defence responses, a temporal quantitative proteomics and metabolomics analysis of agroinfiltrated N. benthamiana was performed (FIG. 1). The objective of this study was to identify specific proteins and metabolites that may limit the accumulation of the target protein along with how the interaction between the plant and pathogen systems fluctuate throughout the molecular pharming process. A global proteome assessment identified 2542 proteins from N. benthamiana and 343 proteins from A. tumefaciens (FIG. 2A). A principal component analysis (PCA) defined component 1 based on the time post infiltration (35.1% explained variance) and component 2 based on bioreactor or shake flask A. tumefaciens growth prior to agroinfiltration (13.4% explained variance) (FIG. 2B; FIG. 9). A greater separation between shake flask and bioreactor growth at the early sample collection time points (0 dpi, 1 dpi, 3 dpi) compared to the later (7 dpi) time point was observed, demonstrating temporal differences in proteome remodelling driven by the bacterial growth conditions. Visualization of protein intensities by hierarchical clustering based on Euclidian distance further illustrated the influence of time and bacterial culture conditions on the proteomes (FIG. 2C).

### Bacterial Growth Conditions Modulate Production of Trastuzumab at Early Time Points

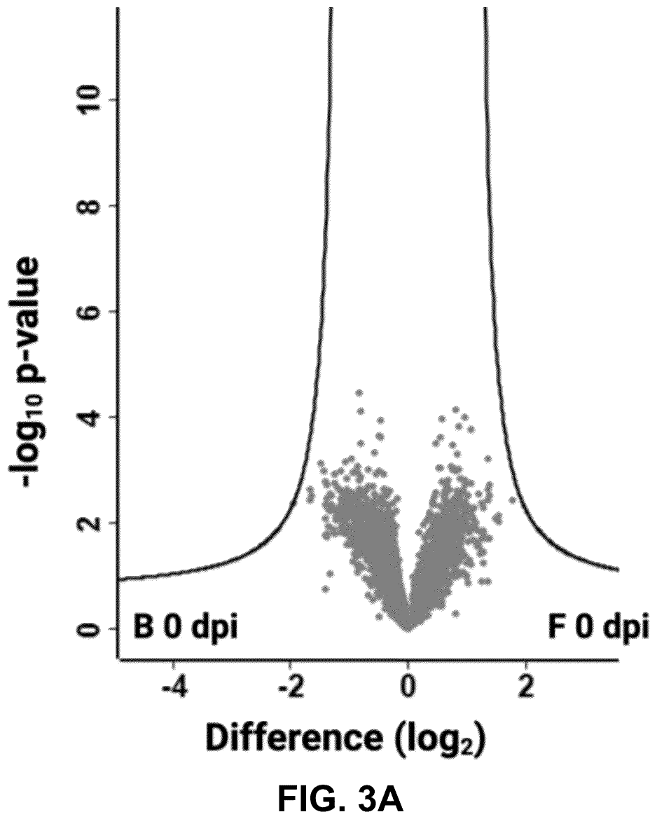

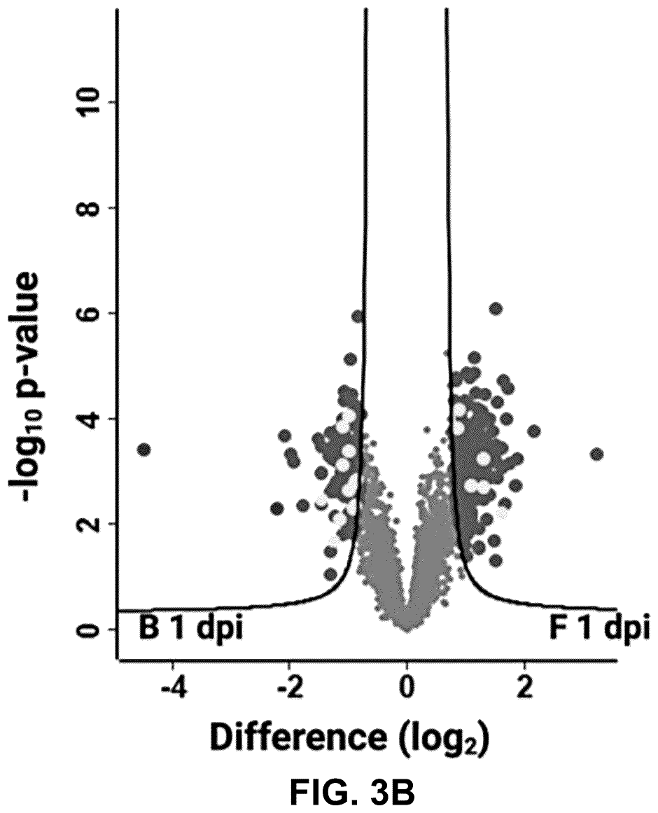

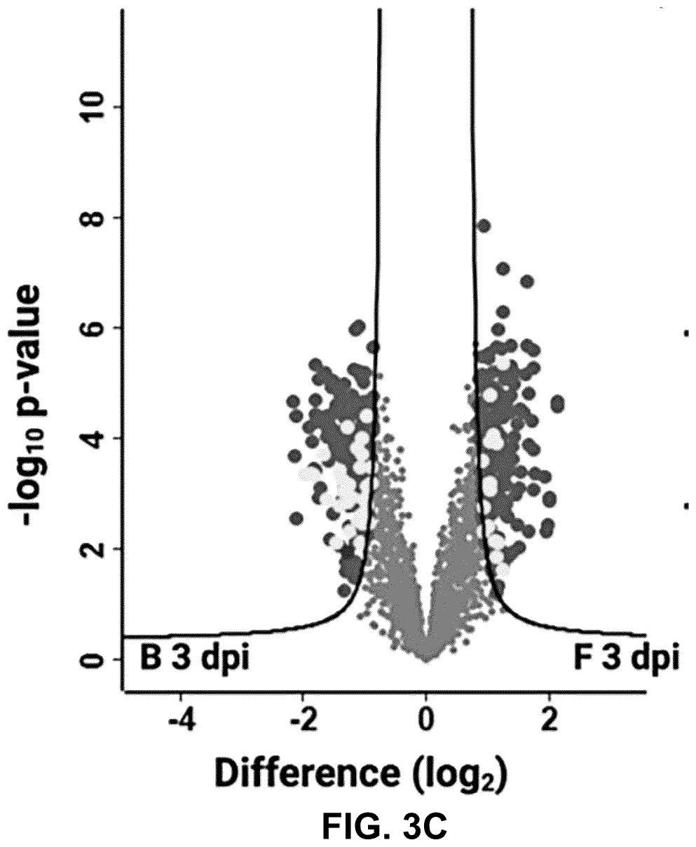

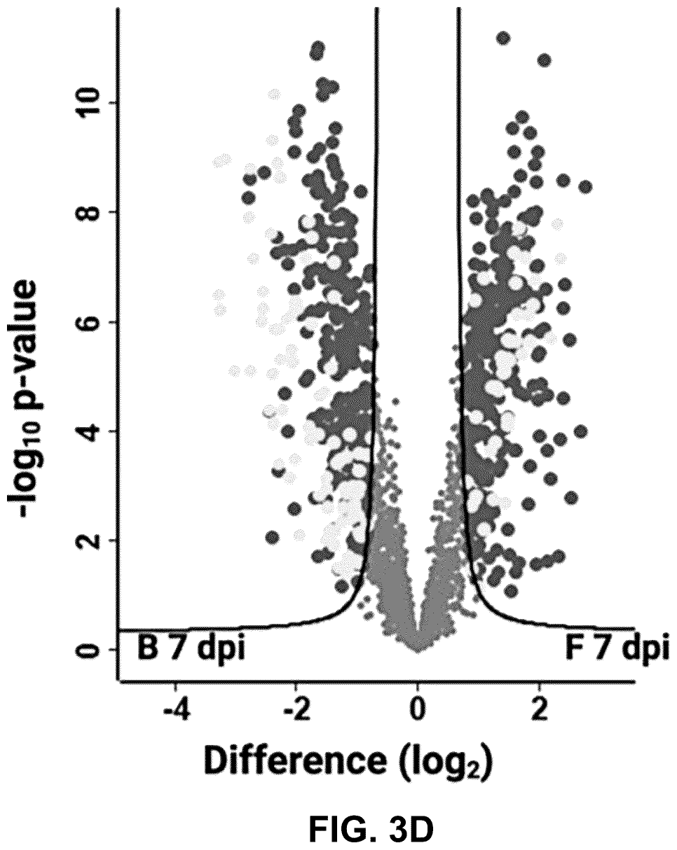

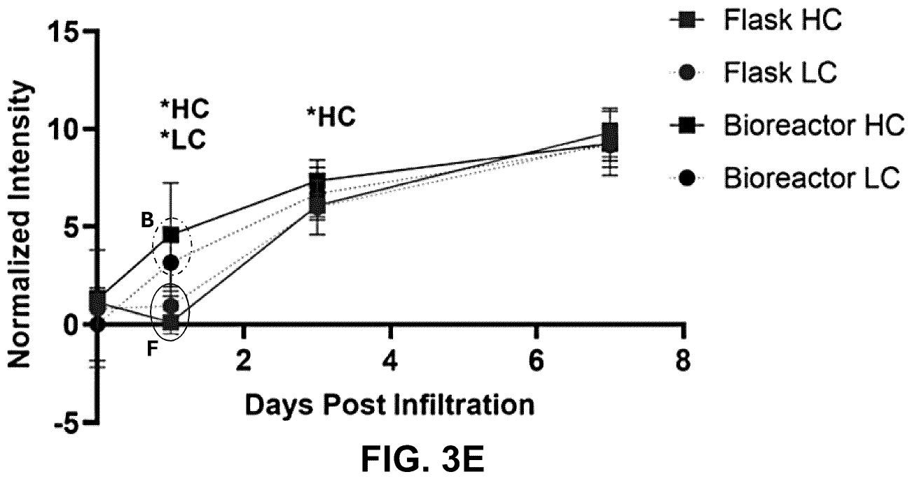

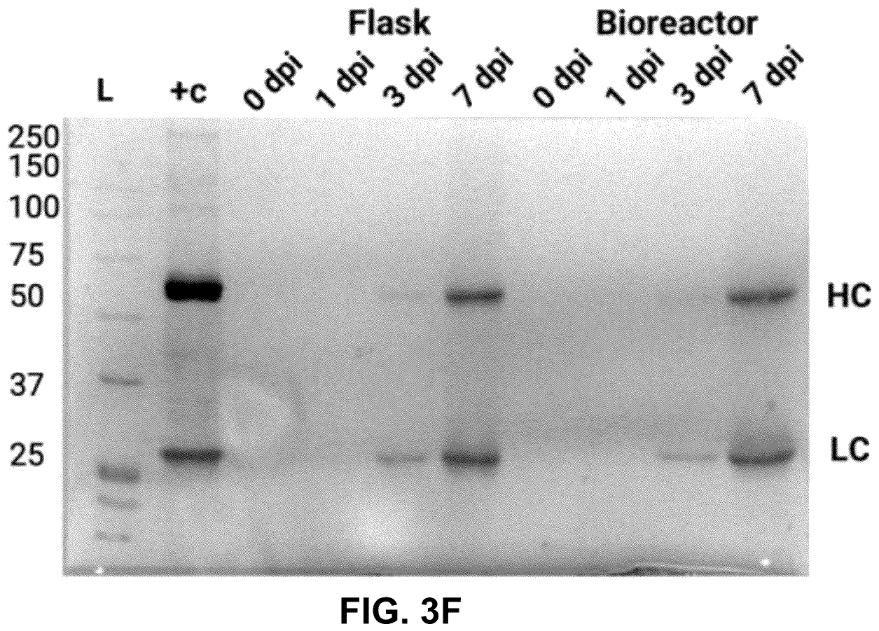

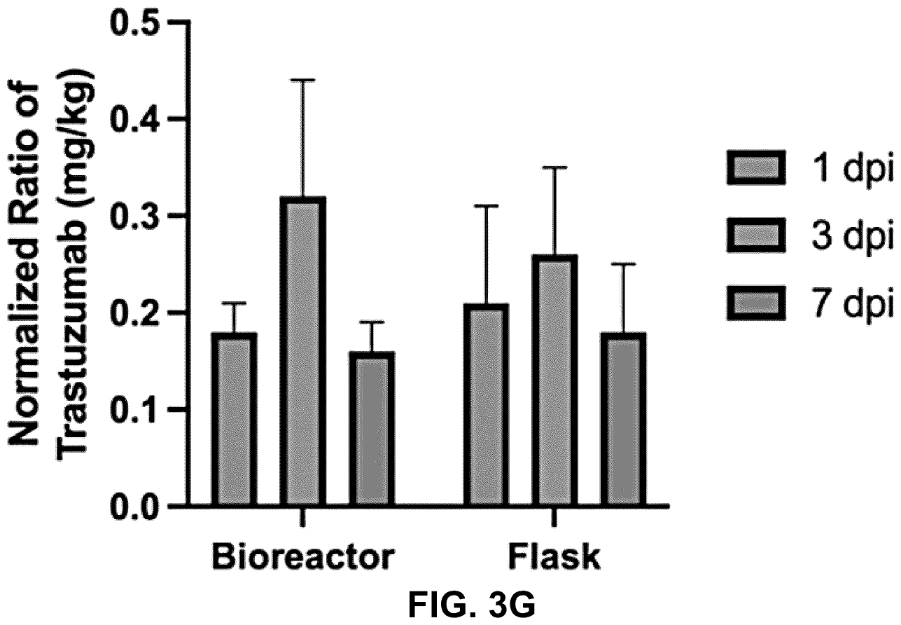

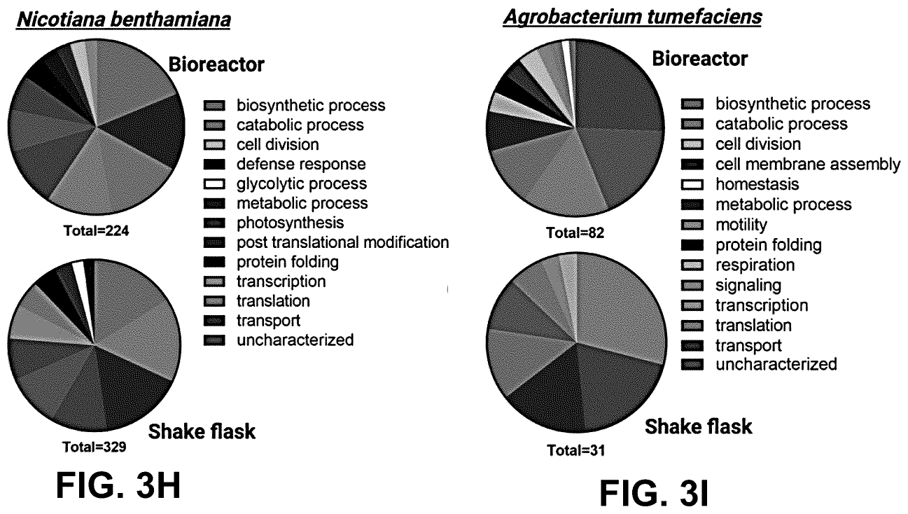

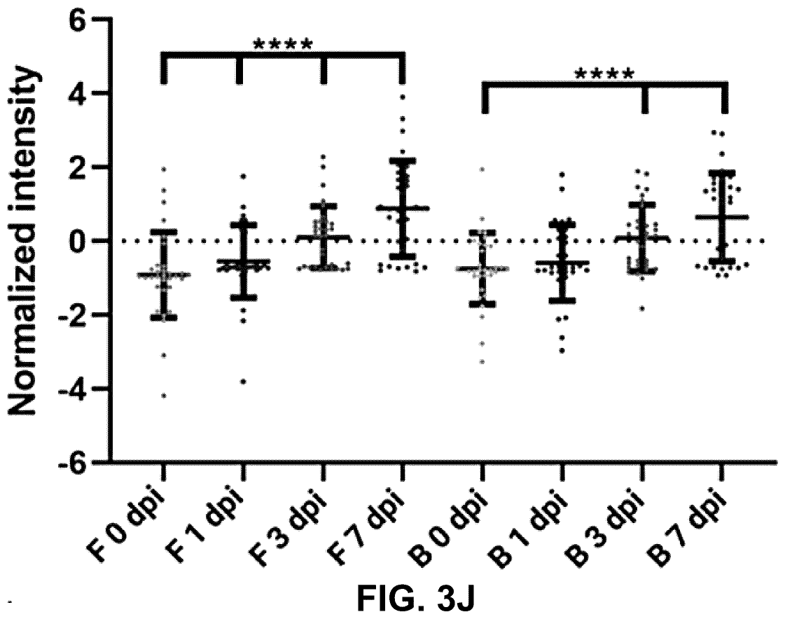

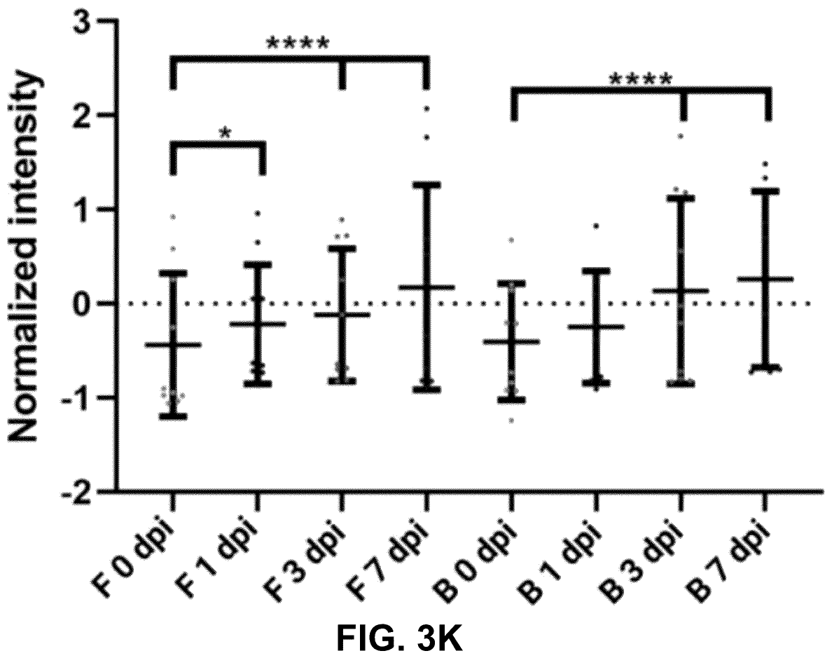

It was previously showed that distinct growth conditions for A. tumefaciens, such as shake flask versus bioreactor, supported priming of the bacterium for plant infection following bioreactor growth (Prudhomme et al., 2021a). For plant infiltrations, bacteria were collected at OD₆₀₀ₙₘ of 1.4-1.5 following bioreactor or shake flask growth conditions, which corresponded to a collection time of 12 h versus 18 h, respectively. Colony-forming unit (CFU) counts at this collection time point quantified 1.05×10¹²±5.23×10¹¹ CFU/mL for shake flask growth conditions and 2.17×10⁹±9.4×10⁸ CFU/mL for bioreactor growth conditions. In the present study, the impact of bacterial growth conditions over a time course of N. benthamiana infection was explored. At 0 dpi, no significant difference was observed in protein production by the bacterium or the plant regardless of the bacterial culturing conditions (FIG. 3A). However, at 1 dpi, 150 proteins (136 plant,14 bacterial) were significantly more abundant following bacterial bioreactor growth compared to bacterial shake flask growth with 241 significantly different proteins (235 plant, six bacterial) (FIG. 3B). At 3 dpi, 245 proteins (210 plant, 35 bacterial) were significantly more abundant following bacterial bioreactor growth, and 226 proteins (210 plant, 16 bacterial) were significantly more abundant following shake flask bacterial growth (FIG. 3C). At 7 dpi, 342 proteins (257 plant, 85 bacterial) were significantly higher in abundance following bacterial bioreactor growth and 351 proteins (310 plant, 41 bacterial) were significantly more abundant following bacterial shake flask growth (FIG. 3D).

Next, the production of the trastuzumab heavy and light chains over time and between bacterial growth conditions was monitored. There was a significant increase in the production of the heavy and light chains at 1 dpi following bacterial bioreactor growth, a consistent increase in the heavy chain at 3 dpi, however, by 7 dpi, there was no significant differences in trastuzumab production regardless of the bacterial growth condition (FIG. 3E). The inventors confirmed trastuzumab production at 3 and 7 dpi via western blot for both agroinfiltration methods (FIG. 3F; FIGS. 10A, 10B) and further validated the increase in trastuzumab production at 3 dpi following bioreactor infiltration using the Blitz system (the industry-standard method for quantifying target protein production; Langley et al., 2022; Lua et al., 2015; FIG. 3G).

Distribution of these significantly different proteins were defined by Gene Ontology Biological Processes (GOBP) (Ashburner et al., 2000). For N. benthamiana, the number of proteins associated with biosynthetic processes, metabolic processes, transport, uncharacterized, photosynthesis, protein folding, and post-translational modifications were relatively stable regardless of bacterial growth condition (FIG. 3H). However, differences were noticed in the number of proteins associated with catabolic processes, translation, transcription, glycolytic processes and defence responses, between the bacterial growth conditions. For A. tumefaciens, the number of proteins associated with transport, biosynthetic processes, and respiration were relatively stable between shake flask versus bioreactor growth conditions categories (FIG. 31). Conversely, proteins associated with motility, signalling and protein folding were observed in higher numbers following bioreactor growth. These findings align with the previous observations of bacterial priming driven by culture conditions (Prudhomme et al., 2021a).

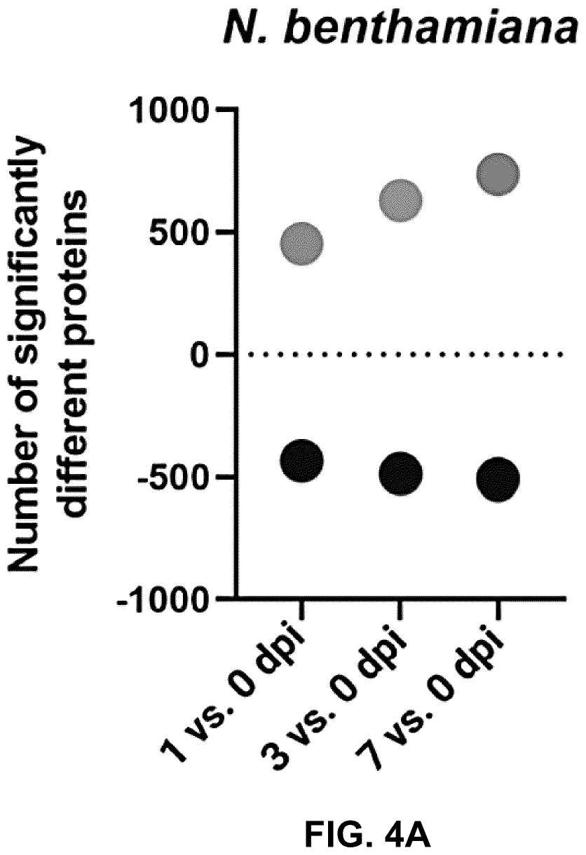

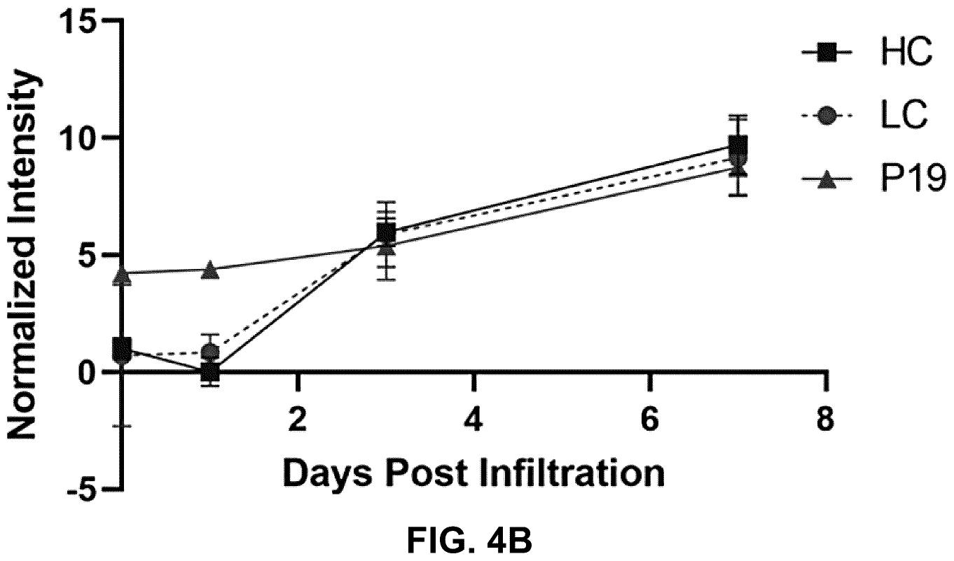

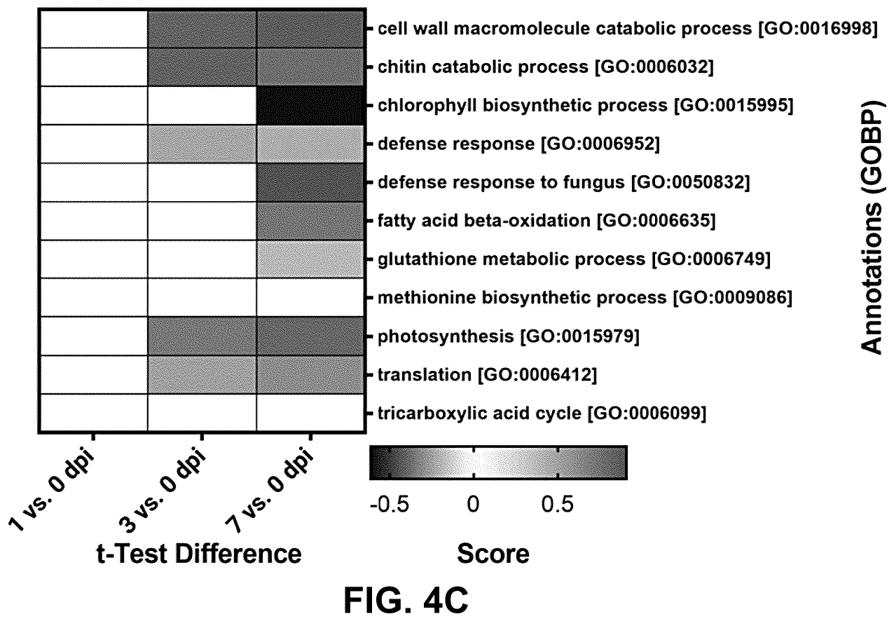

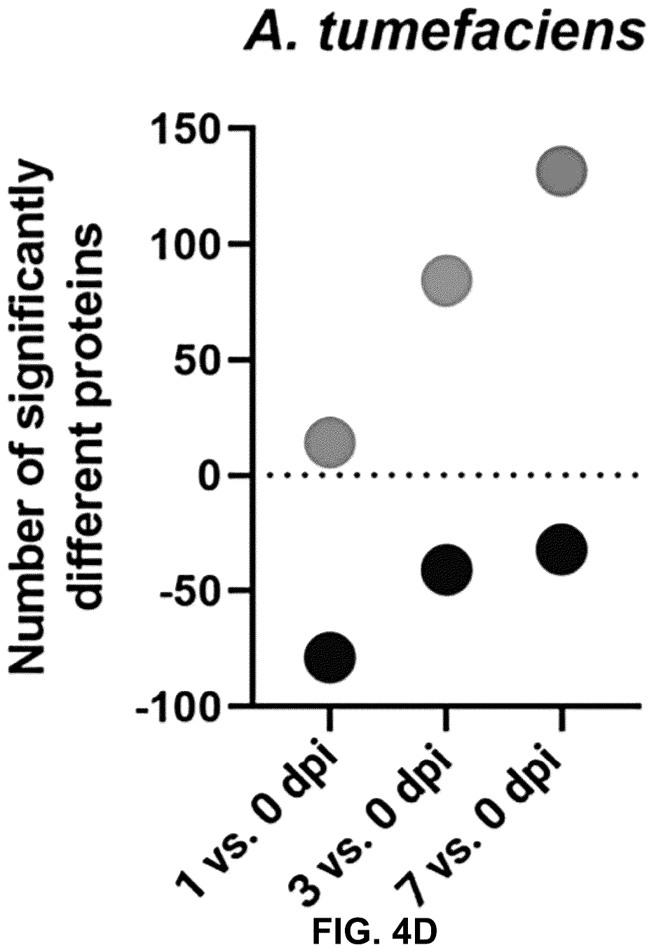

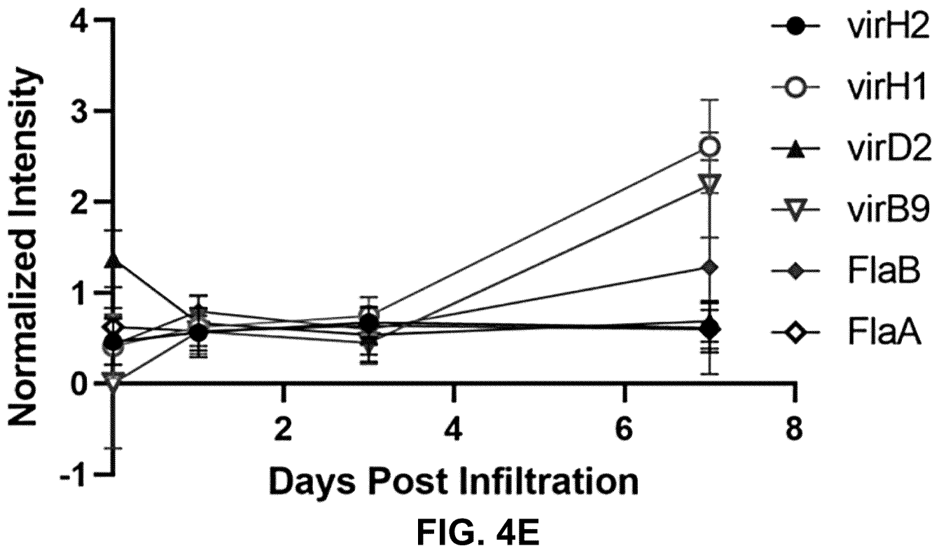

Given the observations and the defined relationship between A. tumefaciens virulence and N. benthamiana defence response (Grosse-Holz et al., 2018a), production of defence-associated plant proteins across time and bacterial culturing conditions was monitored (FIG. 3J; Table 1). A significant and consistent increase in production of plant defence proteins following agroinfiltration with shake flask-grown bacteria was observed beginning at 1 dpi, whereas as significant increase in plant defence-associated proteins following agroinfiltration with bioreactor-grown bacteria was not detected until 3 dpi. Intriguingly, these delayed plant defence response corresponded with earlier production of the trastuzumab heavy and light chains in the bioreactor samples. These data suggest that the early plant defence response induced by shake flask-grown A. tumefaciens limit initial target protein production during molecular pharming practices. To further define the plant defence responses, the data for proteins with known interference properties during molecular pharming: proteases were analysed (Gross-Holz et al., 2018a, 2018b). A significant increase in apoplastic protease production was observed at all time points following bacterial shake flask growth compared to a delayed induction of plant proteases at 3 dpi following bacterial bioreactor growth (FIG. 3K; Table 2). Together, these data define temporal and bacterial growth specific regulators of protein production within the evaluated biological system and propose drivers of differential trastuzumab production. Given the observed differences in host and pathogen responses mediated by bacterial growth conditions and time, the impact of shake flask versus bioreactor growth was teased apart as an independent driver of trastuzumab production over time. Comparison across time following shake flask bacterial growth shows a steady increase in the number of significantly different N. benthamiana proteins over time (FIG. 4A). Monitoring of trastuzumab heavy and light chains, as well as P19 (RNA silencing suppressor) increased over time with initial antibody detection at 3 dpi and peak production at 7 dpi (FIG. 4B). Enrichment analysis by 1D annotation (Cox and Mann, 2012) based on GOBP for N. benthamiana proteins showed significant positive enrichment in cell wall catabolic process, defence response, fatty acid ß-oxidation, and glutathione metabolic process as time after agroinfiltration increased (FIG. 4C). Conversely, the enrichment of proteins associated with chlorophyll biosynthetic process, photosynthesis and translation at the initial time point was observed.

For A. tumefaciens, an increased number of significantly different proteins was observed as infection progressed (FIG. 4D). To assess bacterial virulence factor production over time, protein intensity was mapped with a focus on classical virulence factors, including VirB9 (outer-membrane-associated component of the Type 4 Secretion System [T4SS]), VirD2 (T-DNA transfer), VirH1 (P450-type monooxygenases) and VirH2, along with two flagellar subunit proteins (FlaA, FlaB) associated with cell motility. Production of these proteins was relatively low and stable until 3 dpi when VirH1 and VirB9 showed a significant increase, whereas abundance of VirD2, VirH2 and FlaA remained unchanged (FIG. 4E). Taken together, these data support temporal remodelling of the plant and pathogen following agroinfiltration with initial trastuzumab production at 3 dpi, aligning with bacterial virulence factor production.

### Bioreactor Bacterial Growth Conditions Influences Rapid and Sustained Trastuzumab Heavy and Light Chain Production and Earlier Classic Bacterial Virulence Factor Production

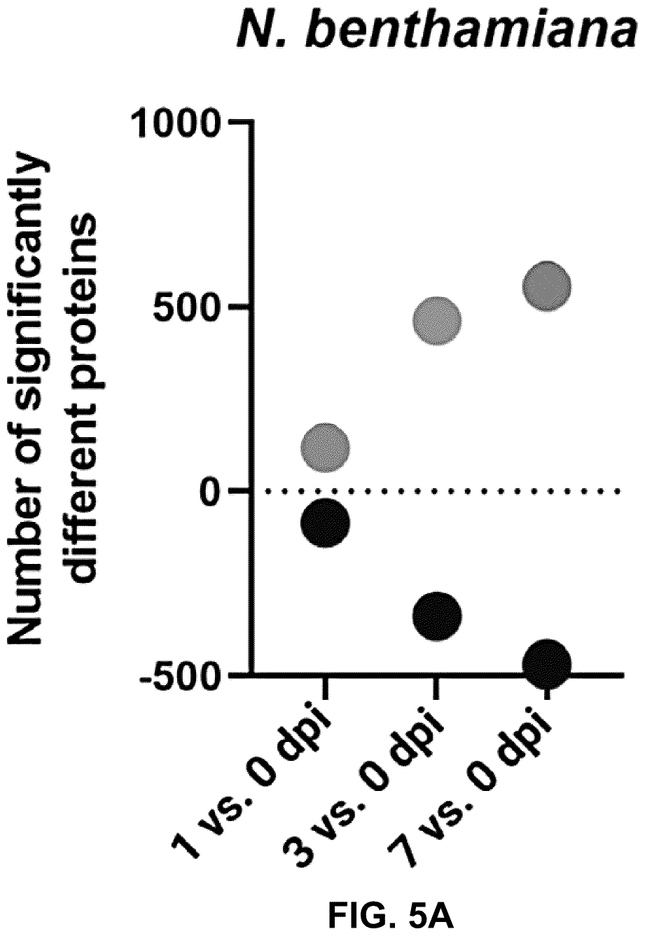

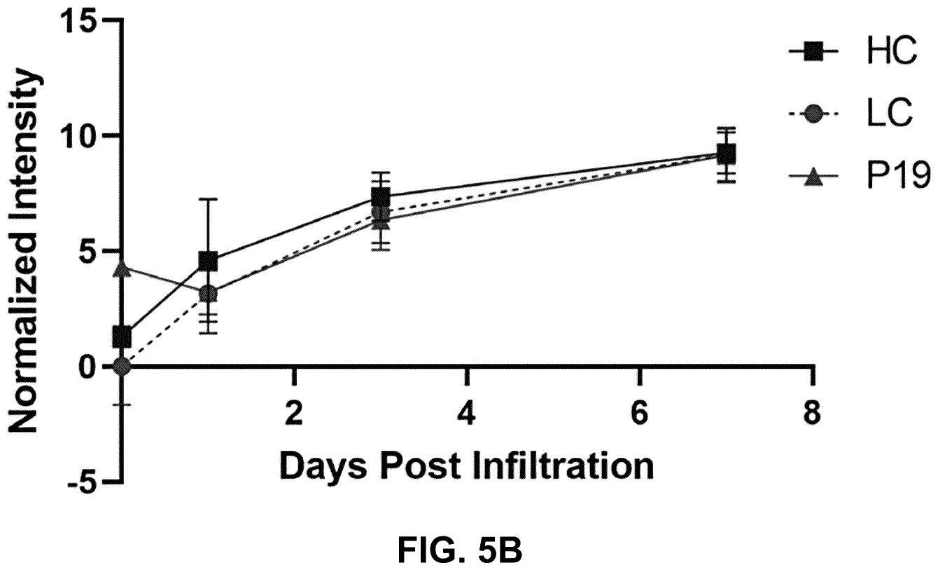

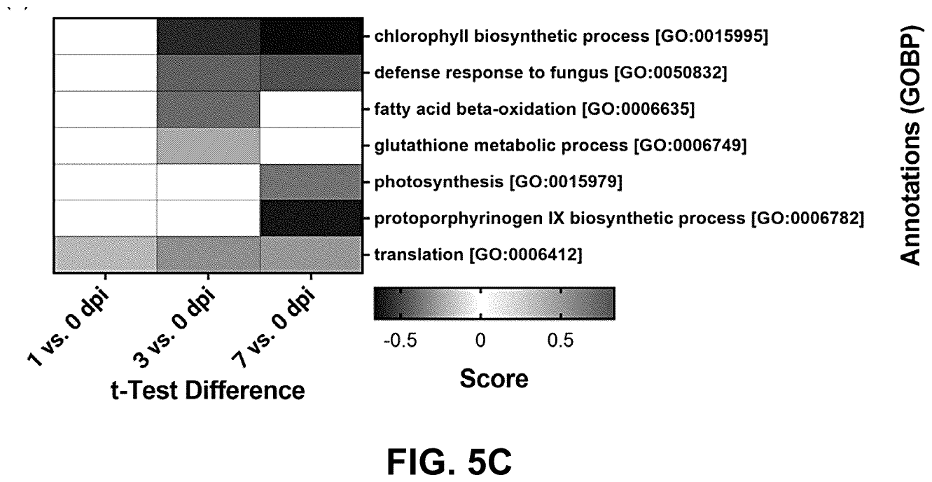

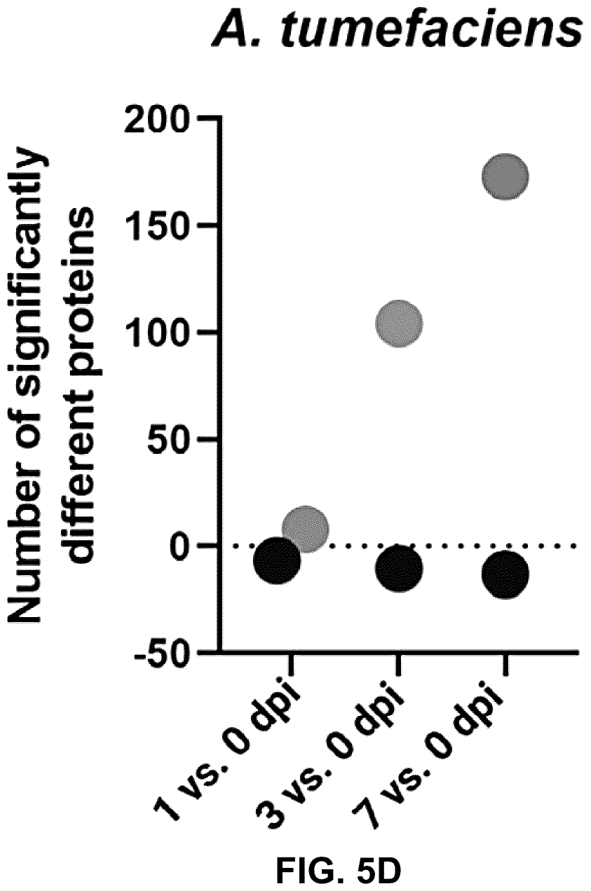

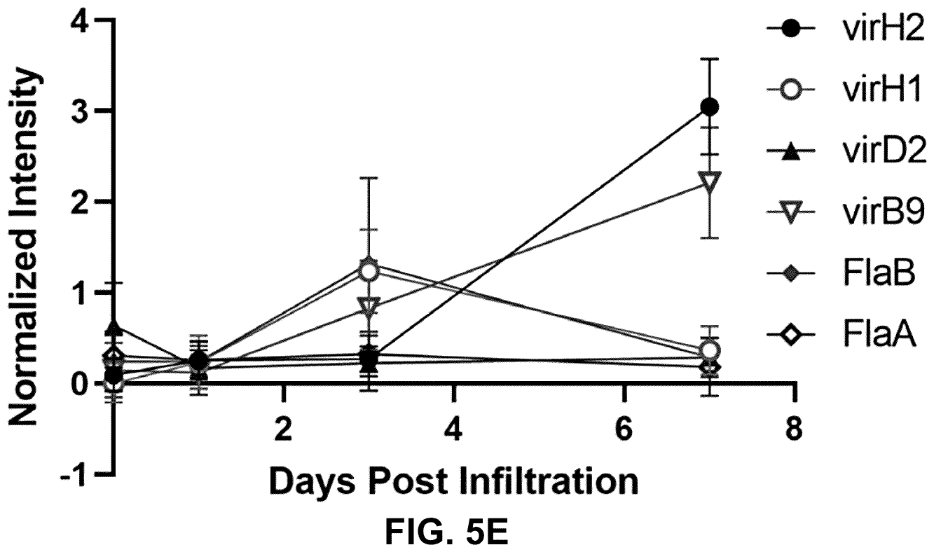

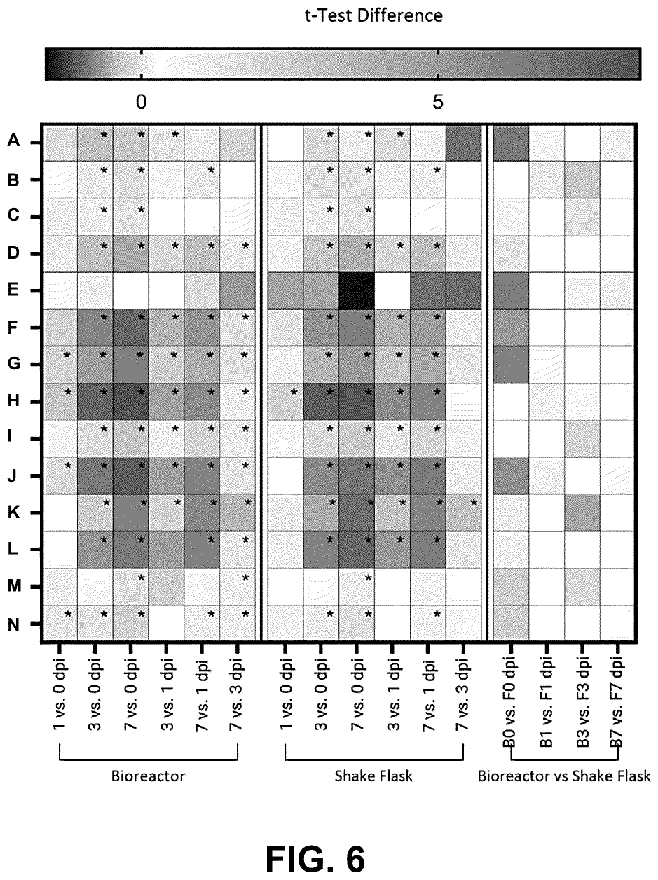

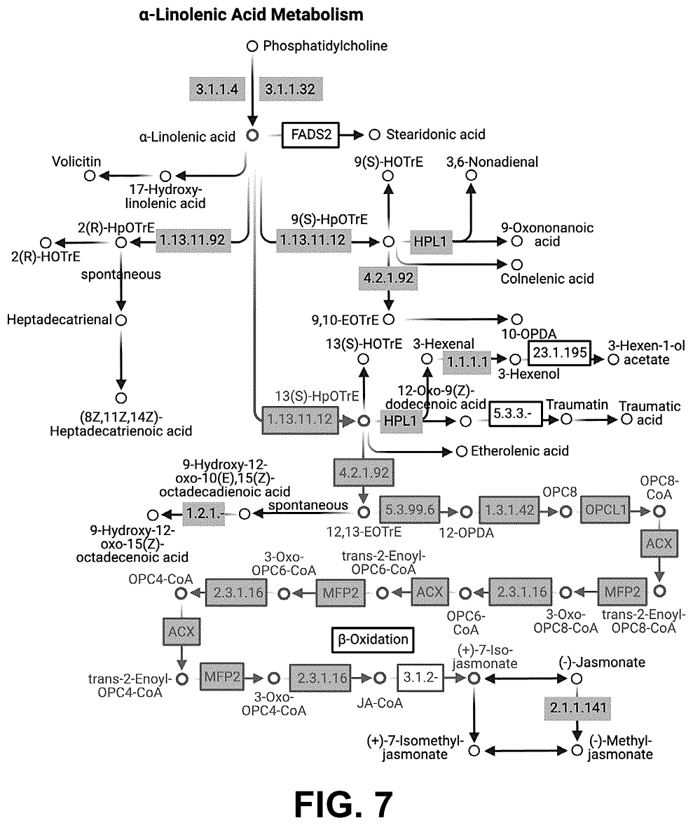

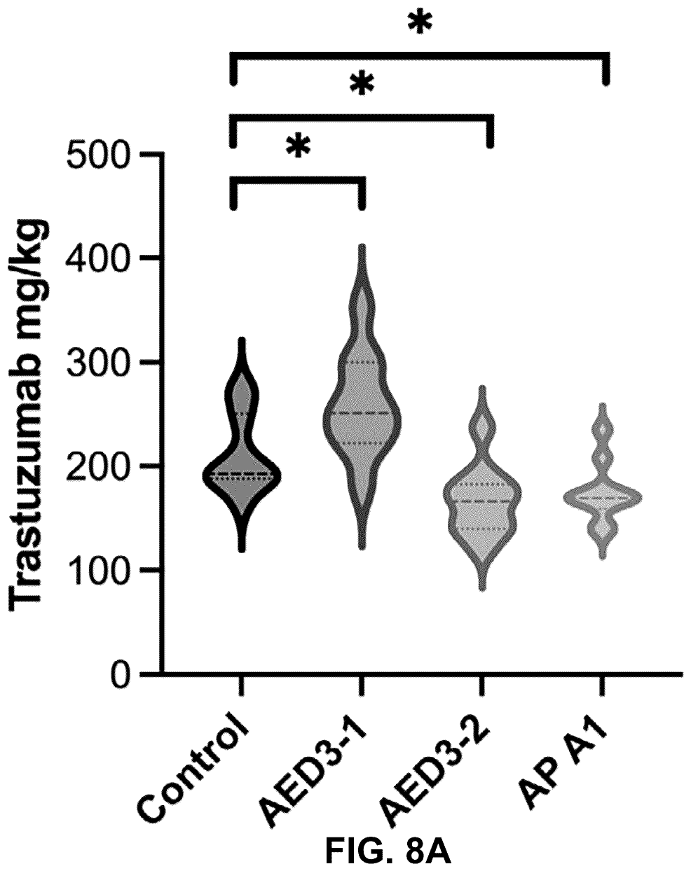

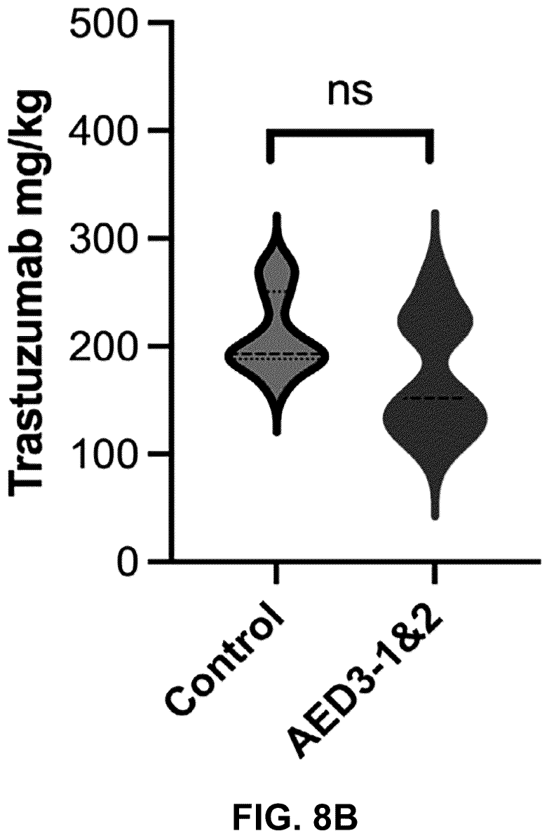

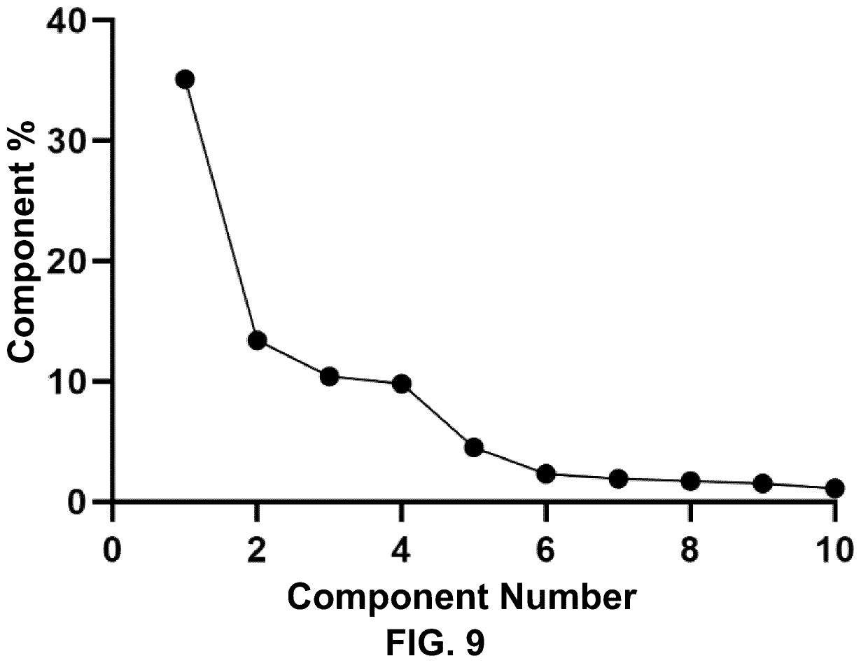

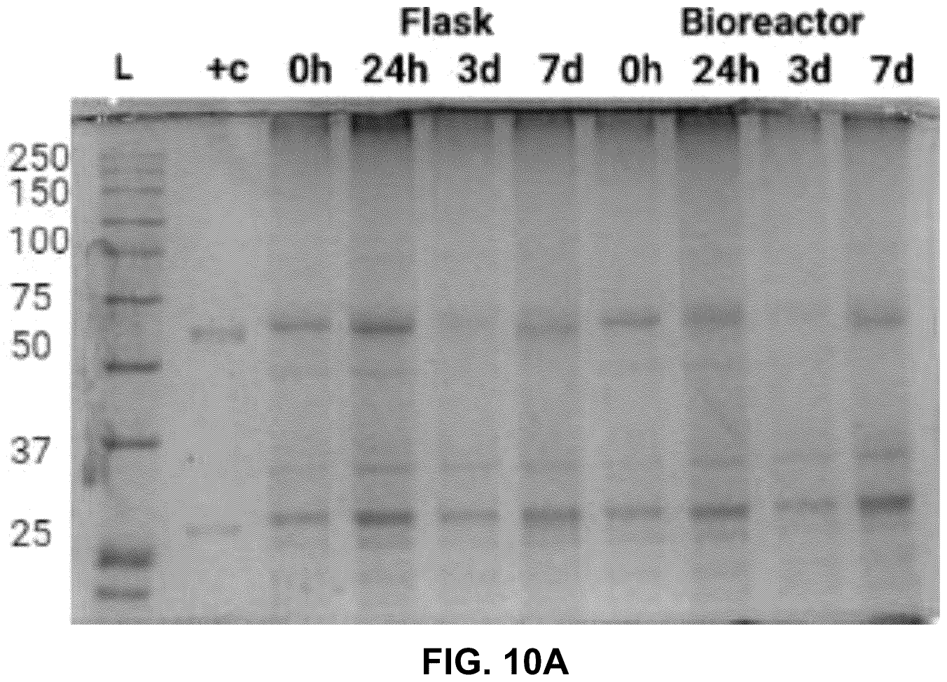

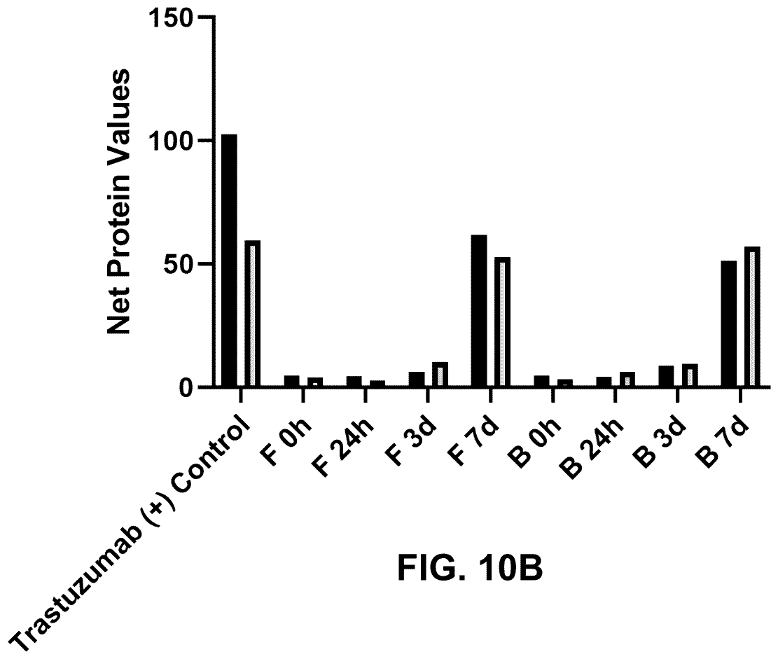

Based on the previous hypothesis of bacterial priming for motility and virulence (Prudhomme et al., 2021a) during bioreactor growth, these differences on trastuzumab production were evaluated over time. Comparison across time following bioreactor bacterial growth shows a steady increase in the number of significantly different N. benthamiana agroinfiltrated with bacteria from shake flask growth, indicating that global drivers of host response are associated with general processes (e.g. growth, adaptation). Monitoring of trastuzumab heavy and light chains, as well as P19 production increased over time with initial antibody detection at 1 dpi with a consistent increase to 7 dpi, demonstrating an earlier initiation of trastuzumab production following bioreactor bacterial growth compared to shake flask bacterial growth (FIG. 5B). Notably, final (7 dpi) trastuzumab production levels by heavy and light chain intensities did not significantly differ between the bacterial growth conditions. Enrichment analysis by 1D annotation based on GOBP, showed enrichment of plant proteins associated with defence response, fatty acid ß-oxidation and glutathione metabolic process over time with enrichment in photosynthesis, chlorophyll and translation at the initial time points (FIG. 5C).

Next, assessment of A. tumefaciens protein production demonstrated a rapid and substantial increase in bacterial proteins with higher abundance over time (FIG. 5D). Evaluation of bacterial virulence factor production and motility, including VirB9, VirD2, VirH1, VirH2, FlaA and FlaB showed early elevated abundance of VirH1, FlaB and VirB9 by 3 dpi with VirH1 and FlaB decreasing over time compared to VirB9, which continues to increase, and VirH2, which has its highest production by 7 dpi (FIG. 5E). These findings are intriguing given the earlier induction of several bacterial virulence factors following bioreactor growth compared to shake flask, as supported by the previous ‘priming’ hypothesis, along with the inverse relationship between VirH1 and VirH2 between the growth conditions.

### Plant Metabolite Production Is Driven by Time and Not Bacterial Growth Conditions

Given the role of secondary metabolites in regulating plant health, the differences in metabolite production across time and in response to bacterial growth were quantified. Specifically, secondary metabolites regulate response to abiotic and biotic stress, including agroinfiltration, defence response to pathogen attack and induction of hypersensitive response to influence target protein production via molecular pharming (Drapal et al., 2021; Guy et al., 2008; Johnson et al., 2016). The analysis of the metabolome revealed 14 significant different metabolites across at least one tested condition (i.e. bioreactor versus shake flask, temporal consideration) with confirmation by tandem mass spectrometry and library matching (FIG. 6). These identified metabolites included six phenolic compounds (i.e. phenylacetaldehyde, phenolpropanoid 7-hydroxycoumarine, three cinnamamides feruloyl putrescine, feruloyltyramine, feruloyl O-methyldopamine, quinic acid derivative 5-p-coumaroylquinic acid), five terpenoids (i.e. monoterpene myrcene, a sesquiterpene, two terpene glycosides, terpene lactone), a jasmonic acid (JA) precursor (i.e. 12-Oxo phytodienoic acid [12ODPA]), a purine nucleobase (i.e. adenine) and an enzyme cofactor (i.e. riboflavin). All metabolites were significantly different between 0 and 7 dpi in plants infiltrated with either shake flask- or bioreactor-grown A. tumefaciens. A single metabolite, 7-hydroxycoumarine, showed a significant decrease in abundance at 7 dpi in plants infiltrated with shake flask-grown A. tumefaciens. Overall, metabolome profiling defined temporal differences in secondary metabolite production across pathways with a general increase over time but did not define bacterial growth-specific factors regulating production.

### Integration of Multi-OMICs Data Sets Reveal Modulation of Host Defence Responses Through Protein Abundance and Metabolite Production

Integrating proteomic and metabolomic data is an important approach to gain a comprehensive understanding of the molecular mechanisms and regulatory networks underlying biological systems (Bersanelli et al., 2016; Yan et al., 2017). The Kyoto Encyclopedia of Genes and Genomes (KEGG) database was searched to integrate the proteomic and metabolomics data, with plant proteins displaying significant differences in abundance from both shake flask and bioreactor growth conditions between 0 and 7 dpi. This analysis identified seven pathways with significantly different proteins and metabolites that, in general, correlated in production changes (Table 3). Of particular interest, the a-linolenic acid metabolism encompasses the JA pathway, which is related to plant resistance, prompting plant responses to external damage, pathogen infection and inducing resistance gene expression (Gupta et al., 2017; Ruan et al., 2019; FIG. 7). For a-linolenic acid metabolism pathway (12-OPDA detected), five proteins were identified within the pathway with allene oxide cyclase 4e (significantly different in bioreactor) and peroxisomal fatty acid ß-oxidation multifunctional protein (significantly different in shake flask) showing bacterial growth condition differences. Notably, the JA (a fatty acid-derived regulator) pathway contained two proteins involved in fatty acid ß-oxidation, biological process that showed a positive enrichment over time by 1D annotation in N. benthamiana infiltrated with both shake flask- and bioreactor-grown A. tumefaciens. These data support an overall theme of importance for these host defence response under the evaluated molecular pharming parameters.

The role of plant proteases in molecular pharming was further explored to build upon the discoveries of protein and metabolite signatures of N. benthamiana and A. tumefaciens that influence bacterial virulence and trastuzumab production. This work focused on apoplastic proteases with roles in protein degradation along the secretory pathway given the location of trastuzumab within the apoplast (Grosse-Holz et al., 2018a, 2018b). For instance, seven subtilisin-like proteases were identified with known roles in molecular pharming (Figueiredo et al., 2014; Puchol Tarazona et al., 2021) and three aspartic proteases, which act in the plant secretory pathway and are associated with plant defence due to biotic stress (Figueiredo et al., 2021). The characterized subtilisin-like proteases were deemed as validation of this approach and the role of the aspartic proteases was further explored, including aspartic proteinase A1-like; LOC107809122 (AP A1), aspartyl protease AED3-like; LOC107806163 (AED3-1), aspartyl protease AED3-like; LOC107828826 (AED3-2). To test the role of these aspartic proteases in trastuzumab production, short hairpin RNAs (shRNAs) were generated complementary to the target protein sequences. The shRNA silencing vectors were designed for each aspartic protease and an additional shRNA to target both AED3 proteases given high sequence similarity. All treatment groups were compared to the plant infiltrated with only the trastuzumab vector, lacking the shRNA vector. The results showed that agroinfiltration of N. benthamiana with A. tumefaciens possessing an shRNA plasmid targeting AED3-1 resulted in a significant increase (P-value=0.028) in trastuzumab yield (corresponding to a 22% increase in production) compared to the control (FIG. 8A). Notably, suppression of AED3-2 and AP A1 led to a significant reduction (P-value=0.011 and 0.025 respectively) in trastuzumab production compared to the control (losses of 21% and 18% respectively), and no significant difference (P-value=0.4374) for silencing of the combinatory AED2-1/2 proteases was observed (FIG. 8B). These data demonstrate the power of multi-OMICs profiling to define biological drivers of target protein production in molecular pharming and propose new strategies for improved production of trastuzumab.

### Discussion

This study investigated the impact of A. tumefaciens growth conditions and a temporal scale of N. benthamiana infection on host defence responses, bacterial virulence and trastuzumab production during the molecular pharming process. A comprehensive analysis of the proteome and metabolome over a time course of infection in consideration of distinct bacterial growth conditions was performed (i.e. bioreactor versus shake flask). The integration of proteomic and metabolomic data allowed for a comprehensive understanding of the molecular interactions identifying whole pathways simulated and repressed during infection. Overall, the multi-OMICs strategy proposes the benefits of bacterial bioreactor growth conditions on early production of the trastuzumab heavy and light chains, as well as potential for earlier and more frequent harvesting to improve final yields. Moreover, the impact of plant protease silencing on trastuzumab production was explored as a proof-of-concept strategy to further improve target protein production.

The influence of bacterial growth condition (bioreactor versus shake flask) on a time course of N. benthamiana infection was first evaluated. A significant increase in production of the trastuzumab heavy and light chains at the early time point (i.e. 1 dpi) was observed following bacterial bioreactor growth compared to shake flask growth (i.e. 3 dpi). Previously, it was hypothesized that bacterial bioreactor growth ‘primes’ Agrobacterium for infection by observing a significant increase in ABC transporter proteins and proteins involved in iron update associated with the bioreactor that promote bacterial survivability and adaptability (Prudhomme et al., 2021a). Data in the present study support this hypothesis through detection of an ABC transporter and outer membrane proteins (BamA and Omp) with significantly increased abundance at 1 dpi and three ABC transporters and FlaB at 3 dpi, each associated with bacterial bioreactor growth. Notably, outer membrane proteins play vital roles in diverse essential cellular processes, including nutrient update (Klebba and Newton, 1998), protein secretion and adhesion (Lauber et al., 2018; Stubenrauch et al., 2016), as well as membrane protein insertion and protein secretion reactions in bacteria (Baud et al., 2014; Doyle and Berstein, 2019). ABC transporters play an essential role in nutrient acquisition within the hostile host environment to promote bacterial survivability (Tanaka et al., 2018), while the roles of flagellar proteins and bacterial virulence associated with surface attachment have been explored (Oberpichler et al., 2008; Prudhomme et al., 2021b). Significant alterations of bacterial proteins by GOBP associated with motility and phosphorelay signal transduction driven by the bioreactor growth conditions was also defined (Prudhomme et al., 2021a). These results indicate that bioreactor adaptability and increased virulence at early timepoints in plant infection lead to enhanced production of trastuzumab heavy and light chains at early stages following infiltration.

In the present study, the CFU counts at the collection OD₆₀₀ₙₘ was assessed following bacterial growth in bioreactor or shake flask. A higher CFU count was observed for bacteria grown in shake flasks compared to the bioreactor, which likely alters the state of the bacteria associated with each growth condition. Proteome profiling on these sample sets were previously performed and distinct differences in the bacterial proteomes corresponding to growth condition were observed, including enrichment of proteins associated with metabolic and biosynthetic processes following shake flask growth (Prudhomme et al., 2021a). These findings suggest that even with the higher CFUs, the bacterial cells in shake flasks are still metabolically active at the collection time points. Critically, the potential confounding factors, such as timing of media component depletion and the impact of residual media components on gene expression in planta, were not assessed following agroinfiltration with A. tumefaciens from bioreactor versus shake flask growth. It is acknowledged that such differences may influence plant response to the bacterium; however, minimal impact on altered plant protein production is anticipated given the dilution of the bacteria and culture media within agroinfiltration media prior to plant infection (Prudhomme et al., 2021b). Moreover, the proteomic profiling of agroinfiltration media following inoculation with shake flask- or bioreactor-grown bacteria defined clear distinction between bacterial proteomes dependent upon growth conditions, but consistency in protein abundance trends towards increased motility and virulence of bioreactor-grown bacteria were observed. Together, these findings further support the unique properties of growing bacteria under shake flask or bioreactor conditions, although, there are still some components of bacterial growth that were not accounted for following agroinfiltration.

Effector-triggered immunity (ETI) constitutes a pivotal component of plant innate immunity, providing crucial protection against pathogen infections (Zhang et al, 2018). Upon pathogen invasion, plants activate the induction mechanism of ETI, resulting in the accumulation of pathogenesis-related (PR) proteins (Mazumder et al., 2013). The effect of bacterial growth conditions showed a notable difference in how N. benthamiana responded to infection. For instance, production of plant defence response proteins showed a significant increase at 1 dpi following agroinfiltration with shake flask-grown bacteria, while a significant increase in plant defence-associated proteins following agroinfiltration with bioreactor-grown bacteria was not detected until 3 dpi. Notably, between 0 and 1 dpi, plants infiltrated with shake flask-grown bacteria showed a significant increase in two PR1 proteins and Suppressor of G2 allele of SKP1 (SGT1). SGT1 is required for disease resistance mediated by resistance proteins and plays a role in hypersensitive cell death response (Azevedo et al., 2002; Kim et al., 2014; Peart et al., 2002). Between 0 and 3 dpi, this response was expanded to include two osmotin-like proteins (OLPs), and between 0 and 7 dpi, and the addition of two PR proteins and 1 OLP was observed. OLPs are proteins belonging to the

PR-5 family accumulating in response to abiotic and biotic stresses and lyse the plasma membrane of invading pathogens (Anil Kumar et al., 2015). Between 0 and 1 dpi in plants infiltrated with bioreactor-grown bacteria there was no significant increase in PR proteins, SGT1 or OLPs. Between 0 and 3 dpi and 0 and 7 dpi bioreactor-grown A. tumefaciens shows comparable numbers of significantly different PR, SGT1 and OLPs to bacteria grown within a shake flask, indicating the plant immune response quickly “catches up” after being evaded.

Another critical observation was the differences in the activation of catabolic processes in shake flask versus bioreactor infiltrated plants. For example, differences in catabolic proteins showed an increase in the number of proteins involved in proteolysis and production of reactive oxygen species (ROS) in the bioreactor compared to cell wall catabolismand phospholipase production (i.e. involved in defence signalling in plants) in the shake flask (Canonne et al., 2011). Following successful pathogen recognition, one of the initial cellular responses is the generation of ROS catalysed by peroxidases (Auh and Murphy, 1995; Doke, 1983; van Loon et al., 2008). Challenges associated with yield and purity of proteins produced by agroinfiltration include unintended proteolysis, which have restricted industrial applications (Grosse-Holz et al., 2018a). The experimental silencing of select plant aspartic proteases with differential production dependent upon bacterial growth conditions, validates the role of proteolytic activity on degradation of trastuzumab in the tested system and indicates a new strategy for improved target protein production. These findings indicate that bacterial growth conditions can modulate specific cellular processes in the plant, impacting its defence response and target protein production.

The analysis of the plant metabolome revealed temporal differences in secondary metabolite production, irrespective of bacterial growth conditions. Fourteen metabolites were significantly different across time, including phenolic compounds, terpenoids, a JA precursor, an enzyme cofactor and purine nucleobase. Several of the identified phenolic compounds are cinnamamides (hydroxycinnamic acids [HCAs] and hydroxycinnamoyl-quinic acids [HCQAs]), including 7-hydroxycoumarine, feruloylputrescine, feruloyltyramine, feruloyl o-methyldopamine and 5-p-coumaroylquinic acid. HCAs and HCQAs are involved in the stress response in plants, especially at the site of wounding and exhibit antimicrobial activity against many bacterial species (Bassard et al., 2010; Liu et al., 2011; Valanciene and Malys, 2022). These compounds showed a predicted increase in abundance over the 7-day time course of infection. Surprisingly, 7-hydroxycoumarine, which only showed a significant difference between 0 and 7 dpi in plants infiltrated with shake flask-grown bacteria, decreased in abundance. Plant terpenoids play essential roles in direct and indirect defence mechanisms and function as airborne signals detectable by undamaged systemic parts of the same plant as well as neighbouring plants (Heil and Silva Bueno, 2007; Karban et al., 2000). In response to these volatile signals, plants activate defence genes and produce numerous other secondary metabolites (Shulaev et al., 1997; Sugimoto et al., 2014).

JA and its precursors, such as 12-OPDA, play crucial roles in regulating various physiological processes involved in plant growth, development and the response to both biotic and abiotic stresses. The signalling pathways mediated by JA are primarily associated with plant defence mechanisms, enabling plants to respond to external damage caused by factors like mechanical stress, herbivores, insects and pathogen infections (Kazan and Manners, 2008; McConn et al., 1997). As a result, JA triggers the expression of resistance genes, leading to the induction of plant resistance (Schenk et al., 2000). While many metabolites involved in host defence response were identified, no specific metabolites were found to be regulated by bacterial growth conditions. This differs in comparison to the many protein-level changes observed between shake flask and bioreactor infiltrated plants. Since variations in metabolite abundance reflect the flow of chemicals resulting from diverse biochemical reactions, molecular mechanisms and biological pathways mediated by hundreds of proteins, it is possible the metabolome profile only shows the response to general A. tumefaciens infection but does not tease apart the niche differences imparted by the bacterial growth condition. Additionally, this study profiled metabolites extracted with an organic solvent (50% ACN) processed in positive ion mode on the mass spectrometer, which limits the number of identifications. Many LC-MS ‘global’ metabolomics experiments run organic and aqueous extractions with several solvents to increase the metabolome coverage.

Integrating proteomic and metabolomic data provides a comprehensive understanding of the complex molecular interactions and regulatory networks underlying biological systems. This study successfully identified circumstances with significant changes in both protein abundance and metabolite production for seven biosynthetic pathways. For instance, in the a-linolenic acid metabolism pathway, which includes the JA pathway, the JA precursor, 12-OPDA and six enzymes involved in fatty acid ß-oxidation that catalyse the conversion of 12-OPDA into JA were identified (Germain et al., 2001; Shiraku et al., 2021; Zdyb et al., 2018). The 1D annotation enrichment analysis based on GOBP also showed significant positive enrichment of proteins associated with fatty acid ß-oxidation in plants infiltrated with both shake flask-and bioreactor-grown bacteria. Besides its role in JA production, ß-oxidation plays an essential role in the destruction of reserve triglycerides and growth in photosynthesis limiting situations and in response to stress (Graham, 2008; Kunz et al., 2009). A by-product of fatty acid ß-oxidation is ROS, which, as mentioned above, are also produced in response to pathogen recognition.

### Conclusion

The findings of this study have important implications for optimizing target protein production in molecular pharming. For instance, defining the interplay between bacterial growth conditions, host defence responses and target protein production can guide the creation of more efficient and effective production strategies. The findings in this study support the potential for earlier production of trastuzumab following A. tumefaciens growth in a bioreactor based on bacterial ‘priming’ for enhanced infectivity and evasion of early plant defences. In addition, an important role for aspartic protease AED3-1 in modulating trastuzumab production was observed. Moreover, the integration of proteomic and metabolomic analyses provides a valuable approach for unravelling the underlying molecular mechanisms (e.g. precursors of JA, defence proteins) and identifying key pathways (e.g. silencing of aspartic proteases) targeted for optimization. Taken together, opportunities for improvements to the molecular pharming pipeline appear dynamic and a combinatory approach of defined bacterial growth conditions (i.e. bioreactor), earlier and sequential harvesting (i.e. 3 dpi), and silencing of select proteases (i.e. AED3-1) increases production of trastuzumab.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present description is found to be defined differently in a document incorporated herein by reference, the definition provided herein is to serve as the definition for the term.

TABLE 1
List of identified proteins containing
GOBP defense response, [GO: 0006952].

| Protein names | Protein ID |
|---|---|
| Acidic endochitinase Q (EC 3.2.1.14) (Pathogenesis- | P17514 |
| related protein Q) (PR-Q) | |
| basic endochitinase-like | A0A1S3ZVW5 |
| Basic form of pathogenesis-related protein 1 (PRP 1) | P11670 |
| basic form of pathogenesis-related protein 1-like | A0A1S4AL11 |
| Chitinase 134 (basic endochitinase) | Q9ZWS3 |
| chitinase-like protein 1 | A0A1S4CA37 |
| cytochrome P450 81F3-like | A0A1S4CQM2 |
| defensin J1-2-like | A0A1S3Y6I9 |
| defensin-like protein P322 | A0A1S4BPM7 |
| endochitinase 3-like | A0A1S3YVP8 |
| Endochitinase B (CHN-B) (EC 3.2.1.14) | P24091 |
| endochitinase EP3-like | A0A1S3YRD6 |
| Glucan endo-1,3-beta-glucosidase, acidic isoform GL161 | P52398 |
| (EC 3.2.1.39) | |
| kirola-like | A0A1S3Y166 |
| MLP-like protein 423 | A0A1S4BS12 |
| Osmotin | P14170 |
| osmotin-like protein | A0A1S3ZK42 |
| osmotin-like protein | A0A1S4CYY0 |
| Pathogenesis-related protein 1A (PR-1A) | P08299 |
| Pathogenesis-related protein PR10 (pathogenesis-related | H2ESL0 |
| protein STH-2-like) | |
| Pathogenesis-related protein PR-4A | P29062 |
| pathogenesis-related protein R minor form | A0A1S3XXM2 |
| pathogenesis-related protein STH-2-like | A0A1S3XZH5 |
| putative disease resistance protein RGA1 | A0A1S4D196 |
| putative disease resistance RPP13-like protein 3 | A0A1S4D9T9 |
| Putative PR-10 type pathogenesis-related protein (kirola- | E3W9P7 |
| like) | |
| Ribulose bisphosphate carboxylase small subunit, | P69249 |
| chloroplastic (RuBisCO small subunit) (TSSU3-8) | |
| single-stranded DNA-binding protein WHY1, | A0A1S4ACQ8 |
| chloroplastic-like | |
| S-norcoclaurine synthase 1-like | A0A1S4DNH2 |
| Suppressor of G2 allele of SKP1 (protein SGT1 | A0A1S4BMU1 |
| homolog) | |
| thioredoxin-like protein CITRX1, chloroplastic | A0A1S3YEW3 |
| wound-induced protein WIN1-like | A0A1S4A6F6 |

TABLE 2
List of identified apoplastic proteases.

| Protein names | Protein ID |
|---|---|
| aspartic proteinase A1-like | A0A1S4BJV8 |
| aspartyl protease AED3-like | A0A1S4BAC6 |
| aspartyl protease AED3-like | A0A1S4DE48 |
| Subtilisin-like protease (subtilisin-like protease SBT1.7) | A9XG41 |
| subtilisin-like protease SBT1.4 | A0A1S3YHX9 |
| subtilisin-like protease SBT1.4 | A0A1S4AAX6 |
| subtilisin-like protease SBT1.8 | A0A1S3YPY3 |
| subtilisin-like protease SBT1.8 | A0A1S4CQL6 |
| subtilisin-like protease SBT1.9 | A0A1S3ZQR1 |
| subtilisin-like protease SBT3.4 | A0A1S4DJT1 |

TABLE 3
Integration of proteomic and metabolomic datasets

| Metabolite | Protein | | | Fold difference (log₂) | |
|---|---|---|---|---|---|
| Name pathway KEGG ID | Uniprot ID | KEGG ID | Protein name | Flask | Bioreactor |
| 12-oxo [symbol] acid | [symbol] | nta: [symbol] | [symbol] | — | 1.83 |
| Alpha-Linolenic acid metabolism | [symbol] | nta: [symbol] | [symbol] | 2.37 | 2.32 |
| [symbol] | [symbol] | nta: [symbol] | [symbol] | 2.12 | 1.69 |
| | | | a-like | | |
| | [symbol] | nta: [symbol] | Acyl-coenzyme A oxidazse 3, peroxisomal-like | 1.53 | 1.48 |
| | [symbol] | nta: [symbol] | [symbol] | 0.84 | — |
| | | | [symbol] | | |
| Adenine | [symbol] | nta: [symbol] | HD domin-containing protein 2-like | — | 1.03 |
| Purine metabolism [symbol] | [symbol] | nta: [symbol] | [symbol] | — | 1.59 |
| | [symbol] | nta: [symbol] | Adenylate kinase 4-like | 1.05 | 1.53 |
| | [symbol] | nta: [symbol] | [symbol] | — | 1.38 |
| Phenylacetaldehyde | [symbol] | nta: [symbol] | Aspartate aminotransferase, cytoplasmic | 1.66 | 2.19 |
| Phenylalanine metabolism [symbol] | [symbol] | nta: [symbol] | [symbol] | 1.35 | 1.00 |
| | [symbol] | nta: [symbol] | Aspartate aminotransferase, mitochondrial-like | 0.68 | 1.63 |
| | [symbol] | nta: [symbol] | Aspartate aminotransferase, cytoplasmic | 0.77 | 1.82 |
| | [symbol] | nta: [symbol] | Bifunctional aspartate aminotransferase and glutamate/ | — | 1.17 |
| | | | aspartate-prephenate aminotransferase-like | | |
| | [symbol] | nta: [symbol] | [symbol] | 0.76 | 1.17 |
| | [symbol] | nta: [symbol] | [symbol] | — | 1.69 |
| 7-Hydroxycoumarine | [symbol] | nta: [symbol] | [symbol] | −0.79 | — |
| Biosynthesis of various plant secondary | [symbol] | nta: [symbol] | [symbol] | −1.45 | — |
| metabolites [symbol] | [symbol] | nta: [symbol] | [symbol] | −1.65 | −1.04 |
| | [symbol] | nta: [symbol] | [symbol] | −0.88 | −0.66 |
| [symbol] | [symbol] | nta: [symbol] | Aspartate aminotransferase, cytoplasmic | 1.66 | 2.19 |
| Arginine and proline metabolism | [symbol] | nta: [symbol] | Aldehyde dehydrogenase | 0.97 | 1.18 |
| [symbol] | [symbol] | nta: [symbol] | Delta-1-pyrroline-5-carboxylate dehydrogenase 12A1, | 0.89 | 0.96 |
| | | | mitochondrial-like | | |
| | [symbol] | nta: [symbol] | Aspartate aminotransferase, mitochondrial-like | 0.68 | 1.63 |
| | [symbol] | nta: [symbol] | Pyrroline-5-carboxylate reductase-like | 0.69 | — |
| | [symbol] | nta: [symbol] | Aldehyde dehydrogenase family 7 member [symbol] | 2.82 | 2.35 |
| | [symbol] | nta: [symbol] | Aspartate aminotransferase, cytoplasmic | 0.77 | 1.82 |
| | [symbol] | nta: [symbol] | Aspartate aminotransferase, chloroplastic-like | 0.76 | 1.17 |
| | [symbol] | nta: [symbol] | [symbol] ; spermidine synthase-like | 0.94 | — |
| 5-p- [symbol] acid | [symbol] | nta: [symbol] | TOBPAL1, phenylalanine ammonia-lyase | 1.35 | 1.00 |
| Phenylpropanoid biosynthesis [symbol] | [symbol] | nta: [symbol] | Peroxidase; lignin-forming anionic peroxidase-like | 1.73 | — |
| | [symbol] | nta: [symbol] | Peroxidase 21-like | 2.01 | 1.90 |
| | [symbol] | nta: [symbol] | 4CL1; 4-coumarate-CoA ligase 1 | 0.85 | — |
| | [symbol] | nta: [symbol] | Peroxidase 72-like | — | 2.76 |
| | [symbol] | nta: [symbol] | [symbol] | 2.84 | — |
| | [symbol] | nta: [symbol] | [symbol] | 4.18 | 3.43 |
| | [symbol] | nta: [symbol] | Peroxidase P7-like | 1.62 | 1.81 |
| | [symbol] | nta: [symbol] | Peroxidase, peroxidase P7-like | — | 2.37 |
| | [symbol] | nta: [symbol] | [symbol] | — | 1.63 |
| Myrcene and other terpenes | [symbol] | nta: [symbol] | [symbol] | 1.37 | 1.96 |
| Terpenoid backbone biosynthesis | [symbol] | nta: [symbol] | [symbol] | — | 0.75 |
| [symbol] | | | chloroplastic-like | | |
| | [symbol] | nta: [symbol] | Hydroxymethylglutaryl-CoA synthase-like | 2.29 | — |
| | [symbol] | nta: [symbol] | fps; farnesyl pyrophosphate synthase 1-like | 0.90 | 1.05 |
| | [symbol] | nta: [symbol] | [symbol] | 1.84 | — |
| | [symbol] | nta: [symbol] | [symbol] | 1.64 | 1.34 |
| | [symbol] | nta: [symbol] | [symbol] | — | 1.41 |

[symbol]

indicates data missing or illegible when filed

Sequences

SEQ ID NO: 1
Amino acid sequence, aspartic protease AED3-1, Nicotiana tabacum
MEGSIIFLLSLLLFFTLAKGLTNPKCGSSFPQADKGSTLQVLHVNSPCSPLRNNAPQSWV
DTVLQMQSKDQARLDLFASLVAGRSFVPIASGRQVIQSPTYIVRAKIGTPPQTLLVAVDN
SNDVAWFPCSGCVGCSSTVFASDKSTTFKNVSCGAAQCSQVPNPTCGGSSCGFNLTYG
GSSIAANLSQDTLTLATDAVPSYTFGCVQKATGSSAPPQGLLGLGRGPLSFLSQTQSHYQ
STFSYCLPSYKSPNFSGTLRLGPNGQPKRIKTTQLLRNPRRSSFYYVNLVGVKVGRRIVDI
PPSALAFNPSTGAGTIIDSGTVFTRLVEPAYTAVRNEFRRRMGRNTTVTSLGGFDTCYTV
PITIPTITLMFAGMNVTLPQDNFLIRSSSSSTTCLAMAASPADPVNSVLNVIANWQQQNHR
FLFDVPNSKLGVARETCS
SEQ ID NO: 2
shRNA sequence
CCGGUGUUGGUUGCUGUGGAUAAUAUCAAGAGUAUUAUCCACAGCAACCAACAUUU
UUG
SEQ ID NO: 3
DNA, forward sequence encoding the shRNA of SEQ ID NO: 2
lowercase underlined are the cloning sites; uppercase encodes the shRNA; uppercase
bold are the flanking sequences; uppercase bold underlined is the loop sequence.
aattaagcttCCGGTGTTGGTTGCTGTGGATAATATCAAGAGTATTATCCACAGCAACCAA
CATTTTTGgagctcatat
SEQ ID NO: 4
DNA, reverse complement of SEQ ID NO: 3
atatgagctcCAAAAATGTTGGTTGCTGTGGATAATACTCTTGATATTATCCACAGCAACCA
ACACCGGaagcttaatt
SEQ ID NO: 5
Genomic DNA sequence of aspartic protease AED3-1, Nicotiana tabacum
taaaccacac caaaagccaa agtactctca caagccaatg gagggctcta tcattttctt actatccctt ctcttatttt
tcactctagc caaagggcta accaacccca aatgtggcag cagctttccc caagcagaca aaggctcaac
actacaagtc ctacatgtga acagcccttg ctctcctctt aggaacaatg ctccccagtc atgggttgat actgtcctcc
aaatgcagtc caaagaccaa gccagacttg atttgtttgc tagtcttgtg gctgggagat cttttgttcc aattgcttcg
ggaagacaag ttatacagag cccaacttac atagtgaggg ccaagattgg aaccccacct caaaccttgt
tggttgctgt ggataatagc aatgatgttg cttggttccc ttgcagtggt tgtgttggtt gctcctctac tgtttttgca
tcagacaagt ccaccacttt caagaatgtt agctgtggag ctgcacaatg cagccaggta ccccattata cctttcaaac
caactttatt atacaccttt cttacatttt actctctccg tttttttta taaaaatgcc atttttgtct cgacataaaa
tttaagaagt
aaaaaaaggc ttttgacatg tattaaagtg tccttgtaac ttgtaaccat atcactacat ctctttagct ataagagttg
tataggaagt ttaatgtaaa agagttcatg aaggtgttat tcttgttaaa ataaattata atggaaagag tgtcagtaca
aaatggaatg gaggaacagc attttgaaag atttcctaat ttcctaaact agtttttcgt gcatttaact tctaatgttg
aagtactaga aattatctac atttctgcag cgatagcata tcaaccacat gttttttata tactactagt agtatataag
agcacaccta ccacatgtaa cgctttctct gtcttcaatt taatgatcct attcggctca aaataggctt agagcacttc
gtataaatag gattaaacag ctttcaacat aataatctat taagcacaac tttcaggata agaaaatgat cataatttaa
gaaaagctag tgaatttttt aaaataggaa acgagcttcg tatggcgcgt gggaactgaa aagttgtttc ctctttcctg
gctttgtcga cattggagca attagcggta gagttatccg agataactaa tcccgtgacc cttagtcaca tgatttggtc
aacattctca ttccacaaaa gaacatcatt tatttgtaag acacttttat gtcgctaaaa tataagatgt tactggtaaa
atggacaata ggaattccaa gtataaagca caataactag gtttcagtat caaataagtt cagctggact atatgaaatt
ttacacaaca agatttattt gtgtatatgc taattattac taaaaaaata tttttctcag gtaccaaatc ccacatgcgg
tggcagcagc tgcggcttca acctaaccta cggcggctcc agcatagctg caaatctctc acaagacaca
ttgacactcg ccactgacgc cgtgccttca tatacctttg gttgtgtaca aaaggccacc ggcagctccg cgccacccca
ggggctatta ggtttgggtc gaggcccatt gtcatttttg tcccaaaccc aaagccatta ccagtctaca ttctcctact
gtttgcccag ttacaagtct cccaactttt ccggcacact caggttgggc ccaaatggcc agcccaaaag
gattaagaca actcaattgc taagaaaccc aaggagatct tccttttatt atgtcaactt ggtcggagtt aaagtcggcc
ggagaatcgt tgacatccct cccagtgctt tggctttcaa cccttccacc ggtgccggca ccataattga ttcgggtatt
taatttaaca atgatcaata tatctgtaat tataattcta tgtaccaatt tttgctaaaa ggcattgtca atcgacagga
acggtattca cgaggctagt ggagccagcg tatacggcag tgaggaacga gtttaggagg agaatgggaa
ggaacacgac cgtgacaagc cttggcggat tcgacacttg ctacaccgtc cccattacaa ttccaacaat
aacgctgatg tttgcgggca tgaacgtgac gctgccgcaa gacaacttct taatccgcag cagttccagc agtacaactt
gcctcgctat ggctgcttcc ccagccgacc ctgtcaattc cgtcctcaac gtcatcgcca actggcagca acagaatcac
cgcttcctct ttgacgttcc taattctaag ctcggcgttg ctcgtgaaac ctgcagctga agtactgctt ttggatttta
atgggatttt ctccttttgg ctctttccgt tttttctttt ttcttaacgt ggggaattaa ttaattatta gagatagttt
tcacggtttg
gcactttgat attgtcggtg cattttgctg ttttggattt atcttatctg tgtcacgggt tgaaagttac tacacctgtt
tccaactatt atcctcctat ttatttcttg gtttccccac cttgaaggaa aacagaaaat gattgctcgt ccattccagt t
SEQ ID NO: 6
P19 suppressor used in vector pPFC0058
ATGGAAAGGGCTATTCAGGGAAATGATGCTAGAGAGCAGGCTAATTCTGAAAGATGG
GATGGTGGATCTGGTGGAACTACTTCTCCATTCAAGCTTCCAGATGAGTCTCCATCTT
GGACTGAGTGGAGGCTTCATAACGATGAGACTAACTCCAATCAGGATAACCCACTCG
GATTCAAAGAATCTTGGGGATTCGGAAAGGTTGTGTTCAAGCGTTACCTTAGGTATGA
TAGGACTGAGGCTTCACTTCATAGGGTTCTCGGATCTTGGACTGGTGATTCTGTTAAC
TACGCTGCTTCTCGTTTTTTTGGATTCGATCAGATCGGATGCACTTACTCTATTAGGTT
CAGGGGAGTGTCTATTACTGTTTCTGGTGGATCTAGGACTCTTCAACACCTTTGCGAG
ATGGCTATTAGGTCTAAGCAAGAGCTTCTTCAGCTTGCTCCAATTGAGGTTGAGTCTA
ACGTTTCAAGAGGATGTCCAGAAGGTACTGAGACTTTCGAGAAAGAATCCGAGTGA

### CITATIONS FOR REFERENCES REFERRED TO IN THE SPECIFICATION

Aebersold, R. and Mann, M. (2016) Mass-spectrometric exploration of proteome structure and function. Nature, 537, 347-355.
Anil Kumar, S., Hima Kumari, P., Shravan Kumar, G., Mohanalatha, C. and Kavi Kishor, P. B. (2015) Osmotin: a plant sentinel and a possible agonist of mammalian adiponectin. Front. Plant Sci. 6, 163.
Ashburner, M., Ball, C. A., Blake, J. A., Botstein, D., Butler, H., Cherry, J. M. et al. (2000) Gene Ontology: tool for the unification of biology. Nat. Genet. 25, 25-29.
Auh, C. K. and Murphy, T. M. (1995) Plasma membrane redox enzyme is involved in the synthesis of O2— and H2O2by Phytophthora elicitor-stimulated rose cells. Plant Physiol. 107, 1241-1247.
Altschul, S. F., W. Gish, W. Miller, E. W. Myers and D. J. Lipman, 1990 Basic local alignment search tool. J Mol Biol 215:403-410.
Altschul, S. F., Madden, T. L., Schaffer, A. A., Zhang, J., Zhang, Z., Miller, W. and Lipman, D. J. (1997) Gapped BLAST and PSI-BLAST: a new generation of protein database search programs. Nucleic acids research 25, 3389-3402.
Azevedo, C., Sadanandom, A., Kitagawa, K., Freialdenhoven, A., Shirasu, K. and Schulze-Lefert, P. (2002) The RAR1 interactor SGT1, an essential component of R gene-triggered disease resistance. Science, 295, 2073-2076.
Bassard, J.-E., Ullmann, P., Bernier, F. and Werck-Reichhart, D. (2010) Phenolamides: bridging polyamines to the phenolic metabolism. Phytochemistry, 71, 1808-1824.
Baud, C., Gu erin, J., Petit, E., Lesne, E., Dupr e, E., Locht, C. and Jacob-Dubuisson, F. (2014) Translocation path of a substrate protein through its Omp85 transporter. Nat. Commun. 5, 5271.
Benjamini, Y. and Hochberg, Y. (1995) Controlling the false discovery rate: a practical and powerful approach to multiple testing. J. R. Stat. Soc. B. Methodol. 57, 289-300.
Berger S A, “Increasing protein production by increasing ABC50 expression or activity,” U.S. Pat. No. 8,691,529, Apr. 19, 2012.
Bersanelli, M., Mosca, E., Remondini, D., Giampieri, E., Sala, C., Castellani, G. and Milanesi, L. (2016) Methods for the integration of multi-omics data: mathematical aspects. BMC Bioinformatics, 17, 15.
Buyel, J. F., Twyman, R. M. and Fischer, R. (2017) Very-large-scale production of antibodies in plants: the biologization of manufacturing. Biotechnol. Adv. 35, 458-465.
Buyel, J. F., St€oger, E. and Bortesi, L. (2021) Targeted genome editing of plants and plant cells for biomanufacturing. Transgenic Res. 30, 401-426.
Canonne, J., Froidure-Nicolas, S. and Rivas, S. (2011) Phospholipases in action during plant defense signaling. Plant Signal. Behav. 6, 13-18.
Chen, Q. and Davis, K. R. (2016) The potential of plants as a system for the development and production of human biologics. F1000Res. 5, 912.
Cox, J. and Mann, M. (2008) MaxQuant enables high peptide identification rates, individualized p.p.b.-range mass accuracies and proteome-wide protein quantification. Nat. Biotechnol. 26, 1367-1372.
Cox, J. and Mann, M. (2012) 1D and 2D annotation enrichment: a statistical method integrating quantitative proteomics with complementary highthroughput data. BMC Bioinformatics, 13, S12.
Cox, J., Neuhauser, N., Michalski, A., Scheltema, R. A., Olsen, J. V. and Mann,. (2011) Andromeda: a peptide search engine integrated into the MaxQuant environment. J. Proteome Res. 10, 1794-1805.
Cox, J., Hein, M. Y., Luber, C. A., Paron, I., Nagaraj, N. and Mann, M. (2014) Accurate proteome-wide label-free quantification by delayed normalization and maximal peptide ratio extraction, termed MaxLFQ. Mol. Cell. Proteomics, 13, 2513-2526.
Doke, N. (1983) Involvement of superoxide anion generation in the hypersensitive response of potato tuber tissues to infection with an incompatible race of Phytophthora infestans and to the hyphal wall components. Physiol. Plant Pathol. 23, 345-357.
Doyle, M. T. and Bernstein, H. D. (2019) Bacterial outer membrane proteins assemble via asymmetric interactions with the BamA b-barrel. Nat. Commun. 10, 3358.
Drapal, M., Enfissi, E. M. A. and Fraser, P. D. (2021) Metabolic effects of agroinfiltration on N. benthamiana accessions. Transgenic Res. 30, 303-315.
Figueiredo, A., Monteiro, F. and Sebastiana, M. (2014) Subtilisin-like proteases in plant-pathogen recognition and immune priming: a perspective. Front. Plant Sci. 5, 739.
Figueiredo, L., Santos, R. B. and Figueiredo, A. (2021) Defense and offense strategies: the role of aspartic proteases in plant-pathogen interactions. Biology (Basel), 10, 75.
Fischer, R. and Buyel, J. F. (2020) Molecular farming-the slope of enlightenment. Biotechnol. Adv. 40, 107519.
Garabagi, F., Gilbert, E., Loos, A., McLean, M. D. and Hall, J. C. (2012a) Utility of the P19 suppressor of gene-silencing protein for production of therapeutic antibodies in Nicotiana expression hosts. Plant Biotechnol. J. 10, 1118-1128.
Garabagi, F., McLean, M. D. and Hall, J. C. (2012b) Transient and stable expression of antibodies in nicotiana species. Methods Mol. Biol. 907, 389-408.
Geddes-McAlister, J., Prudhomme, N., Dutierrez Gongora, D., Cossar, D. and McLean, M. D. (2022) The emerging role of mass spectrometry-based proteomics in molecular pharming. Curr. Opin. Chem. Biol. 68, 102133.
Germain, V., Rylott, E. L., Larson, T. R., Sherson, S. M., Bechtold, N., Carde, J., Bryce, J. H. et al. (2001) Requirement for 3-ketoacyl-CoA thiolase-2 in peroxisome development, fatty acid b-oxidation and breakdown of triacyIglycerol in lipid bodies of Arabidopsis seedlings. Plant J. 28, 1-12.
Gershenzon, J. and Dudareva, N. (2007) The function of terpene natural products in the natural world. Nat. Chem. Biol. 3, 408-414.
Graham, I. A. (2008) Seed storage oil mobilization. Annu. Rev. Plant Biol. 59, 115-142.
Grohs, B. M., Niu, Y., Veldhuis, L. J., Trabelsi, S., Garabagi, F., Hassell, J. A., McLean, M. D. et al. (2010) Plant-produced trastuzumab inhibits the growth of HER2 positive cancer cells. J. Agric. Food Chem. 58, 10056-10063.
Grosse-Holz, F., Kelly, S., Blaskowski, S., Kaschani, F., Kaiser, M. and van der Hoorn, R. A. L. (2018a) The transcriptome, extracellular proteome and active secretome of agroinfiltrated Nicotiana benthamiana uncover a large, diverse protease repertoire. Plant Biotechnol. J. 16, 1068-1084.
Grosse-Holz, F., Madeira, L., Zahid, M. A., Songer, M., Kourelis, J., Fesenko, M., Ninck, S. et al. (2018b) Three unrelated protease inhibitors enhance accumulation of pharmaceutical recombinant proteins in Nicotiana benthamiana. Plant Biotechnol. J. 16, 1797-1810.
Gupta, A., Hisano, H., Hojo, Y., Matsuura, T., Ikeda, Y., Mori, I.C. and Senthil-Kumar, M. (2017) Global profiling of phytohormone dynamics during combined drought and pathogen stress in Arabidopsis thaliana reveals ABA and JA as major regulators. Sci. Rep. 7, 4017.
Guy, C., Kopka, J. and Moritz, T. (2008) Plant metabolomics coming of age. Physiol. Plant. 132, 113-116.
Hager, K. J., P erez Marc, G., Gobeil, P., Diaz, R. S., Heizer, G., Llapur, C., Makarkov, A. I. et al. (2022) Efficacy and safety of a recombinant plant-based adjuvanted Covid-19 vaccine. N. Engl. J. Med. 386, 2084-2096.
Haydon, I. (2017) Biologics: the pricey drugs transforming medicine. The Conversation. https://theconversation.com/biologics-the-pricey-drugs-transformingmedicine-80258Heil, M. and Silva Bueno, J. C. (2007) Within-plant signaling by volatiles leads to induction and priming of an indirect plant defense in nature. Proc. Natl Acad. Sci. USA, 104, 5467-5472.
Hood, E. E., Gelvin, S. B., Melchers, L. S. and Hoekema, A. (1993) New Agrobacterium helper plasmids for gene transfer to plants. Transgenic Res. 2, 208-218.
Houdelet, M., Galinski, A., Holland, T., Wenzel, K., Schillberg, S. and Buyel, J. F. (2017) Animal component-free Agrobacterium tumefaciens cultivation media for better GMP-compliance increases biomass yield and pharmaceutical protein expression in Nicotiana benthamiana. Biotechnol. J. 12, 1600721.
Johnson, C. H., Ivanisevic, J. and Siuzdak, G. (2016) Metabolomics: beyond biomarkers and towards mechanisms. Nat. Rev. Mol. Cell Biol. 17, 451-459.
Kanehisa, M. and Sato, Y. (2019) KEGG Mapper for inferring cellular functions from protein sequences. Protein Sci. 29, 28-35.
Kantardjieff, A. and Zhou, W. (2013) Mammalian cell cultures for biologics manufacturing. Adv. Biochem. Eng. Biotechnol. 139, 1-9.
Karban, R., Baldwin, I. T., Baxter, K. J., Laue, G. and Felton, G. W. (2000) Communication between plants: induced resistance in wild tobacco plants following clipping of neighboring sagebrush. Oecologia, 125, 66-71.
Karlin, S., and S. F. Altschul, 1990 Methods for assessing the statistical significance of molecular sequence features by using general scoring schemes. Proc Natl Acad Sci U S A 87:2264-2268.
Karlin, S. and Altschul, S. F. (1993) Applications and statistics for multiple high-scoring segments in molecular sequences. Proceedings of the National Academy of Sciences 90, 5873-5877.
Kazan, K. and Manners, J. M. (2008) Jasmonate signaling: toward an integrated view. Plant Physiol. 146, 1459-1468.
Kim, N. H., Kim, D. S., Chung, E. H. and Hwang, B. K. (2014) Pepper suppressor of the G2 allele of skp1 interacts with the receptor-like cytoplasmic kinase1 and type III effector AvrBsT and promotes the hypersensitive cell death response in a phosphorylation-dependent manner. Plant Physiol. 165, 76-91.
Klebba, P. E. and Newton, S. M. (1998) Mechanisms of solute transport through outer membrane porins: burning down the house. Curr. Opin. Microbiol. 1, 238-247.
Kunz, H.-H., Scharnewski, M., Feussner, K., Feussner, I., FIE ugge, U.-I., Fulda, M. and Gierth, M. (2009) The ABC transporter PXA1 and peroxisomal boxidation are vital for metabolism in mature leaves of arabidopsis during extended darkness. Plant Cell, 21, 2733-2749.
Langley, D. B., Schofield, P., Nevoltris, D., Jackson, J., Jackson, K. J. L., Peters, T. J., Burk, M. et al. (2022) Genetic and structural basis of the human anti-a-galactosyl antibody response. Proc. Natl Acad. Sci. USA, 119, e2123212119.
Lauber, F., Deme, J. C., Lea, S. M. and Berks, B. C. (2018) Type 9 secretion system structures reveal a new protein transport mechanism. Nature, 564, 77-82.
Lee, Y. J. and Jeong, K. J. (2015) Challenges to production of antibodies in bacteria and yeast. J. Biosci. Bioeng. 120, 483-490.
Lee, C.-W., Efetova, M., Engelmann, J. C., Kramell, R., Wasternack, C., Ludwig-Muller, J. et al. (2009) Agrobacterium tumefaciens promotes tumor induction by modulating pathogen defense in Arabidopsis thaliana. Plant Cell, 21, 2948-2962.
Leth, I. K. and McDonald, K. A. (2017) Growth kinetics and scale-up of Agrobacterium tumefaciens. Appl. Microbiol. Biotechnol. 101, 4895-4903.
Liu, S., Jiang, J., Ma, Z., Xiao, M., Yang, L., Tian, B., Yu, Y. et al. (2022) The role of hydroxycinnamic acid amide pathway in plant immunity. Front. Plant Sci. 13, 922119.
van Loon, L. C., Bakker, P. A. H. M., van der Heijdt, W. H. W., Wendehenne, D. and Pugin, A. (2008) Early responses of tobacco suspension cells to rhizobacterial elicitors of induced systemic resistance. Mol. Plant-Microbe Interact. 21, 1609-1621.
Lua, W.-H., Gan, S. K.-E., Lane, D. P. and Verma, C. S. (2015) A search for synergy in the binding kinetics of trastuzumab and pertuzumab whole and F(ab) to Her2. NPJ Breast Cancer, 1, 15012.
Mazumder, M., Das, S., Saha, U., Chatterjee, M., Bannerjee, K. and Basu, D. (2013) Salicylic acid-mediated establishment of the compatibility between Alternaria brassicicola and Brassica juncea is mitigated by abscisic acid in Sinapis alba. Plant Physiol. Biochem. 70, 43-51.
McConn, M., Creelman, R. A., Bell, E., Mullet, J. E. and Browse, J. (1997) Jasmonate is essential for insect defense in Arabidopsis. Proc. Natl Acad. Sci. USA, 94, 5473-5477.
Mclean, M. D. (2017) Trastuzumab made in plants using vivoXPRESS platform technology. J. Drug Des. Res. 4, 1052.
Meissner, F., Geddes-McAlister, J., Mann, M. and Bantscheff, M. (2022) The emerging role of mass spectromerty based proteomics in drug discovery. Nat. Rev. Drug Discov. 21, 637-654.
Mumm, R., Maarten, A. and Dicke, M. (2008) Significance of terpenoids in induced indirect plant defence against herbivorous arthropods. Plant Cell Environ. 31, 575-585.
Myers, E. W., and W. Miller, 1988 Optimal alignments in linear space. Comput Appl Biosci 4:11-17.
Oberpichler, I., Rosen, R., Rasouly, A., Vugman, M., Ron, E. Z. and Lamparter, T. (2008) Light affects motility and infectivity of Agrobacterium tumefaciens. Environ. Microbiol. 10, 2020-2029.
Ogata, H., Goto, S., Sato, K., Fujibuchi, W., Bono, H. and Kanehisa, M. (1999) KEGG: Kyoto Encyclopedia of Genes and Genomes. Nucleic Acids Res. 27, 29-34.
Peart, J. R., Lu, R., Sadanandom, A., Malcuit, I., Moffett, P., Brice, D. C., Schauser, L. et al. (2002) Ubiquitin ligase-associated protein SGT1 is required for host and nonhost disease resistance in plants. Proc. Natl Acad. Sci. USA, 99, 10865-10869.
Pluskal, T., Castillo, S., Villar-Briones, A. and Ore si c, M. (2010) MZmine 2: modular framework for processing, visualizing, and analyzing mass spectrometry-based molecular profile data. BMC Bioinformatics, 11, 395.
Prudhomme, N., Gianetto-Hill, C., Pastora, R., Cheung, W. F., Allen-Vercoe, E., McLean, M. D., Cossar, D. et al. (2021a) Quantitative proteomic profiling of shake flask versus bioreactor growth reveals distinct responses of Agrobacterium tumefaciens for preparation in molecular pharming. Can. J. Microbiol. 67, 75-84.
Prudhomme, N., Pastora, R., Muselius, B., McLean, M. D., Cossar, D. and Geddes-McAlister, J. (2021b) Exposure of Agrobacterium tumefaciens to agroinfiltration medium demonstrates cellular remodeling and may promote enhanced adaptability for molecular pharming. Can. J. Microbiol. 67, 85-97.
Prudhomme, N., Krieger, J. R., McLean, M. D., Cossar, D. and Geddes-McAlister, J. (2022) Proteomic profiling of interplay between Agrobacterium tumefaciens and Nicotiana benthamiana for improved molecular pharming outcomes. Methods Mol. Biol. 2456, 275-286.
Puchol Tarazona, A. A., Maresch, D., Grill, A., Bakalarz, J., Torres Acosta, J. A., Castilho, A., Steinkellner, H. et al. (2021) Identification of two subtilisin-like serine proteases engaged in the degradation of recombinant proteins in Nicotiana benthamiana. FEBS Lett. 595, 379-388.
Rappsilber, J., Mann, M. and Ishihama, Y. (2007) Protocol for micropurification, enrichment, pre-fractionation and storage of peptides for proteomics using StageTips. Nat. Protoc. 2, 1896-1906.
Ruan, J., Zhou, Y., Zhou, M., Yan, J., Khurshid, M., Weng, W., Cheng, J. et al. (2019) Jasmonic acid signaling pathway in plants. Int. J. Mol. Sci. 20, 2479.
Schenk, P. M., Kazan, K., Wilson, I., Anderson, J. P., Richmond, T., Somerville, S. C. and Manners, J. M. (2000) Coordinated plant defense responses in Arabidopsis revealed by microarray analysis. Proc. Natl Acad. Sci. USA, 97, 11655-11660.
Schillberg, S. and Finnern, R. (2021) Plant molecular farming for the production of valuable proteins-critical evaluation of achievements and future challenges. J. Plant Physiol. 258-259, 153359.
Shanmugaraj, B., I Bulaon, C. J. and Phoolcharoen, W. (2020) Plant molecular farming: a viable platform for recombinant biopharmaceutical production. Plants (Basel), 9, 842.
Sharma, E., Anand, G. and Kapoor, R. (2017) Terpenoids in plant and arbuscular mycorrhiza-reinforced defence against herbivorous insects. Ann. Bot. 119, 791-801.
Shinmyo, A. and Kato, K. (2010) Molecular farming: production of drugs and vaccines in higher plants. J. Antibiot. (Tokyo), 63, 431-433.
Shiraku, M. L., Magwanga, R. O., Cai, X., Kirungu, J. N., Xu, Y., Mehari, T. G., Hou, Y. et al. (2021) Functional characterization of GhACX3 gene reveals its significant role in enhancing drought and salt stress tolerance in cotton. Front. Plant Sci. 12, 658755.
Shulaev, V., Silverman, P. and Raskin, I. (1997) Airborne signalling by methyl salicylate in plant pathogen resistance. Nature, 385, 718-721.
Smits, A. H. and Vermeulen, M. (2016) Characterizing protein-protein interactions using mass spectrometry: challenges and opportunities. Trends Biotechnol. 34, 825-834.
Spiegel, H., Boes, A., Perales Morales, C., Rademacher, T. and Buyel, J. F. (2019) Ready-to-use stocks of Agrobacterium tumefaciens can simplify process development for the production of recombinant proteins by transient expression in plants. Biotechnol. J. 14, e1900113.
Stoger, E., Fischer, R., Moloney, M. and Ma, J. K.-C. (2014) Plant molecular pharming for the treatment of chronic and infectious diseases. Annu. Rev. Plant Biol. 65, 743-768.
Stubenrauch, C., Belousoff, M. J., Hay, I. D., Shen, H.-H., Lillington, J., Tuck, K. L., Peters, K. M. et al. (2016) Effective assembly of fimbriae in Escherichia coli depends on the translocation assembly module nanomachine. Nat. Microbiol. 1, 16064.
Sugimoto, K., Matsui, K., Iijima, Y., Akakabe, Y., Muramoto, S., Ozawa, R., Uefune, M. et al. (2014) Intake and transformation to a glycoside of (Z)-3-hexenol from infested neighbors reveals a mode of plant odor reception and defense. Proc. Natl Acad. Sci. USA, 111, 7144-7149.
Suzuki, M., Roy, R., Zheng, H., Woychik, N. and Inouye, M. (2006) Bacterial bioreactors for high yield production of recombinant protein. J. Biol. Chem. 281, 37559-37565.
Tanaka, K. J., Song, S., Mason, K. and Pinkett, H. W. (2018) Selective substrate uptake: the role of ATP-Binding Cassette (ABC) importers in pathogenesis. Biochim. Biophys. Acta Biomembr. 1860, 868-877.
Tschofen, M., Knopp, D., Hood, E. and St€oger, E. (2016) Plant molecular farming: much more than medicines. Annu Rev Anal Chem (Palo Alto, Calif), 9, 271-294.
Tsugawa, H., Cajka, T., Kind, T., Ma, Y., Higgins, B., Ikeda, K., Kanazawa, M. et al. (2015) MS-DIAL: data-independent MS/MS deconvolution for comprehensive metabolome analysis. Nat. Methods, 12, 523-526.
Tyanova, S., Temu, T., Sinitcyn, P., Carlson, A., Hein, M. Y., Geiger, T., Mann, M. et al. (2016) The Perseus computational platform for comprehensive analysis of (prote) omics data. Nat. Methods, 13, 731-740.
Valanciene, E. and Malys, N. (2022) Advances in production of hydroxycinnamoyl-quinic acids: from natural sources to biotechnology. Antioxidants, 11, 2427.
Veena, J. H., Doerge, R. W. and Gelvin, S. B. (2003) Transfer of T-DNA and Vir proteins to plant cells by Agrobacterium tumefaciens induces expression of host genes involved in mediating transformation and suppresses host defense gene expression. Plant J. 35, 219-236
Wang, Y., Li, X., Chen, X., Nielsen, J., Petranovic, D. and Siewers, V. (2021) Expression of antibody fragments in Saccharomyces cerevisiae strains evolved for enhanced protein secretion. Microb. Cell Fact. 20, 134.
Xu, X. Q. and Pan, S. Q. (2000) An Agrobacterium catalase is a virulence factor involved in tumorigenesis. Mol. Microbiol. 35, 407-414.
Yan, J., Risacher, S. L., Shen, L. and Saykin, A. J. (2017) Network approaches to systems biology analysis of complex disease: integrative methods for multiomics data. Brief. Bioinform. 19, 1370-1381.
Yee, C. M., Zak, A. J., Hill, B. D. and Wen, F. (2018) The coming age of insect cells for manufacturing and development of protein therapeutics. Ind. Eng. Chem. Res. 57, 10061-10070.
Yu, S.-H., Kyriakidou, P. and Cox, J. (2020) Isobaric matching between runs and novel PSM-level normalization in MaxQuant strongly improve reporter ionbased quantification. J. Proteome Res. 19, 3945-3954.
Zdyb, A., SaIgado, M. G., Demchenko, K. N., Brenner, W. G., P·aszczyca, M., Stumpe, M., Herrfurth, C. et al. (2018) Allene oxide synthase, allene oxide cyclase and jasmonic acid levels in Lotus japonicus nodules. PLOS One, 13, e0190884.
Zhang, W., Zhao, F., Jiang, L., Chen, C., Wu, L. and Liu, Z. (2018) Different pathogen defense strategies in Arabidopsis: more than pathogen recognition. Cells, 7, 252.
Zhu, M. M., Mollet, M., Hubert, R. S., Kyung, Y. S. and Zhang, G. G. (2017) Industrial production of therapeutic proteins: cell lines, cell culture, and purification. In Handbook of Industrial Chemistry and Biotechnology (Kent, J., Bommaraju, T. and Bamicki, S., eds), pp. 1639-1669. Cham: Springer International Publishing.

1. A method of enhancing production of a heterologous protein in a plant or plant cell comprising:
a) introducing into the plant or plant cell a nucleic acid molecule encoding the heterologous protein operably linked to a promoter;
b) inhibiting aspartic protease AED3-1; and
c) growing the plant or plant cell to obtain a plant that expresses the heterologous protein.

2. The method of claim 1, wherein step a) further comprises introducing into the plant or plant cell a nucleic acid molecule encoding a suppressor of gene silencing protein operably linked to a promoter, optionally P19.

3. The method of claim 1, wherein the plant or plant cell is of genus Nicotiana, optionally Nicotiana benthamiana (N. benthamiana).

4. The method of claim 1, wherein the AED3-1 comprises i) an amino acid sequence according to SEQ ID NO: 1 or ii) an amino acid sequence with at least 60% sequence identity to SEQ ID NO: 1.

5. The method of claim 1, wherein the inhibiting in b) comprises contacting the plant or plant cell with an inhibitor of AED3-1.

6. The method of claim 5, wherein the inhibitor comprises:
a polypeptide;
a small molecule drug;
a short hairpin RNA (shRNA), a small interfering RNA (siRNA), a micro RNA (miRNA), or an antisense oligonucleotide (ASO); and/or
a nucleotide comprising a nucleic acid sequence according to SEQ ID NO: 2 or a functional variant thereof.

7. The method of claim 1, wherein the inhibiting in b) comprises:
introducing into the plant or plant cell a nucleic acid molecule encoding the inhibitor of AED3-1 operably linked to a promoter; and/or
introducing into the plant or plant cell at least one genomic modification in a gene encoding the AED3-1, wherein the at least one genomic modification confers reduced or loss of function of the AED3-1 as compared to a plant of the same species lacking the at least one genomic modification.

8. The method of claim 1, wherein the heterologous protein is:
a biologic drug;
an antibody, optionally a human antibody or a humanized antibody;
trastuzumab;
insulin;
a growth hormone; or
a vaccine.

9. The method of claim 1, wherein the introducing into the plant comprises agroinfiltration, optionally with Agrobacterium tumefaciens (A. tumefaciens).

10. The method of claim 9, wherein the A. tumefaciens is grown in a bioreactor prior to agroinfiltration.

11. The method of claim 9, wherein the heterologous protein is harvested less than about 7 days post-infiltration (dpi) or at about 3 dpi.

12. A modified plant or plant cell with enhanced production of a heterologous protein, comprising:
a) a nucleic acid molecule encoding the heterologous protein operably linked to a promoter; and
b) at least one of the group consisting of:
i) an inhibitor of aspartic protease AED3-1; and
ii) at least one genomic modification in a gene encoding aspartic protease AED3-1 conferring reduced or loss of function of the AED3-1 as compared to a plant of the same species lacking the at least one genomic modification.

13. The modified plant or plant cell of claim 12, further comprising a nucleic acid molecule encoding a suppressor of gene silencing protein operably linked to a promoter, optionally P19.

14. The modified plant or plant cell of claim 12, wherein the plant or plant cell is of genus Nicotiana, optionally Nicotiana benthamiana (N. benthamiana).

15. The modified plant or plant cell of claim 12, wherein the AED3-1 comprises i) an amino acid sequence according to SEQ ID NO: 1 or ii) an amino acid sequence with at least about 60% sequence identity to SEQ ID NO: 1.

16. The modified plant or plant cell of claim 12, wherein the inhibitor of AED3-1 comprises:
a polypeptide;
a small molecule drug;
a short hairpin RNA (shRNA), a small interfering RNA (siRNA), a micro RNA (miRNA), or an antisense oligonucleotide (ASO); and/or
a nucleotide comprising a nucleic acid sequence according to SEQ ID NO: 2 or a functional variant thereof.

17. The modified plant or plant cell of claim 12, wherein the at least one genomic modification conferring reduced or loss of function comprises an insertion, a deletion, or a substitution.

18. The modified plant or plant cell of claim 12, wherein the heterologous protein is:
a biologic drug;
an antibody, optionally a human antibody or humanized antibody;
trastuzumab;
insulin;
a growth hormone; or
a vaccine.

19. A nucleic acid molecule, comprising (a) a nucleic acid sequence according to SEQ ID NO: 2 or a reverse complement thereof, or (b) a functional variant of (a).

20. The nucleic acid molecule of claim 19, wherein the nucleic acid molecule is a short hairpin RNA (shRNA).


SEQUENCE LISTING (6 sequences)

SEQ ID NO: 1 - protein, 438 aa, Nicotiana tabacum
MEGSIIFLLSLLLFFTLAKGLTNPKCGSSFPQADKGSTLQVLHVNSPCSPLRNNAPQSWVDTVLQMQSKDQARLDLFASLVAGRSFVPIASGRQVIQSPTYIVRAKIGTPPQTLLVAVDNSNDVAWFPCSGCVGCSSTVFASDKSTTFKNVSCGAAQCSQVPNPTCGGSSCGFNLTYGGSSIAANLSQDTLTLATDAVPSYTFGCVQKATGSSAPPQGLLGLGRGPLSFLSQTQSHYQSTFSYCLPSYKSPNFSGTLRLGPNGQPKRIKTTQLLRNPRRSSFYYVNLVGVKVGRRIVDIPPSALAFNPSTGAGTIIDSGTVFTRLVEPAYTAVRNEFRRRMGRNTTVTSLGGFDTCYTVPITIPTITLMFAGMNVTLPQDNFLIRSSSSSTTCLAMAASPADPVNSVLNVIANWQQQNHRFLFDVPNSKLGVARETCS

SEQ ID NO: 2 - RNA, 59 nt, synthetic construct
  source 1..59: mol_type = other RNA
ccggtgttggttgctgtggataatatcaagagtattatccacagcaaccaacatttttg

SEQ ID NO: 3 - DNA, 79 bp, synthetic construct
  source 1..79: mol_type = other DNA
aattaagcttccggtgttggttgctgtggataatatcaagagtattatccacagcaaccaacatttttggagctcatat

SEQ ID NO: 4 - DNA, 79 bp, synthetic construct
  source 1..79: mol_type = other DNA
atatgagctccaaaaatgttggttgctgtggataatactcttgatattatccacagcaaccaacaccggaagcttaatt

SEQ ID NO: 5 - DNA, 2681 bp, Nicotiana tabacum
  source 1..2681: mol_type = genomic DNA
taaaccacaccaaaagccaaagtactctcacaagccaatggagggctctatcattttcttactatcccttctcttatttttcactctagccaaagggctaaccaaccccaaatgtggcagcagctttccccaagcagacaaaggctcaacactacaagtcctacatgtgaacagcccttgctctcctcttaggaacaatgctccccagtcatgggttgatactgtcctccaaatgcagtccaaagaccaagccagacttgatttgtttgctagtcttgtggctgggagatcttttgttccaattgcttcgggaagacaagttatacagagcccaacttacatagtgagggccaagattggaaccccacctcaaaccttgttggttgctgtggataatagcaatgatgttgcttggttcccttgcagtggttgtgttggttgctcctctactgtttttgcatcagacaagtccaccactttcaagaatgttagctgtggagctgcacaatgcagccaggtaccccattatacctttcaaaccaactttattatacacctttcttacattttactctctccgttttgttttataaaaatgccatttttgtctcgacataaaatttaagaagtaaaaaaaggcttttgacatgtattaaagtgtccttgtaacttgtaaccatatcactacatctctttagctataagagttgtataggaagtttaatgtaaaagagttcatgaaggtgttattcttgttaaaataaattataatggaaagagtgtcagtacaaaatggaatggaggaacagcattttgaaagatttcctaatttcctaaactagtttttcgtgcatttaacttctaatgttgaagtactagaaattatctacatttctgcagcgatagcatatcaaccacatgttttttatatactactagtagtatataagagcacacctaccacatgtaacgctttctctgtcttcaatttaatgatcctattcggctcaaaataggcttagagcacttcgtataaataggattaaacagctttcaacataataatctattaagcacaactttcaggataagaaaatgatcataatttaagaaaagctagtgaattttttaaaataggaaacgagcttcgtatggcgcgtgggaactgaaaagttgtttcctctttcctggctttgtcgacattggagcaattagcggtagagttatccgagataactaatcccgtgacccttagtcacatgatttggtcaacattctcattccacaaaagaacatcatttatttgtaagacacttttatgtcgctaaaatataagatgttactggtaaaatggacaataggaattccaagtataaagcacaataactaggtttcagtatcaaataagttcagctggactatatgaaattttacacaacaagatttatttgtgtatatgctaattattactaaaaaaatatttttctcaggtaccaaatcccacatgcggtggcagcagctgcggcttcaacctaacctacggcggctccagcatagctgcaaatctctcacaagacacattgacactcgccactgacgccgtgccttcatatacctttggttgtgtacaaaaggccaccggcagctccgcgccaccccaggggctattaggtttgggtcgaggcccattgtcatttttgtcccaaacccaaagccattaccagtctacattctcctactgtttgcccagttacaagtctcccaacttttccggcacactcaggttgggcccaaatggccagcccaaaaggattaagacaactcaattgctaagaaacccaaggagatcttccttttattatgtcaacttggtcggagttaaagtcggccggagaatcgttgacatccctcccagtgctttggctttcaacccttccaccggtgccggcaccataattgattcgggtatttaatttaacaatgatcaatatatctgtaattataattctatgtaccaatttttgctaaaaggcattgtcaatcgacaggaacggtattcacgaggctagtggagccagcgtatacggcagtgaggaacgagtttaggaggagaatgggaaggaacacgaccgtgacaagccttggcggattcgacacttgctacaccgtccccattacaattccaacaataacgctgatgtttgcgggcatgaacgtgacgctgccgcaagacaacttcttaatccgcagcagttccagcagtacaacttgcctcgctatggctgcttccccagccgaccctgtcaattccgtcctcaacgtcatcgccaactggcagcaacagaatcaccgcttcctctttgacgttcctaattctaagctcggcgttgctcgtgaaacctgcagctgaagtactgcttttggattttaatgggattttctccttttggctctttccgttttttcttttttcttaacgtggggaattaattaattattagagatagttttcacggtttggcactttgatattgtcggtgcattttgctgttttggatttatcttatctgtgtcacgggttgaaagttactacacctgtttccaactattatcctcctatttatttcttggtttccccaccttgaaggaaaacagaaaatgattgctcgtccattccagtt

SEQ ID NO: 6 - DNA, 519 bp, synthetic construct
  source 1..519: mol_type = other DNA
atggaaagggctattcagggaaatgatgctagagagcaggctaattctgaaagatgggatggtggatctggtggaactacttctccattcaagcttccagatgagtctccatcttggactgagtggaggcttcataacgatgagactaactccaatcaggataacccactcggattcaaagaatcttggggattcggaaaggttgtgttcaagcgttaccttaggtatgataggactgaggcttcacttcatagggttctcggatcttggactggtgattctgttaactacgctgcttctcgtttttttggattcgatcagatcggatgcacttactctattaggttcaggggagtgtctattactgtttctggtggatctaggactcttcaacacctttgcgagatggctattaggtctaagcaagagcttcttcagcttgctccaattgaggttgagtctaacgtttcaagaggatgtccagaaggtactgagactttcgagaaagaatccgagtga